(19) United States
(12) Patent Application Publication
(10) Pub. No.: US 2004/0186154 A1
(43) Pub. Date: Sep. 23, 2004

(54) Therapeutic combinations for cardiovascular and inflammatory indications

(76) Inventors: Karen Seibert, St Louis, MO (US); Bradley T Keller, Chesterfield, MO (US); Peter C Isakson, Morristown, NJ (US)

Correspondence Address: BANNER & WITCOFF, 1001 G STREET N W, SUITE 1100, WASHINGTON, DC, 20001, US

(21) Appl. No.: 10/473,045

(22) PCT Filed: Mar. 28, 2002
(86) PCT No.: PCT/US02/09346

Related U.S. Application Data
(60) Provisional application No. 60/279,239, filed on Mar. 28, 2001.

(51) Int. Cl. A61K 31/415; A61K 31/365

(52) U.S. Cl. 514/406; 514/471

(57) ABSTRACT

The present invention provides therapeutic combinations and methods for treating or preventing a hypercholesterolemia-related or an inflammation-related condition in a subject in need of such treatment or prevention. One therapeutic combination comprises an ASBT inhibitor combined with COX-2 inhibitor. A further therapeutic combination comprises an ASBT inhibitor, a COX-2 inhibitor and an HMG Co-A reductase inhibitor. Another therapeutic combination comprises a chromene COX-2 inhibitor and an HMG Co-A reductase inhibitor.

This application claims priority to U.S. Provisional Application No. 60/279,239 ('239) filed on Mar. 28, 2001 before the United States Patent & Trademark Office. The above-noted '239 U.S. Provisional Application is incorporated herein by reference in its entirety for all purposes.

### BACKGROUND OF THE INVENTION

1. Field of the Invention

The present invention relates to methods of treating cardiovascular, inflammatory and other diseases, and specifically relates to combinations of compounds, compositions, and methods for their use in medicine, particularly in the prophylaxis and treatment of hyperlipidemic or inflammatory conditions such as are associated with atherosclerosis, hypercholesterolemia, coronary plaque inflammation and other cardiovascular diseases in mammals. More particularly, the invention relates to apical sodium co-dependent bile acid transport inhibitors, cyclooxygenase inhibitors (e.g., cyclooxygenase-2 selective inhibitors), and HMG-CoA reductase inhibitors.

2. Description of Related Art

It is well-settled in the literature that hyperlipidemic conditions associated with elevated concentrations of total cholesterol and low-density lipoprotein (LDL) cholesterol are major risk factors for coronary heart disease and particularly atherosclerosis. More recently, the role of inflammation in cardiovascular diseases has become much better understood. These findings serve to point out the acute need for prophylactic and therapeutic strategies for cardiovascular disease that are effective in simultaneously controlling both inflammatory and hyperlipidemic conditions.

The non-steroidal anti-inflammatory drugs (NSAIDs) are known to prevent the formation of prostaglandins by inhibiting enzymes in the human arachidonic acid/prostaglandin pathway, in particular the enzyme cyclooxygenase (COX). For this reason the NSAIDs are effective in reducing the prostaglandin-induced pain and swelling associated with inflammatory processes. The recent discovery that there are two isoforms of the COX enzyme, COX-1 and COX-2, has given rise to new approaches for NSAID discovery and utilization, because it has been shown that COX-2 is the isoform specifically induced in many inflamed tissues. Many compounds have been identified which have activity as COX-2 inhibitors. A recent review of COX-2 selective inhibitors is provided by Carty and Marfat (Current Opinion in Anti-inflammatory & Immunomodulatory Investigational Drugs, 1 (20), 89-96 (1999)).

Atherosclerosis underlies most manifestations of coronary artery disease (CAD), a major cause of morbidity and mortality in modern society. High LDL cholesterol (above about 180 mg/dl) and low HDL cholesterol (below 35 mg/dl) have been shown to be important contributors to the development of atherosclerosis. Other diseases or risk factors, such as peripheral vascular disease, stroke, and hypercholesterolemia are also negatively affected by adverse HDL/LDL ratios.

A metabolic equilibrium generally exists between hepatic cholesterol and the bile acid pool. Interruption of the enterohepatic recirculation of bile acids results in a decrease in the liver bile acid pool and stimulates increased hepatic synthesis of bile acids from cholesterol, eventually depleting the liver's pool of esterified cholesterol. In order to maintain the liver cholesterol levels necessary to support bile acid synthesis, de novo synthesis of cholesterol increases in hepatocytes via an up-regulation of the activity of 3-hydroxy-3-methylglutaryl coenzyme-A reductase (HKG-CoA reductase), while liver uptake of serum cholesterol is increased as a result of the up-regulation of the number of hepatic cell surface receptors for low density lipoprotein cholesterol. The latter increase in hepatic receptors directly leads to a reduction in serum LDL cholesterol levels. Abundant epidemiological data have accumulated which indicate that such reduction leads to significant mitigation of the disease symptoms of atherosclerosis. The discovery of specific ASBT inhibitors is further reviewed by Booker and Arbeeny (Cardiovasc. Pulmon. Renal Invest. Drugs, 2, 208-215(2000)).

Various benzothiepine inhibitors of bile acid absorption have been disclosed by G. D. Searle (PCT Pat. Appl. WO 93/321146) for numerous uses, including regulation of fatty acid metabolism and treatment of coronary vascular disease.

PCT patent application No. WO 92/18462 lists other benzothiepines for use as hypolipemic and hypocholesterolemic agents. Each of the benzothiepine hypolipemic and hypocholesterolemic agents described in these individual patent applications is limited by an amide bonded to the carbon adjacent the phenyl ring of the fused bicyclobenzothiepine ring.

PCT patent application no. WO 93/16055, which describes a number of hypolipidemic benzothiazepine compounds. Additional hypolipidemic benzothiazepine compounds (particularly 2,3,4,5-tetrahydrobenzo-1-thi-4-azepine compounds) are disclosed in another PCT patent application no. WO 96/05188. Further hypolipidemic benzothiazepine compounds are also described in another world patent application (28).

Further ASBT inhibitor compounds include a class of lignan derivatives as described by Takashima et al. (Atherosclerosis, 107, 247-257 (1994)).

Another approach to the reduction of total cholesterol relies on the understanding that HMG-CoA reductase catalyzes the rate-limiting step in the biosynthesis of cholesterol (The Pharmacological Basis of Therapeutics, 9th ed., J. G. Hardman and L. E. Limberd, ed., McGraw-Hill, Inc., New York, pp. 884-888 (1996)). HMG-CoA reductase inhibitors (including the class of therapeutics commonly called “statins”) reduce blood serum levels of LDL cholesterol by competitive inhibition of this biosynthetic step.

Numerous antihyperlipidemic agents having other modes of action also have been disclosed in the literature as being useful for the treatment of hyperlipidemic conditions and disorders. These agents include, for example, commercially available drugs such as nicotinic acid, bile acid sequestrants including cholestryramine and colestipol, probucol, and fibric acid derivatives including gemfibrozil and clofibrate.

Some combination therapies for the treatment of cardiovascular disease have been described in the literature. A combinations of an ASBT inhibitor with HMG-a CoA reductase inhibitor useful for the treatment of cardiovascular disease is disclosed in PCT patent application no. WO 98/40375.

PCT Patent Application No. WO 99/20110 describes a therapeutic combination of a COX-2 selective inhibitor with an HMG CO-A reductase inhibitor.

While the above references indicate the value of the known combination therapies in reducing the impact of hyperlipidemia on cardiovascular disease, there is a continuing urgent need to find safe, effective agents for the prophylaxis or treatment of cardiovascular and metabolic diseases involving both inflammatory and hyperlipidemic conditions. The novel combinations of the present invention exhibit improved efficacy, improved potency, and/or reduced dosing requirements for the active compounds relative to combination regimens previously disclosed in the published literature.

### SUMMARY OF THE INVENTION

To address the continuing need to find safe and effective agents for the prophylaxis and treatment of cardiovascular and other diseases, combination therapies of anti-inflammatory and anti-hyperlipidemic drugs are now disclosed.

Among its several embodiments, the present invention provides a combination therapy comprising treating a subject with an amount of an apical sodium co-dependent bile acid transport inhibitor and an amount of a cyclooxygenase-2 (COX-2) selective inhibitor or its prodrug, wherein the amount of the apical sodium co-dependent bile acid transport (ASBT) inhibitor and the amount of the cyclooxygenase-2 (COX-2) selective inhibitor together constitute a hypercholesterolemia-related condition effective amount or an inflammation-related condition effective amount of the compounds. For example, one of the many embodiments of the present invention is a combination therapy comprising therapeutic dosages of an ASBT inhibitor selected from Table 2 and a cyclooxygenase-2 (cox-2) selective inhibitor selected from Tables 4, 6 and 7A. A preferred embodiment of the present invention is a combination therapy comprising therapeutic dosages of a bicyclic benzothiepine ASBT inhibitor and a tricyclic cyclooxygenase-2 selective inhibitor.

In another embodiment, the present invention comprises a therapeutic combination containing an amount of an apical sodium co-dependent bile acid transport (ASBT) inhibitor and an amount of a cyclooxygenase-2 (COX-2) selective inhibitor or its prodrug, and a pharmaceutically acceptable carrier, wherein the amount of the apical sodium co-dependent bile acid transport (ASBT) inhibitor and the amount of the cyclooxygenase-2 (COX-2) selective inhibitor together constitute a hypercholesterolemia-related condition effective amount or an inflammation-related condition effective amount of the said compounds. For example, one of the many embodiments of the present invention is a combination comprising therapeutic dosages of an ASBT inhibitor selected from Table 2 and a cyclooxygenase-2 selective inhibitor selected from Tables 4, 6 and 7A. A preferred embodiment of the present invention is a combination comprising therapeutic dosages of a benzothiepine ASBT inhibitor and a tricyclic cyclooxygenase-2 selective inhibitor.

Alternatively, an aspect of the present invention is a cardiovascular combination therapy comprising treating a subject with an amount of an apical sodium co-dependent bile acid transport inhibitor and an amount of a cyclooxygenase-2 (COX-2) selective inhibitor or its prodrug and an amount of an HMG-CoA reductase inhibitor, wherein the amount of the apical sodium co-dependent bile acid transport inhibitor, the amount of the cyclooxygenase-2 (COX-2) selective inhibitor and the amount of the HMG-CoA reductase inhibitor together constitute a hypercholesterolemia-related condition effective amount or an inflammation-related condition effective amount of the said compounds. For example, one of the many embodiments of the present invention is a combination therapy comprising therapeutic dosages of an ASBT inhibitor selected from Table 2 and a cyclooxygenase-2 selective inhibitor selected from Tables 4, 6 and 7A and an HMG-CoA inhibitor selected from Table 8. A preferred embodiment of the present invention is a combination therapy comprising therapeutic dosages of a benzothiepine ASBT inhibitor, a tricyclic cyclooxygenase-2 (COX-2) selective inhibitor and a statin HMG-CoA inhibitor.

In yet another embodiment, the present invention comprises a therapeutic combination containing an amount of an apical sodium co-dependent bile acid transport inhibitor, an amount of a cyclooxygenase-2 (COX-2) selective inhibitor or its prodrug and an amount of an HMG-CoA reductase inhibitor, and a pharmaceutically acceptable carrier, wherein the amount of the apical sodium co-dependent bile acid transport inhibitor, the amount of the cyclooxygenase-2 (COX-2) selective inhibitor and the amount of the HMG-CoA inhibitor together constitute a hypercholesterolemia-related condition effective amount or an inflammation-related condition effective amount of the said compounds. For example, one of the many embodiments of the present invention is a combination comprising therapeutic dosages of an ASBT inhibitor selected from Table 2 and a cyclooxygenase-2 (COX-2) selective inhibitor selected from Tables 4, 6 and 7A and an HMG-CoA inhibitor selected from Table 8. A preferred embodiment of the present invention is a combination comprising therapeutic dosages of a benzothiepine ASBT inhibitor, a tricyclic cyclooxygenase-2 selective inhibitor and a statin HMG-CoA inhibitor.

In a further embodiment, the present invention provides a method for treating or preventing a hypercholesterolemia-related or an inflammation-related condition in a subject in need of such treatment or prevention, comprising treating the subject with an amount of an apical sodium co-dependent bile acid transport (ASBT) inhibitor and an amount of a chromene cyclooxygenase inhibitor (e.g., chromene cyclooxygenase-2 (COX-2) selective inhibitor) or its prodrug, wherein the amount of the apical sodium co-dependent bile acid transport inhibitor and the amount of the chromene cyclooxygenase inhibitor (e.g., chromene cyclooxygenase-2 (COX-2) selective inhibitor) together constitute a hypercholesterolemia-related condition effective amount or an inflammation-related condition effective amount of the apical sodium co-dependent bile acid transport inhibitor and the chromene cyclooxygenase inhibitor (e.g., chromene cyclooxygenase-2 (COX-2) selective inhibitor).

In a further embodiment, the present invention provides a method for treating or preventing a hypercholesterolemia-related or an inflammation-related condition in a subject in need of such treatment or prevention, comprising treating the subject with an amount of an HMG Co-A reductase inhibitor and an amount of a chromene cyclooxygenase inhibitor (e.g., chromene cyclooxygenase-2 (COX-2) selective inhibitor) or its prodrug, wherein the amount of the HMG Co-A reductase inhibitor and the amount of the chromene cyclooxygenase inhibitor (e.g., chromene cyclooxygenase-2 (COX-2) selective inhibitor) together constitute a hypercholesterolemia-related condition effective amount or an inflammation-related condition effective amount of the HMG Co-A reductase inhibitor and the chromene cyclooxygenase inhibitor (e.g., chromene cyclooxygenase-2 (COX-2) selective inhibitor).

The present invention also provides a method for treating or preventing a hypercholesterolemia-related or an inflammation-related condition in a subject in need of such treatment or prevention, comprising treating the subject with an amount of an HMG Co-A reductase inhibitor and an amount of a source of valdecoxib, wherein the amount of the HMG Co-A reductase inhibitor and the amount of the source of valdecoxib together constitute a hypercholesterolemia-related condition effective amount or an inflammation-related condition effective amount of the HMG Co-A reductase inhibitor and the source of valdecoxib.

Further scope of the applicability of the present invention will become apparent from the detailed description provided below. However, it should be understood that the following detailed description and examples, while indicating preferred embodiments of the invention, are given by way of illustration only, since various changes and modifications within the spirit and scope of the invention will become apparent from this detailed description to those skilled in the art.

### DETAILED DESCRIPTION OF THE PREFERRED EMBODIMENTS

The following detailed description is provided to aid those skilled in the art in practicing the present invention. Even so, this detailed description should not be construed to unduly limit the present invention, inasmuch as modifications and variations in the embodiments discussed herein can be made by those of ordinary skill in the art without departing from the spirit or scope of the present inventive discovery.

The contents of each of the references cited herein, including the contents of the references cited within these primary references, are herein incorporated by reference in their entirety for all purposes.

a. Definitions

The following definitions are provided in order to aid the reader in understanding the detailed description of the present invention:

The term “subject” as used herein refers to an animal, preferably a mammal, and particularly a human being, who has been the object of treatment, observation or experiment.

The terms “dosing” and “treatment” refer to any process, action, application, therapy, or the like, wherein a subject, and particularly a human being, is rendered medical aid with the object of improving the subject's condition, either directly or indirectly.

“Therapeutic compound” means a compound useful in the prophylaxis or treatment of a hyperlipidemic and/or inflammatory condition, including atherosclerosis, plaque inflammation and hypercholesterolemia.

“Combination therapy” means the administration of two or more therapeutic compounds to treat a hyperlipidemic and/or inflammatory condition, for example atherosclerosis, plaque inflammation, and hypercholesterolemia. Such administration encompasses co-administration of these therapeutic compounds in a substantially simultaneous manner, such as in a single capsule having a fixed ratio of active ingredients or in multiple, separate capsules for each compound. In addition, such administration also encompasses use of each type of therapeutic compound in a sequential manner. In either case, the treatment regimen will provide beneficial effects of the drug combination in treating the cardiovascular or other condition.

The term “therapeutic combination” refers to the administered therapeutic compounds themselves and to any pharmaceutically acceptable carriers used to provide dosage forms such that the beneficial effect of each therapeutic compound is realized by the subject at the desired time, whether the compounds are administered substantially simultaneously or sequentially.

The phrase “therapeutically effective” is intended to qualify the combined amount of therapeutic compounds in the combination therapy. This combined amount will achieve the goal of avoiding or reducing or eliminating the hyperlipidemic condition and/or inflammatory condition.

The terms “cyclooxygenase-2 selective inhibitor” and “COX-2 selective inhibitor” interchangeably refer to a therapeutic compound which preferentially inhibits the COX-2 isoform of the enzyme cyclooxygenase.

The terms “cyclooxygenase-2 nonselective inhibitor” and “COX-2 nonselective inhibitor” interchangeably refer to a therapeutic compound which comparably inhibits both the COX-1 and COX-2 isoforms of the enzyme cyclooxygenase.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598, herein incorporated by reference.

b. Combinations

The combinations of the present invention will have a number of uses. For example, through dosage adjustment and medical monitoring, the individual dosages of the therapeutic compounds used in the combinations of the present invention will be lower than are typical for dosages of the therapeutic compounds when used in monotherapy. The dosage lowering will provide advantages including reduction of side effects of the individual therapeutic compounds when compared to monotherapy. In addition, fewer side effects of the combination therapy compared with monotherapies will lead to greater patient compliance with therapy regimens.

Another use of the present invention will be in combinations having complementary effects or complementary modes of action. For example, ASBT inhibitors frequently lower LDL lipoprotein but also induce de novo synthesis of cholesterol via upregulation of 3-hydroxy-3-methylglutaryl coenzyme-A reductase (HMG-CoA reductase) activity. In contrast, HMG-CoA reductase inhibitors curtail the biosynthesis of cholesterol via inhibition of HMG-CoA reductase. A therapeutic combination of an ASBT inhibitor and a HMG-CoA reductase inhibitor will, when dosages are optimally adjusted, significantly lower LDL and reduce the biosynthesis of new cholesterol.

c. ASBT Inhibitors

The present invention discloses that treatment of a subject with one or more ASBT inhibitors and one or more cyclooxygenase-2 selective inhibitors results in the prophylaxis and/or treatment of cardiovascular conditions and/or disorders relative to other combination regimens. The method comprises treating the subject with an amount of an apical sodium co-dependent bile acid transport inhibitor and an amount of a cyclooxygenase-2 selective inhibitor or its prodrug, wherein the amount of the apical sodium co-dependent bile acid transport inhibitor and the amount of the cyclooxygenase-2 selective inhibitor together constitute a hypercholesterolemia-related condition effective amount or an inflammation-related condition effective amount of the said compounds.

For example, one of the many embodiments of the present invention is a combination therapy comprising therapeutic dosages of a cyclooxygenase-2 selective inhibitor and a lignan ASBT inhibitor selected from the group of lignan ASBT inhibitors illustrated in Table 2 as compounds A-2 and A-3.

In another embodiment of the invention the ASBT inhibitor is selected from the group of bicyclic benzothiazepine ASBT inhibitors illustrated in Table 2 as compounds A-1, A-4 and A-5, including the diastereomers, enantiomers, racemates, salts, tautomers, conjugate acids, and prodrugs thereof.

In a preferred embodiment of the invention the ASBT inhibitor is selected from the group of benzothiepine ASBT inhibitors having the general Formula I shown below and possessing, by way of example and not limitation, the structures A-6 through A-22 disclosed in Table 2, including the diastereomers, enantiomers, racemates, salts, tautomers, conjugate acids, and prodrugs thereof.

TABLE 2

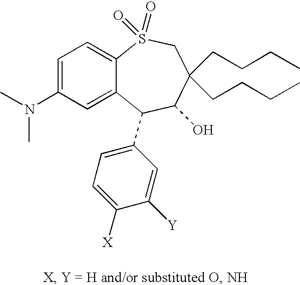

Examples of ASBT Inhibitors as Embodiments

| Compound Number | Structural Formula |
|---|---|
| A-1 | 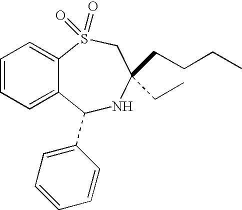 |
| A-2 | 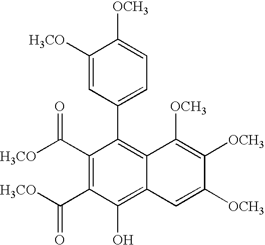 |
| A-3 | 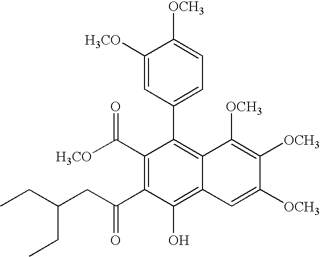 |
| A-4 | 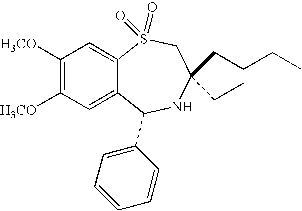 |
| A-5 | 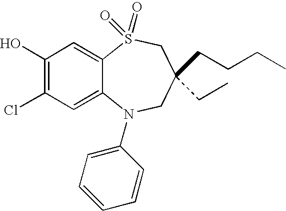 |
| A-6 | 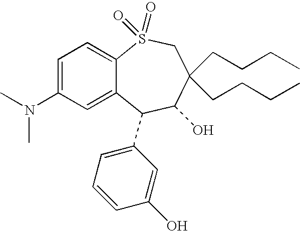 |
| A-7 | 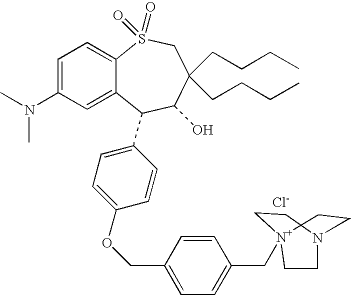 |
| A-8 | 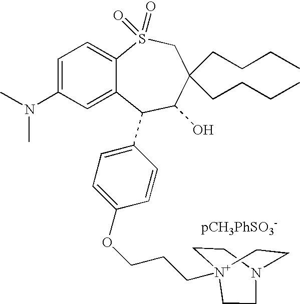 |
| A-9 | 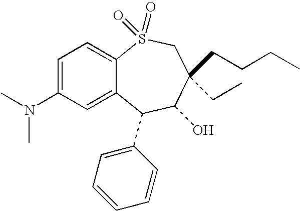 |
| A-10 | 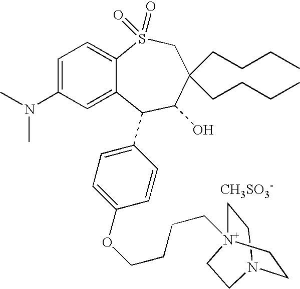 |
| A-11 | 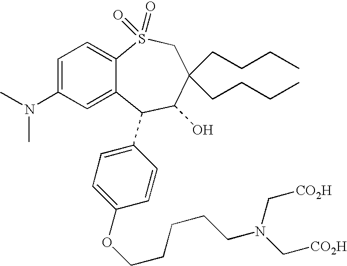 |
| A-12 | 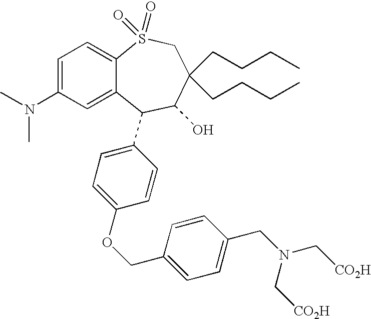 |
| A-13 | 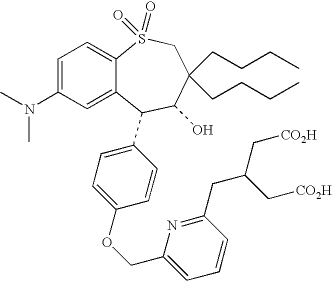 |
| A-14 | 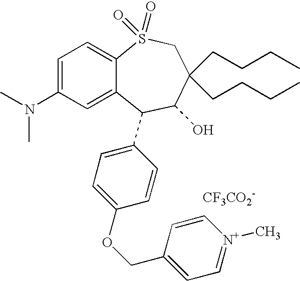 |
| A-15 | 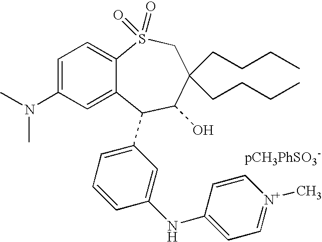 |
| A-16 | 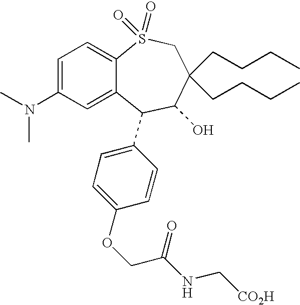 |
| A-17 | 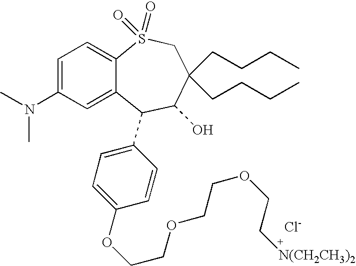 |
| A-18 | 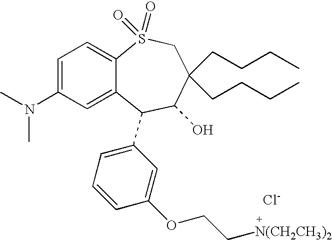 |
| A-19 | 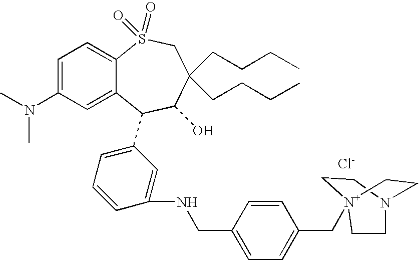 |
| A-20 | 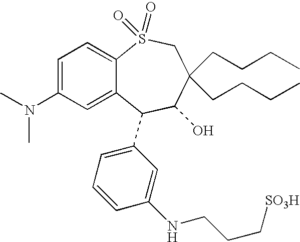 |
| A-21 | 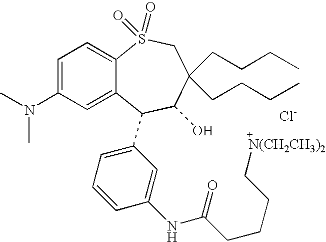 |
| A-22 | 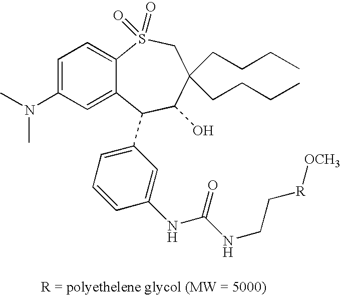 |

The individual patent documents referenced in Table 3 below describe the preparation of the aforementioned ASBT inhibitors of Table 2 and are each herein incorporated by reference.

TABLE 3
References for Preparation of ASBT Inhibitors

| | Patent/Literature Reference for |
|---|---|
| Compound Number | Preparation of Compound Per Se |
| A-1 | U.S. Pat. No. 5,817,652 |
| A-2 | Atherosclerosis, 107, 247-257 |
| | (1994) |
| A-3 | WO 94/24087 |
| A-4 | U.S. Pat. No. 5,910,494 |
| A-5 | WO 99/35135 |
| A-6 | U.S. Pat. No. 5,994,391 |
| A-7 | U.S. Pat. No. 5,994,391 |
| A-8 | U.S. Pat. No. 5,994,391 |
| A-9 | U.S. Pat. No. 5,994,391 |
| A-10 | U.S. Pat. No. 5,994,391 |
| A-11 | U.S. Pat. No. 5,994,391 |
| A-12 | U.S. Pat. No. 5,994,391 |
| A-13 | U.S. Pat. No. 5,994,391 |
| A-14 | U.S. Pat. No. 5,994,391 |
| A-15 | U.S. Pat. No. 5,994,391 |
| A-16 | U.S. Pat. No. 5,994,391 |
| A-17 | U.S. Pat. No. 5,994,391 |
| A-18 | U.S. Pat. No. 5,994,391 |
| A-19 | U.S. Pat. No. 5,994,391 |
| A-20 | U.S. Pat. No. 5,994,391 |
| A-21 | U.S. Pat. No. 5,994,391 |
| A-22 | U.S. Pat. No. 5,994,391 |

Another embodiment of the present invention comprises a pharmaceutical combination containing an amount of an apical sodium co-dependent bile acid transport inhibitor and an amount of a cyclooxygenase-2 selective inhibitor or its prodrug, and a pharmaceutically acceptable carrier, wherein the amount of the apical sodium co-dependent bile acid transport inhibitor and the amount of the cyclooxygenase-2 selective inhibitor together constitute a hypercholesterolemia-related condition effective amount or an inflammation-related condition effective amount of the said compounds. For example, one of the many embodiments of the present invention is a combination comprising therapeutic dosages of an ASBT inhibitor selected from Table 2 and a cyclooxygenase-2 selective inhibitor selected from Tables 4, 6 and 7A below. A preferred embodiment of the present invention is a combination comprising therapeutic dosages of a benzothiepine ASBT inhibitor and a tricyclic cyclooxygenase-2 selective inhibitor.

d. Cyclooxygenase Inhibitors

The present invention discloses that treatment of a subject with one or more ASBT inhibitors and one or more cyclooxygenase-2 selective inhibitors results in the prophylaxis and/or treatment of cardiovascular conditions and/or disorders. The method comprises treating the subject with an amount of an ASBT inhibitor and an amount of a cyclooxygenase-2 selective inhibitor or its prodrug, wherein the amount of the apical sodium co-dependent bile acid transport inhibitor and the amount of the cyclooxygenase-2 selective inhibitor together constitute a hypercholesterolemia-related condition effective amount or an inflammation-related condition effective amount of the said compounds.

For example, one of the many embodiments of the present invention is a combination therapy comprising a therapeutic amount of an ASBT inhibitor and a therapeutic amount of a cyclooxygenase inhibitor. The cyclooxygenase inhibitor can be, by way of example, a COX-2 nonselective inhibitor or a COX-2 selective inhibitor. Examples of COX-2 nonselective inhibitors include the well-known compounds aspirin, acetaminophen, indomethacin, sulindac, etodolac, mefenamic acid, tolmetin, ketorolac, diclofenac, ibuprofen, naproxen, fenoprofen, ketoprofen, oxaprozin, flurbiprofen, piroxicam, tenoxicam, phenylbutazone, apazone, or nimesulide,or a pharmaceutically acceptable salt or derivative or prodrug thereof. In a preferred embodiment of the invention the COX-2 nonselective inhibitor is selected from the group comprising aspirin, acetaminophen, indomethacin, ibuprofen, or naproxen.

In another embodiment of the invention the cyclooxygenase inhibitor can be a cyclooxygenase-2 selective inhibitor, for example, the COX-2 selective inhibitor meloxicam, Formula B-1 (CAS registry number 71125-38-7) or a pharmaceutically acceptable salt or derivative or prodrug thereof.

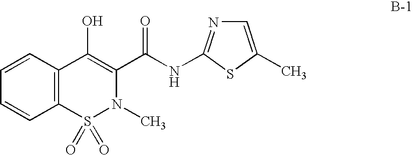

In yet another embodiment of the invention the cyclooxygenase-2 selective inhibitor is the COX-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3) or a pharmaceutically acceptable salt or derivative or prodrug thereof.

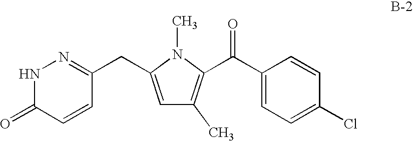

In a preferred embodiment of the invention the cyclooxygenase-2 selective inhibitor is a COX-2 selective inhibitor of the chromene structural class that is a substituted benzopyran or a substituted benzopyran analog selected from the group consisting of substituted benzothiopyrans, dihydroquinolines, or dihydronaphthalenes having the general Formula II shown below and possessing, by way of example and not limitation, the structures disclosed in Table 4, including the diastereomers, enantiomers, racemates, tautomers, salts, esters, amides and prodrugs thereof.

TABLE 4

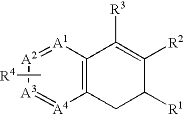

Examples of Chromene COX-2 Selective
Inhibitors as Embodiments

| Compound | |
|---|---|
| Number | Structural Formula |
| B-3 | 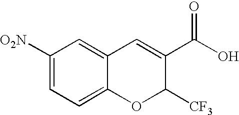 |
| | 6-Nitro-2-trifluoromethyl-2H-1- |
| | benzopyran-3-carboxylic acid |
| B-4 | 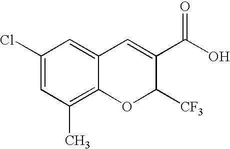 |
| | 6-Chloro-8-methyl-2-trifluoromethyl- |
| | 2H-1-benzopyran-3-carboxylic acid |
| B-5 | 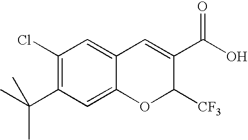 |
| | ((S)-6-Chloro-7-(1,1-dimethylethyl)-2-(trifluo |
| | romethyl-2H-1-benzopyran-3-carboxylic acid |
| B-6 | 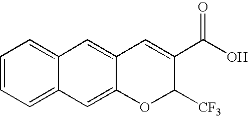 |
| | 2-Trifluoromethyl-2H-naphtho[2,3-b] |
| | pyran-3-carboxylic acid |
| B-7 | 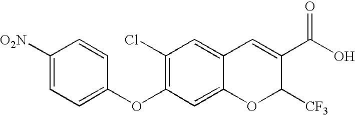 |
| | 6-Chloro-7-(4-nitrophenoxy)-2-(trifluoromethyl)-2H-1- |
| | benzopyran-3-carboxylic acid |
| B-8 | 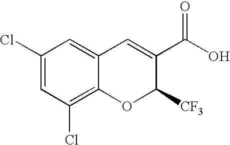 |
| | ((S)-6,8-Dichloro-2-(trifluoromethyl)- |
| | 2H-1-benzopyran-3-carboxylic acid |
| B-9 | 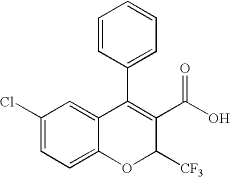 |
| | 6-Chloro-2-(trifluoromethyl-4-phenyl-2H- |
| | 1-benzopyran-3-carboxylic acid |
| B-10 | 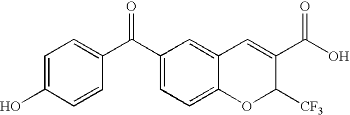 |
| | 6-(4-Hydroxybenzoyl)-2-(trifluoromethyl)- |
| | 2H-1-benzopyran-3-carboxylic acid |
| B-11 | 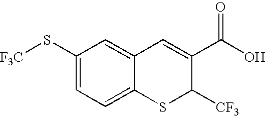 |
| | 2-(Trifluoromethyl)-6-[(trifluoromethyl)thiol]- |
| | 2H-1-benzothiopyran-3-carboxylic acid |
| B-12 | 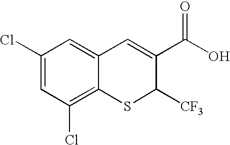 |
| | 6,8-Dichloro-2-trifluoromethyl-2H-1- |
| | benzothiopyran-3-carboxylic acid |
| B-13 | 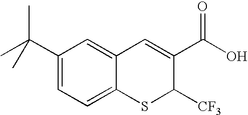 |
| | 6-(1,1-Dimethylethyl)-2-(trifluoromethyl)- |
| | 2H-1-benzothiopyran-3-carboxylic acid |
| B-14 | 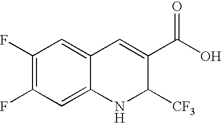 |
| | 6,7-Difluoro-1,2-dihydro-2-(trifluoro |
| | methyl)-3-quinolinecarboxylic acid |
| B-15 | 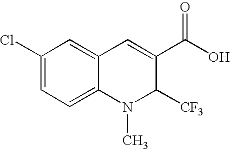 |
| | 6-Chloro-1,2-dihydro-1-methyl-2-(trifluoro |
| | methyl)-3-quinolinecarboxylic acid |
| B-16 | 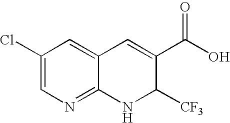 |
| | 6-Chloro-2-(trifluoromethyl)-1,2-dihydro |
| | [1,8]naphthyridine-3-carboxylic acid |
| B-17 | 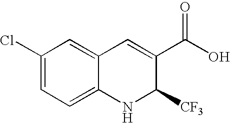 |
| | ((S)-6-Chloro-1,2-dihydro-2-(trifluoro |
| | methyl)-3-quinolinecarboxylic acid |

The individual patent documents referenced in Table 5 below describe the preparation of the aforementioned COX-2 inhibitors of Table 4 and are each herein incorporated by reference.

TABLE 5
References for Preparation of Chromene
COX-2 Inhibitors

| Compound Number | Patent Reference |
|---|---|
| B-3 | U.S. Pat. No. 6,077,850; example 37 |
| B-4 | U.S. Pat. No. 6,077,850; example 38 |
| B-5 | U.S. Pat. No. 6,077,850; example 68 |
| B-6 | U.S. Pat. No. 6,034,256; example 64 |
| B-7 | U.S. Pat. No. 6,077,850; example 203 |
| B-8 | U.S. Pat. No. 6,034,256; example 175 |
| B-9 | U.S. Pat. No. 6,077,850; example 143 |
| B-10 | U.S. Pat. No. 6,077,850; example 98 |
| B-11 | U.S. Pat. No. 6,077,850; example 155 |
| B-12 | U.S. Pat. No. 6,077,850; example 156 |
| B-13 | U.S. Pat. No. 6,077,850; example 147 |
| B-14 | U.S. Pat. No. 6,077,850; example 159 |
| B-15 | U.S. Pat. No. 6,034,256; example 165 |
| B-16 | U.S. Pat. No. 6,077,850; example 174 |
| B-17 | U.S. Pat. No. 6,034,256; example 172 |

In a more preferred embodiment of the invention the cycloxygenase-2 selective inhibitor is the substituted benzopyran (S)-6,8-dichloro-2-(trifluoromethyl)-2H-1-benzopyran-3-carboxylic acid, Formula B-8, or a pharmaceutically acceptable salt or derivative or prodrug thereof.

In a further preferred embodiment of the invention the cyclooxygenase inhibitor is selected from the class of tricyclic cyclooxygenase-2 selective inhibitors represented by the general structure of Formula III

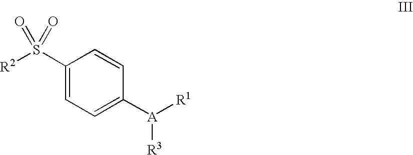

wherein A is a substituent selected from partially unsaturated or unsaturated heterocyclyl and partially unsaturated or unsaturated carbocyclic rings;

wherein R¹ is at least one substituent selected from heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is methyl or amino; and

wherein R³ is a radical selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt or derivative or prodrug thereof.

In a still more preferred embodiment of the invention the cyclooxygenase-2 selective inhibitor represented by the above Formula III is selected from the group of compounds, illustrated in Table 6, consisting of celecoxib (B-18), valdecoxib (B-19), deracoxib (B-20), rofecoxib (B-21), etoricoxib (MK-663; B-22), JTE-522 (B-23), or a pharmaceutically acceptable salt or derivative or prodrug thereof.

In an even more preferred embodiment of the invention the COX-2 selective inhibitor is selected from the group consisting of celecoxib, rofecoxib and etoricoxib.

TABLE 6
Examples of Tricyclic COX-2 Selective
Inhibitors as Embodiments

| Compound | |
|---|---|
| Number | Structural Formula |
| B-18 | 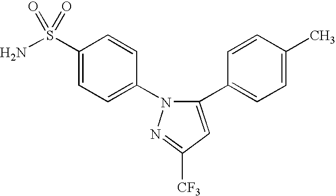 |
| B-19 | 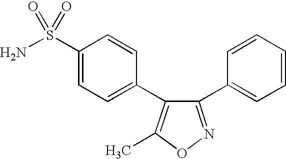 |
| B-20 | 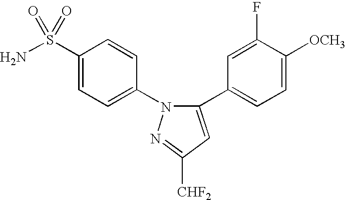 |
| B-21 | 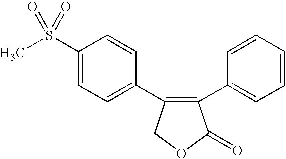 |
| B-22 | 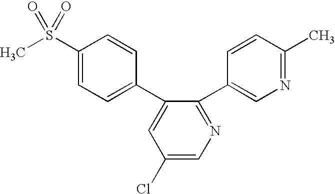 |
| B-23 | 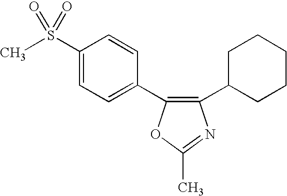 |

In another highly preferred embodiment of the invention parecoxib, B-24, which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

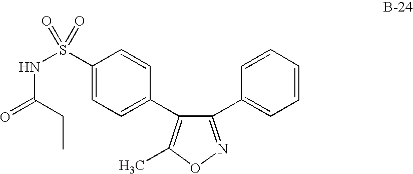

The individual patent documents referenced in Table 7 below describe the preparation of the aforementioned cyclooxygenase-2 selective inhibitors B-18 through B-24 and are each herein incorporated by reference.

TABLE 7
References for Preparation of Tricyclic
COX-2 Inhibitors and Prodrugs

| Compound Number | Patent Reference |
|---|---|
| B-18 | U.S. Pat. No. 5,466,823 |
| B-19 | U.S. Pat. No. 5,633,272 |
| B-20 | U.S. Pat. No. 5,521,207 |
| B-21 | U.S. Pat. No. 5,840,924 |
| B-22 | WO 98/03484 |
| B-23 | WO 00/25779 |
| B-24 | U.S. Pat. No. 5,932,598 |

Another embodiment of the present invention comprises a pharmaceutical combination containing an amount of an apical sodium co-dependent bile acid transport inhibitor and an amount of a cyclooxygenase inhibitor (e.g., cyclooxygenase-2 selective inhibitor) or its prodrug, and a pharmaceutically acceptable carrier, wherein the amount of the apical sodium co-dependent bile acid transport inhibitor and the amount of the cyclooxygenase inhibitor (e.g., cyclooxygenase-2 selective inhibitor) together constitute a hypercholesterolemia-related condition effective amount or an inflammation-related condition effective amount of the said compounds. For example, one of the many embodiments of the present invention is a combination comprising therapeutic dosages of an ASBT inhibitor selected from the aforementioned Table 2 and a COX-2 selective inhibitor selected from the aforementioned Tables 4, 6 and 7A. A preferred embodiment of the present invention is a combination containing therapeutic dosages of a benzothiepine ASBT inhibitor and a tricyclic COX-2 selective inhibitor.

Another preferred embodiment of the present invention is a combination containing therapeutic dosages of an ASBT inhibitor selected from Table 2 and a COX-2 selective inhibitor selected from Table 7A below.

TABLE 7A

| Component 2 | Name and/or Structure (COX-2 Selective Inhibitor) |
|---|---|
| D-1 | 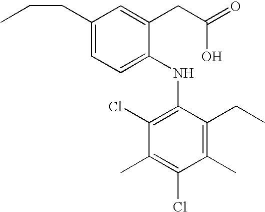 |
| | [2-(2,4-Dichloro-6-ethyl-3,5-dimethyl-phenylamino)- |
| | 5-propyl-phenyl]-acetic acid; |
| D-2 | 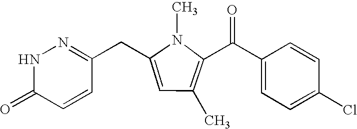 |
| | 6-[[5- (4-chlorobenzoyl)-1,4-dimethyl-1H-pyrrol-2- |
| | yl]methyl]-3(2H)-pyridazinone or RS 57067; |
| D-3 | 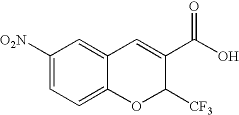 |
| | 6-Nitro-2 -trif1uoromethyl-2H-1- |
| | benzopyran-3-carboxylic acid; |
| D-4 | 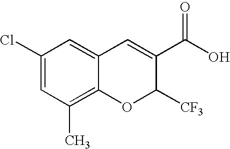 |
| | 6-Chloro-8-methyl-2-trifluoromethyl- |
| | 2H-1-benzopyran-3-carboxylic acid; |
| D-5 | 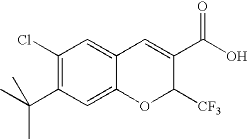 |
| | ((S)-6-Chloro-7-(1,1-dimethylethyl)-2-(trifluo |
| | romethyl-2H-1-benzopyran-3-carboxylic acid; |
| D-6 | 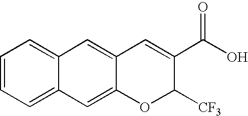 |
| | 2-Trifluoromethyl-2H-naphtho[2,3-b] |
| | pyran-3-carboxylic acid; |
| D-7 | 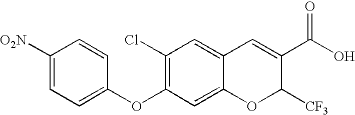 |
| | 6-Chloro-7-(4-nitrophenoxy)-2-(trifluoromethyl)-2H-1- |
| | benzopyran-3-carboxylic acid; |
| D-8 | 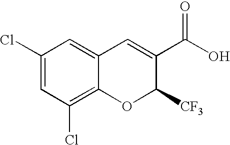 |
| | ((S)-6,8-Dichloro-2-(trifluoromethyl)- |
| | 2H-1-benzopyran-3-carboxylic acid; |
| D-9 | 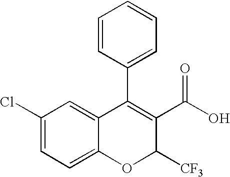 |
| | 6-Chloro-2-(trifluoromethyl)-4-phenyl-2H- |
| | 1-benzopyran-3-carboxylic acid; |
| D-10 | 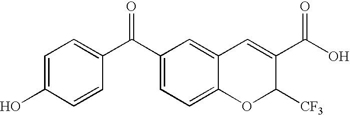 |
| | 6-(4-Hydroxybenzoyl)-2-(trifluoromethyl)- |
| | 2H-1-benzopyran-3-carboxylic acid; |
| D-11 | 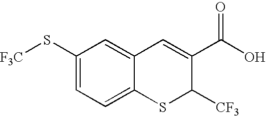 |
| | 2-(Trifluoromethyl)-6-[(trifluorornethyl)thio]- |
| | 2H-1-benzothiopyran-3-carhoxylic acid; |
| D-12 | 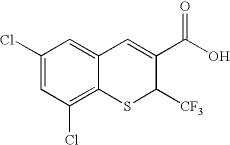 |
| | 6 8-Dichloro-2-trifluoromethyl-2H-1- |
| | benzothiopyran-3-carboxylic acid; |
| D-13 | 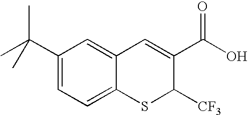 |
| | 6-(1,1-Dimethylethyl)-2-(trifluoromethyl)- |
| | 2H-1-benzothiopyran-3-carboxylic acid; |
| D-14 | 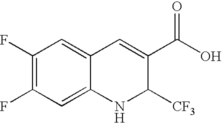 |
| | 6,7-Difluoro-1,2-dihydro-2-(trifluoro |
| | methyl)-3-quinolinecarboxylic acid; |
| D-15 | 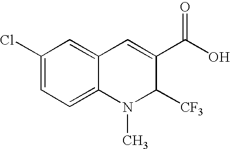 |
| | 6-Chloro-1,2-dihydro-1-methyl-2-(trifluoro |
| | methyl)-3-quinolinecarboxylic acid; |
| D-16 | 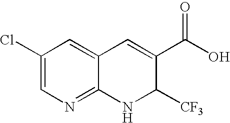 |
| | 6-Chloro-2-(trifluoromethyl)-1,2-dihydro |
| | [1,8]naphthyridine-3-carboxylic acid; |
| D-17 | 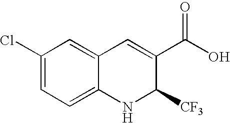 |
| | ((S)-6-Chloro-1,2-dihydro-2-(trifluoro |
| | methyl)-3-quinolinecarboxylic acid; |
| D-18 | 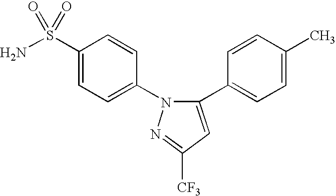 |
| | celecoxib; |
| D-19 | 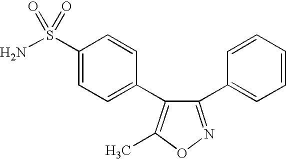 |
| | valdecoxib; |
| D-20 | 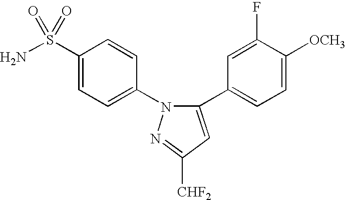 |
| | deracoxib; |
| D-21 | 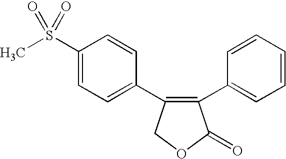 |
| | rofecoxib; |
| D-22 | 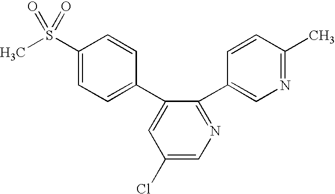 |
| | etoricoxib; |
| D-23 | 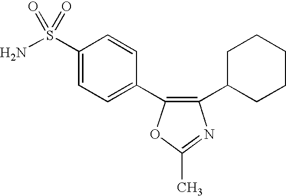 |
| | JTE-522 |
| D-24 | 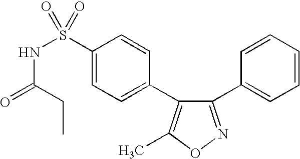 |
| | parecoxib; |
| D-25 | 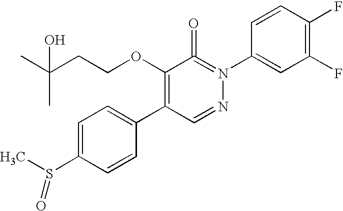 |
| | ABT-963 |
| D-26 | 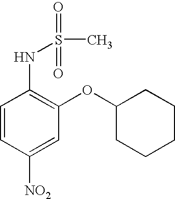 |
| | N-(2-Cyclohexyloxy-4-nitro-phenyl)-methanesulfonamide |
| | or NS-398; |
| D-27 | 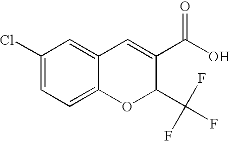 |
| | 6-chloro-2-trifluoromethyl-2H-1-benzopyran-3-carboxylic acid; |
| D-28 | 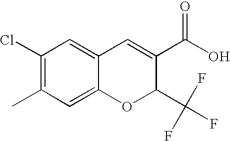 |
| | 6-chloro-7-methyl-2-trifluoromethyl-2H-1-benzopyran-3-carboxylic acid; |
| D-29 | 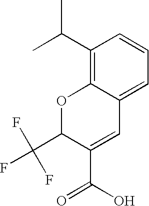 |
| | 8-(1-methylethyl)-2-trifluoromethyl-2H-1-benzopyran-3-carboxylic acid; |
| D-30 | 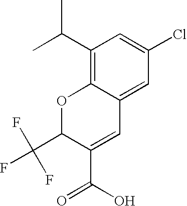 |
| | 6-chloro-8-(1-methylethyl)-2-fluoromethyl-2H-1-benzopyran carboxylic acid; |
| D-31 | [INSERT STRUCTURE] |
| | 2-trifluoromethyl-3H-naphthopyran-3-carboxylic acid; |
| D-32 | 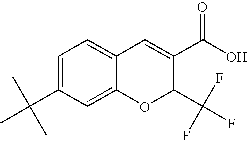 |
| | 7-(1,1-dimethylethyl)-2-trifluoromethyl-2H-1-benzopyran-3-carboxylic acid; |
| D-33 | 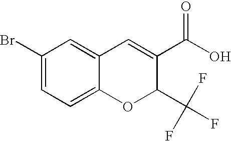 |
| | 6-bromo-2-trifluoromethyl-2H-1-benzopyran-3-carboxylic acid; |
| D-34 | 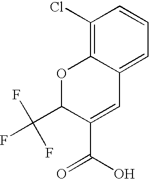 |
| | 8-chloro-2-trifluoromethyl-2H-1-benzopyran-3-carboxylic acid; |
| D-35 | 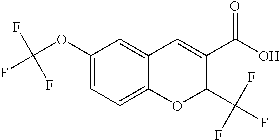 |
| | 6-trifluoromethoxy-2-trifluoromethyl-2H-1-benzopyran-3-carboxylic acid; |
| D-36 | 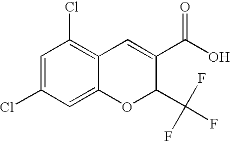 |
| | 5,7-dichloro-2-trifluoromethyl-2H-1-benzopyran-3-carboxylic acid; |
| D-37 | 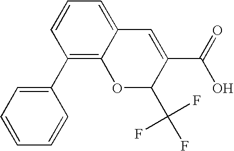 |
| | 8-phenyl-2-trifluoromethyl-2H-1-benzopyran-3-carboxylic acid; |
| D-38 | 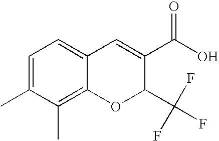 |
| | 7,8-dimethyl-2-trifluoromethyl-2H-1-benzopyran-3-carboxylic acid; |
| D-39 | 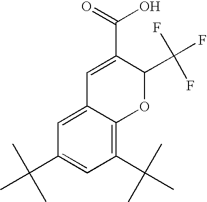 |
| | 6,8-bis(dimethylethyl)-2-trifluoromethyl-2H-1-benzopyran-3-carboxylic acid; |
| D-40 | 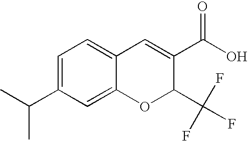 |
| | 7-(1-methylethyl)-2-trifluoromethyl-2H-1-benzopyran-3-carboxylic acid; |
| D-41 | 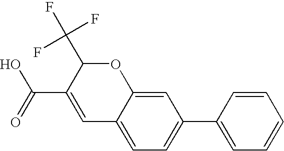 |
| | 7-phenyl-2-trifluoromethyl-2H-1-benzopyran-3-carboxylic acid; |
| D-42 | 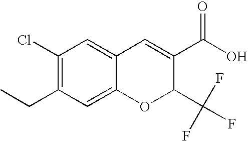 |
| | 6-chloro-7-ethyl-2-trifluoromethyl-2H-1-benzopyran-3-carboxylic acid |
| D-43 | 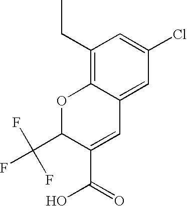 |
| | 6-chloro-8-ethyl-2-trifluoromethyl-2H-1-benzopyran-3-carboxylic acid; |
| D-44 | 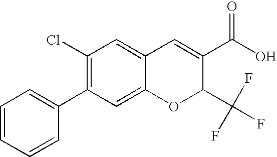 |
| | 6-chloro-7-phenyl-2-trifluoromethyl-2H-1-benzopyran-3-carboxylic acid; |
| D-45 | 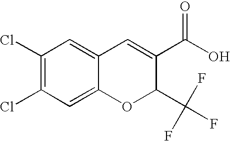 |
| | 6,7-dichloro-2-trifluoromethyl-2H-1-benzopyran-3-carboxylic acid; |
| D-46 | 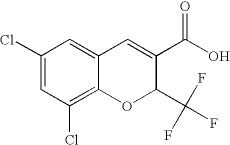 |
| | 6,8-dichloro-2-trifluoromethyl-2H-1-benzopyran-3-carboxylic acid; |
| D-47 | 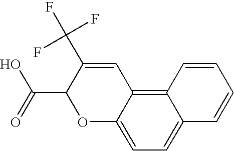 |
| | 2-trifluoromethyl-3H-naptho[2,1-b]pyran-3-carboxylic acid; |
| D-48 | 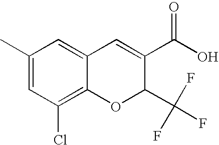 |
| | 8-chloro-6-methyl-2-trifluoromethy1-2H-1-benzopyran-3-carboxylic acid; |
| D-49 | 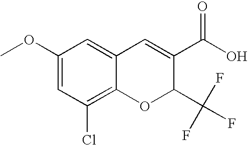 |
| | 8-chloro-6-methoxy-2-trifluoromethyl-2H-1-benzopyran-3-carboxylic acid; |
| D-50 | 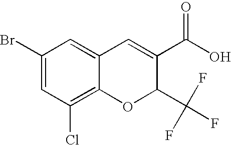 |
| | 6-bromo-8-chloro-2-trifluoromethyl-2H-1-benzopyran-3-carboxylic acid; |
| D-51 | 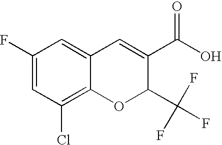 |
| | 8-bromo-6-fluoro-2-trifluoromethyl-2H-1-benzopyran-3-carboxylic acid; |
| D-52 | 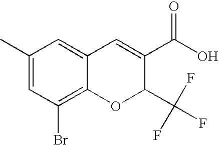 |
| | 8-bromo-6-methyl-2-trifluoromethyl-2H-1-benzopyran-3-carboxylic acid; |
| D-53 | 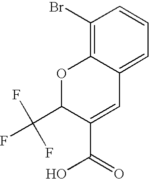 |
| | 8-bromo-5-fluoro-2-trifluoromethyl-2H-1-benzopyran-3-carboxylic acid; |
| D-54 | 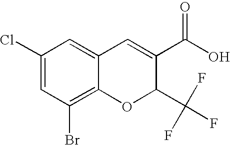 |
| | 6-chloro-8-fluoro-2-trifluoromethyl-2H-1-benzopyran-3-carboxylic acid; |
| D-55 | 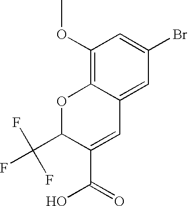 |
| | 6-bromo-8-methoxy-2-trifluoromethyl-2H-1-benzopyran-3-carboxylic acid; |
| D-56 | 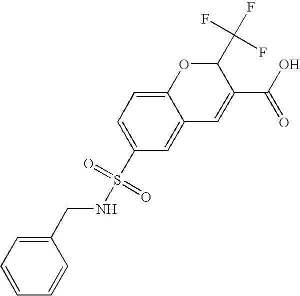 |
| | 6-[[(phenylmethyl)amino]sulfonyl]-2-trifluoromethyl-2H-1-benzopyran- |
| | 3-carboxylic acid; |
| D-57 | 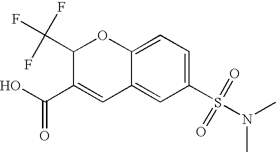 |
| | 6-[(dimethylamino)sulfonyl]-2-trifluoromethyl-2H-1-benzopyran- |
| | 3-carboxylic acid; |
| D-58 | 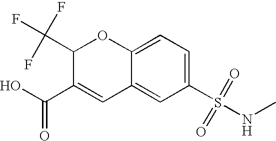 |
| | 6-[(methylamino)sulfonyl]-2-trifluoromethyl-2H-1-benzopyran- |
| | 3-carboxylic acid; |
| D-59 | 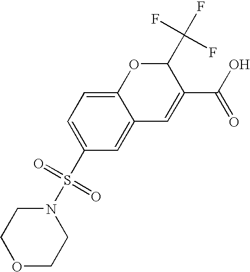 |
| | 6-[(4-morpholino)sulfonyl]-2-trifluoromethyl-2H-1-benzopyran- |
| | 3-carboxylic acid; |
| D-60 | 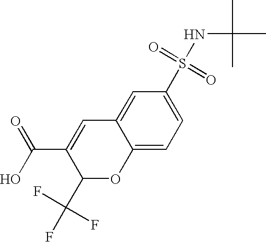 |
| | 6-[(1,1-dimethylethyl)aminosulfonyl]-2-trifluoromethyl-2H-1-benzopyran- |
| | 3-carboxylic acid; |
| D-61 | 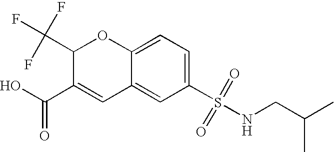 |
| | 6-[(2-methylpropyl)aminosulfonyl]-2-trifluoromethyl-2H-1-benzopyran- |
| | 3-carboxylic acid; |
| D-62 | 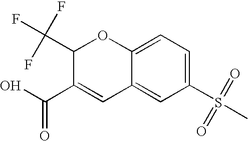 |
| | 6-methylsulfonyl-2-trifluoromethyl-2H-1-benzopyran-3-carboxylic acid; |
| D-63 | 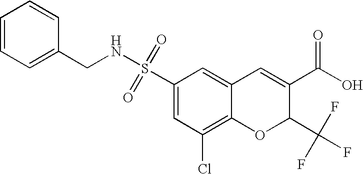 |
| | 8-chloro-6-[[(phenylmethyl)amino]sulfonyl]-2-trifluoromethyl-2H- |
| | 1-benzopyran-3-carboxylic acid; |
| D-64 | 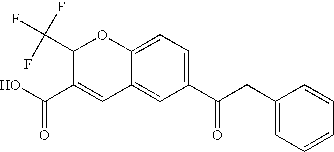 |
| | 6-phenylacetyl-2-trifluoromethyl-2H-1-benzopyran-3-carboxylic acid; |
| D-65 | 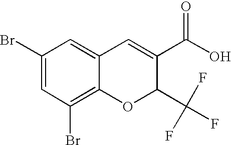 |
| | 6,8-dibromo-2-trifluoromethyl-2H-1-benzopyran-3-carboxylic acid; |
| D-66 | 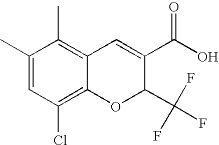 |
| | 8-chloro-5,6-dimethyl-2-trifluoromethyl-2H-1-benzopyran-3-carboxylic acid; |
| D-67 | 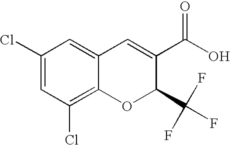 |
| | 6,8-dichloro-(S)-2-trifluoromethyl-2H-1-benzopyran-3-carboxylic acid; |
| D-68 | 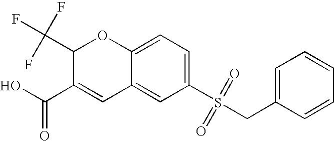 |
| | 6-benzylsulfonyl-2-trifluoromethyl-2H-1-benzopyran-3-carboxylic acid; |
| D-69 | 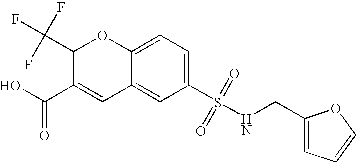 |
| | 6-[[N-(2-furylmethyl)amino]sulfonyl]-2-trifluoromethyl-2H-1-benzopyran- |
| | 3-carboxylic acid; |
| D-70 | 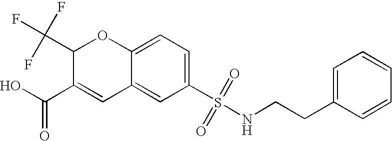 |
| | 6-[[N-(2-phenylethyl)amino]sulfonyl]-2-trifluoromethyl-2H-1-benzopyran- |
| | 3-carboxylic acid; |
| D-71 | 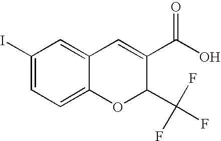 |
| | 6-iodo-2-trifluoromethyl-2H-1-benzopyran-3-carboxylic acid; |
| D-72 | 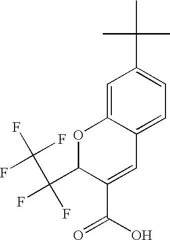 |
| | 7-(1,1-dimethylethyl)-2-pentafluoroethyl-2H-1-benzopyran- |
| | 3-carboxylic acid; |
| D-73 | 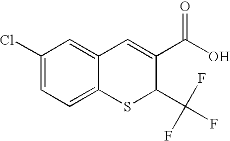 |
| | 6-chloro-2-trifluoromethyl-2H-1-benzothiopyran-3-carboxylic acid; |
| D-74 | 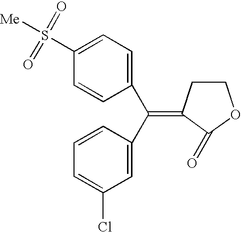 |
| | BMS-347070 |
| D-75 | 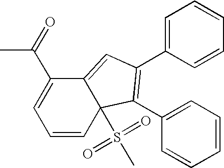 |
| | 8-acetyl-3-(4-fluorophenyl)-2-(4-methylsulfonyl)phenyl- |
| | imidazo(1,2-a)pyridine; |
| D-76 | 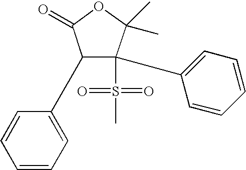 |
| | 5,5-dimethyl-4-(4-methylsulfonyl)phenyl-3-phenyl-2-(5H)-furanone; |
| D-77 | 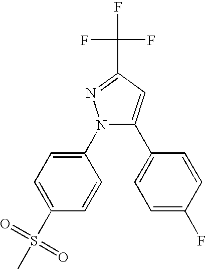 |
| | 5-(4-fluorophenyl)-1-[4-(methylsulfonyl)pheuyl]-3-(trifluoromethyl)pyrazole; |
| D-78 | 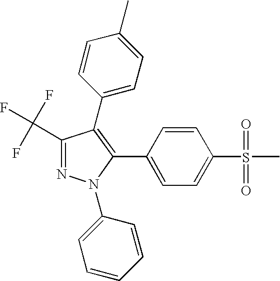 |
| | 4-(4-fluorophenyl)-5-[4-(methylsulfonyl)phenyl]-1-phenyl- |
| | 3-(trifluoromethyl)pyrazole; |
| D-79 | 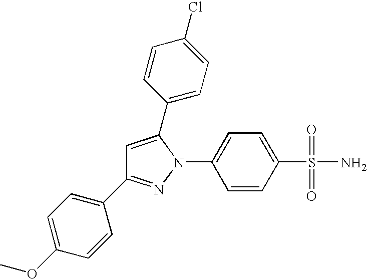 |
| | 4-(5-(4-chlorophenyl)-3-(4-methoxyphenyl)-1H-pyrazol-1- |
| | yl)benzenesulfonamide; |
| D-80 | 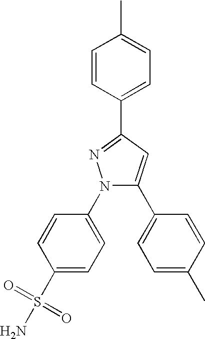 |
| | 4-(3,5-bis(4-methylphenyl)-1H-pyrazol-1-yl)benzenesulfonamide; |
| D-81 | 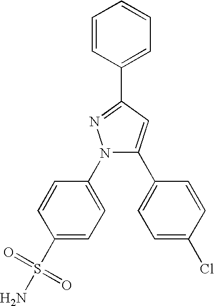 |
| | 4-(5-(4-chlorophenyl)-3-phenyl-1H-pyrazol-1-yl)benzenesulfonamide; |
| D-82 | 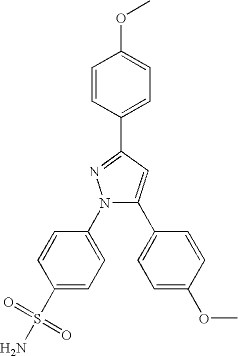 |
| | 4-(3,5-bis(4-methoxyphenyl)-1H-pyrazol-1-yl)benzenesulfonamide; |
| D-83 | 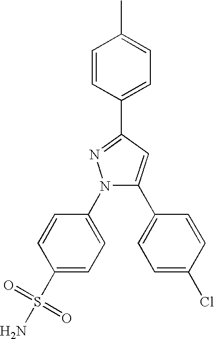 |
| | 4-(5-(4-chlorophenyl)-3-(4-methylphenyl)-1H-pyrazol-1- |
| | yl)benzenesulfonamide; |
| D-84 | 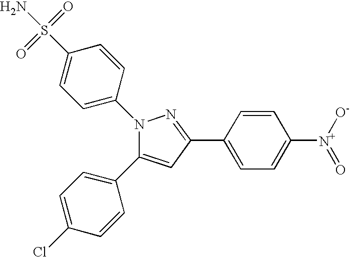 |
| | 4-(5-(4-chlorophenyl)-3-(4-nitrophenyl)-1H-pyrazol-1- |
| | yl)benzenesulfonamide; |
| D-85 | 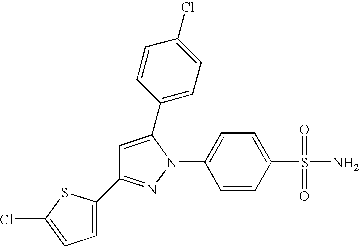 |
| | 4-(5-(4-chlorophenyl)-3-(5-chloro-2-thienyl)-1H-pyrazol-1- |
| | yl)benzenesulfonamide; |
| D-86 | 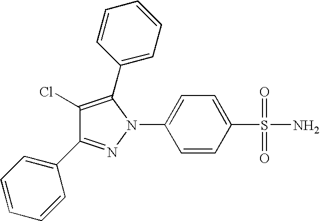 |
| | 4-(4-chloro-3,5-diphenyl-1H-pyrazol-1-yl)benzenesulfonamide; |
| D-87 | 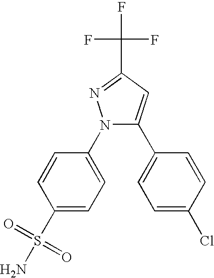 |
| | 4-[5-(4-chlorophenyl)-3-(trifluoromethyl)-1H-pyrazol-1- |
| | yl]benzenesulfonamide; |
| D-88 | 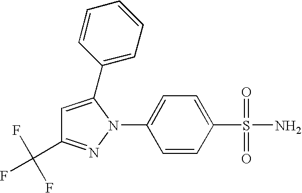 |
| | 4-[5-phenyl-3-(trifluoromethyl)-1H-pyrazol-1-yl]benzenesulfonamide; |
| D-89 | 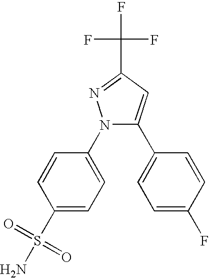 |
| | 4-[5-(4-fluorophenyl)-3-(trifluoromethyl)-1H-pyrazol-1- |
| | yl]benzenesulfonamide; |
| D-90 | 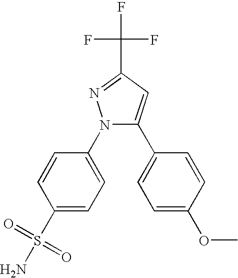 |
| | 4-[5-(4-methoxyphenyl)-3-(trifluoromethyl)-1H-pyrazol-1- |
| | yl]benzenesulfonamide; |
| D-91 | 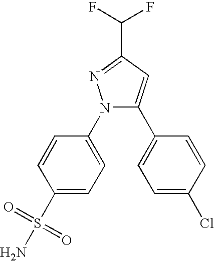 |
| | 4-[5-(4-chlorophenyl)-3-(difluoromethyl)-1H-pyrazol-1- |
| | yl]benzenesulfonamide; |
| D-92 | 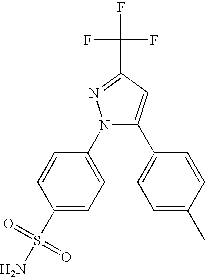 |
| | 4-[5-(4-methylphenyl)-3-(trifluoromethyl)-1H-pyrazol-1- |
| | yl]benzenesulfonamide; |
| D-93 | 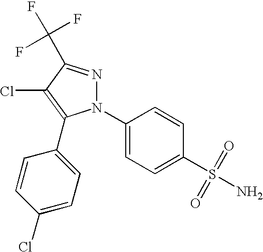 |
| | 4-[4-chloro-5-(4-chloropheny1)-3-(trifluoromethy1)-1H-pyrazol-1- |
| | yl]benzenesulfonamide; |
| D-94 | 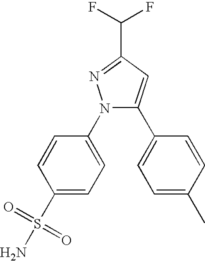 |
| | 4-[3-(difluoromethyl)-5-(4-methylphenyl)-1H-pyrazol-1- |
| | yl]benzenesulfonamide; |
| D-95 | 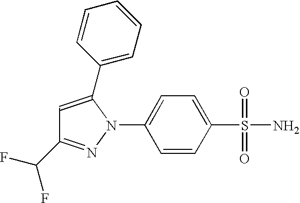 |
| | 4-[3-(difluoromethyl)-5-phenyl-1H-pyrazol-1-yl]benzenesulfonamide; |
| D-96 | 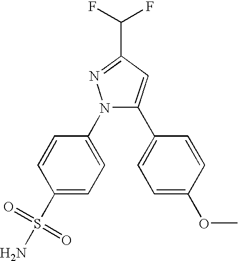 |
| | 4-[3-(difluoromethyl)-5-(4-methoxyphenyl)-1H-pyrazol-1- |
| | yl]benzenesulfonamide; |
| D-97 | 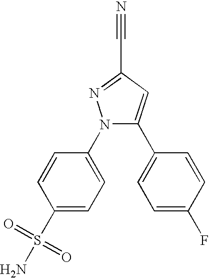 |
| | 4-[3-cyano-5-(4-fluorophenyl)-1H-pyrazol-1-yl]benzenesulfonamide; |
| D-98 | 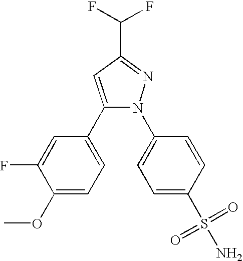 |
| | 4-[3-(difluoromethyl)-5-(3-fluoro-4-methoxyphenyl)-1H-pyrazol-1- |
| | yl]benzenesulfonamide; |
| D-99 | 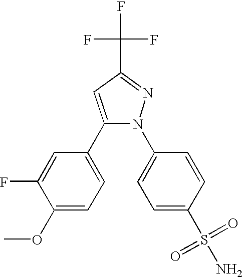 |
| | 4-[5-(3-fluoro-4-methoxyphenyl)-3-(trifluoromethyl)-1H-pyrazol-1- |
| | yl]benzenesulfonamide; |
| D-100 | 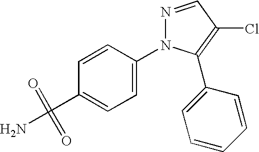 |
| | 4-[4-chloro-5-phenyl-1H-pyrazol-1-yl]benzenesulfonamide; |
| D-101 | 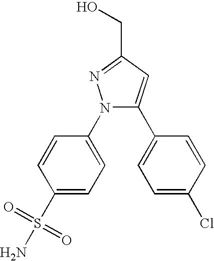 |
| | 4-[5-(4-chlorophenyl)-3-(hydroxymethyl)-1H-pyrazol-1- |
| | yl]benzenesulfonamide; |
| D-102 | 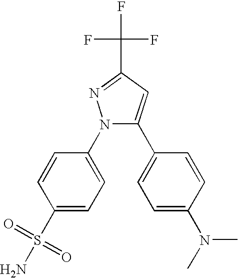 |
| | 4-[5-(4-(N,N-dimethylamino)phenyl)-3-(trifluoromethyl)-1H-pyrazol-1- |
| | yl]benzenesulfonaniide; |
| D-103 | 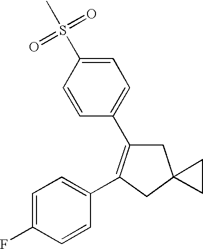 |
| | 5-(4-fluorophenyl)-6-[4-(methylsulfonyl)phenyl]spiro[2.4]hept-5-ene; |
| D-104 | 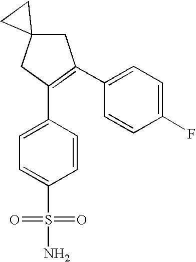 |
| | 4-[6-(4-fluorophenyl)spiro[2.4]hept-5-en-5-yl]benzenesulfonamide; |
| D-105 | 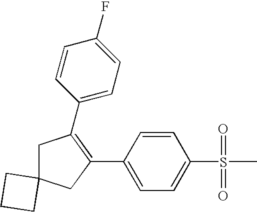 |
| | 6-(4-fluoropheny1)-7-[4-(methylsulfonyl)phenyl]spiro[3.4]oct-6-ene; |
| D-106 | 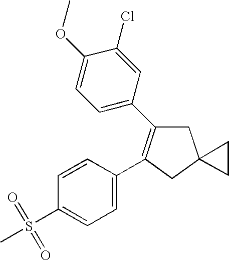 |
| | 5-(3-chloro-4-methoxyphenyl)-6-[4- |
| | (methylsulfonyl)phenyl]spiro[2.4]hept-5-ene; |
| D-107 | 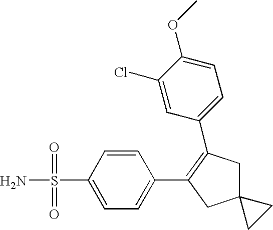 |
| | 4-[6-(3-chloro-4-methoxyphenyl)spiro[2.4]hept-5-en-5- |
| | yl]benzenesulfonamide; |
| D-108 | 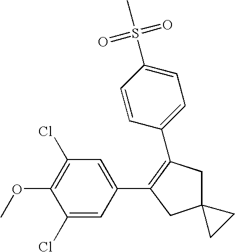 |
| | 5-(3,5-dichloro-4-methoxyphenyl)-6-[4- |
| | (methylsulfonyl)phenyl]spiro[2.4]hept-5-ene; |
| D-109 | 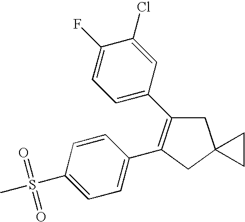 |
| | 5-(3-chloro-4-fluorophenyl)-6[4- |
| | (methylsulfonyl)phenyl]spiro[2.4]hept-5-ene; |
| D-110 | 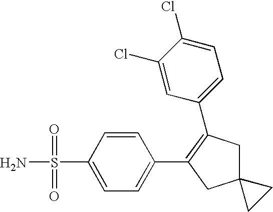 |
| | 4-[6-(3,4-dichlorophenyl)spiro[2.4]hept-5-en-5-yl]benzenesulfonamide; |
| D-111 | 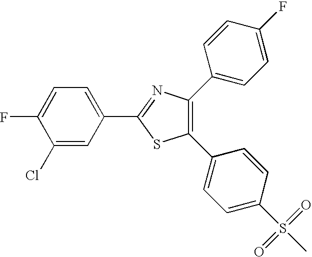 |
| | 2-(3-chloro-4-fluorophenyl)4-(4-fluorophenyl)-5- |
| | (4-methylsulfonylphenyl)thiazole; |
| D-112 | 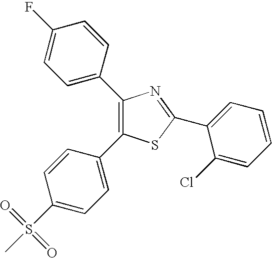 |
| | 2-(2-chlorophenyl)-4-(4-fluorophenyl)-5-(4-methylsulfonylphenyl)thiazole; |
| D-113 | 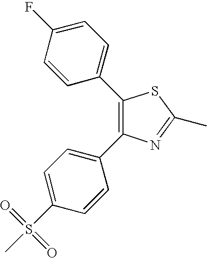 |
| | 5-(4-fluorophenyl)-4-(4-methylsulfonylphenyl)-2-methylthiazole; |
| D-114 | 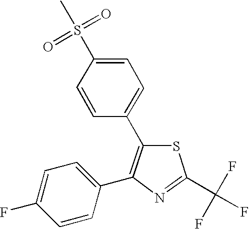 |
| | 4-(4-fluorophenyl)-5-(4-methylsulfonylphenyl)-2-trifluoromethylthiazole; |
| D-115 | 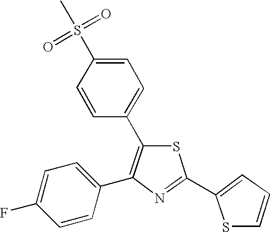 |
| | 4-(4-fluorophenyl)-5-(4-methylsulfonylphenyl)-2-(2-thienyl)thiazole; |
| D-116 | 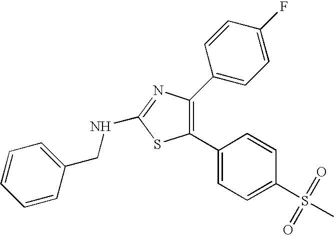 |
| | 4-(4-fluorophenyl)-5-(4-methylsulfonylphenyl)-2-benzylaminothiazole; |
| D-117 | 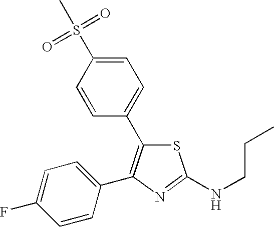 |
| | 4-(4-fluorophenyl)-5-(4-methylsulfonylphenyl)-2-(1-propylamino)thiazole; |
| D-118 | 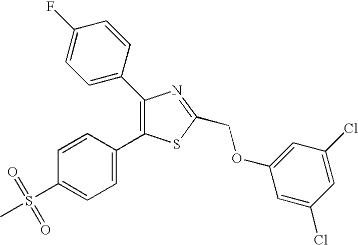 |
| | 2-((3,5-dichlorophenoxy)methyl)-4-(4-fluorophenyl)-5- |
| | [4-(methylsulfonyl)phenyl]thiazole; |
| D-119 | 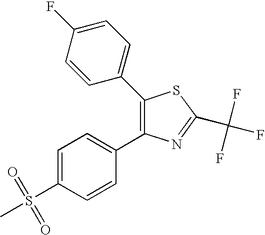 |
| | 5-(4-fluorophenyl)-4-(4-methylsulfonylphenyl)-2-trifluoromethylthiazole; |
| D-120 | 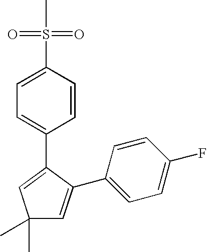 |
| | 1-methylsulfonyl-4-[1,1-dimethyl-4-(4-fluorophenyl)cyclopenta- |
| | 2,4-dien-3-yl]benzene; |
| D-121 | 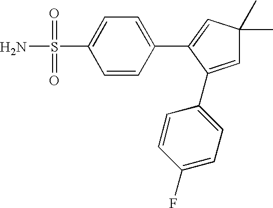 |
| | 4-[4-(4-fluorophenyl)-1,1-dimethylcyclopenta-2,4-dien-3- |
| | yl]benzenesulfonamide; |
| D-122 | 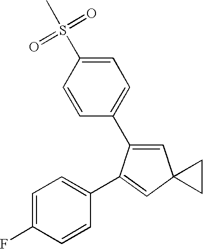 |
| | 5-(4-fluorophenyl)-6-[4-(methylsulfonyl)phenyl[spiro[2.4]hepta-4,6-diene; |
| D-123 | 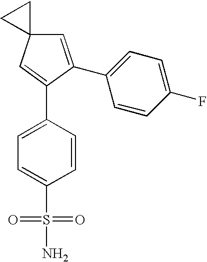 |
| | 4-[6-(4-fluorophenyl)spiro[2.4]hepta-4,6-dien-5-yl]benzenesulfonamide; |
| D-124 | 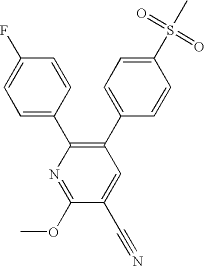 |
| | 6-(4-fluorophenyl)-2-methoxy-5-[4-(methylsulfonyl)phenyl]-pyridine- |
| | 3-carbonitrile; |
| D-125 | 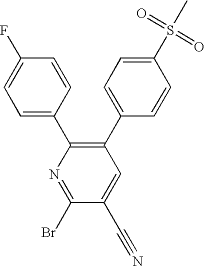 |
| | 2-bromo-6-(4-fluorophenyl)-5-[4-(methylsulfonyl)phenyl]-pyridine- |
| | 3-carbonitrile; |
| D-126 | 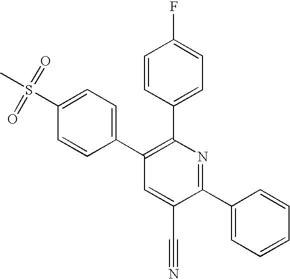 |
| | 6-(4-fluorophenyl)-5-[4-(methylsulfonyl)phenyl]-2-phenyl-pyridine- |
| | 3-carbonitrile; |
| D-127 | 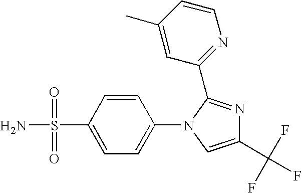 |
| | 4-[2-(4-methylpyridin-2-yl)-4-(trifluoromethyl)-1H-imidazol-1- |
| | yl]benzenesulfonamide; |
| D-128 | 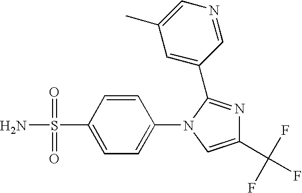 |
| | 4-[2-(5-methylpyridin-3-yl)-4-(trifluoromethyl)-1H-imidazol-1- |
| | yl]benzenesulfonamide; |
| D-129 | 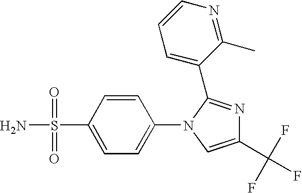 |
| | 4-[2-(2-methylpyridin-3-yl)-4-(trifluoromethyl)-1H-imidazol-1- |
| | yl]benzenesulfonamide; |
| D-130 | 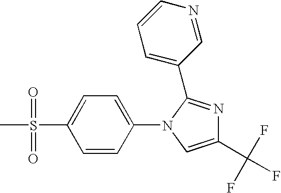 |
| | 3-[1-[4-(methylsulfonyl)phenyl]-4-(trifluoromethyl)-1H-imidazol-2- |
| | yl]pyridine; |
| D-131 | 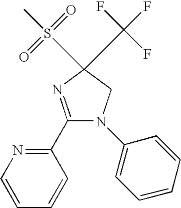 |
| | 2-[1-[4-(methylsulfonyl)phenyl-4-(trifluoromethyl)]-1H-imidazol-2- |
| | yl]pyridine; |
| D-132 | 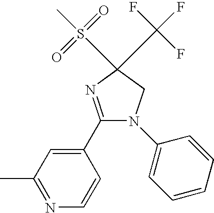 |
| | 2-methyl-4-[1-[4-(methylsulfonyl)phenyl-4-(trifluoromethyl)]-1H-imidazol-2- |
| | yl]pyridine; |
| D-133 | 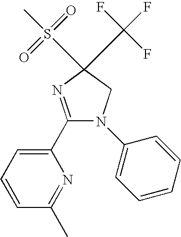 |
| | 2-methyl-6-[1-[4-(methylsulfonyl)phenyl-4-(trifluoromethyl)]-1H-imidazol-2- |
| | y]pyridine; |
| D-134 | 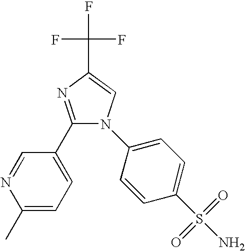 |
| | 4-[2-(6-methylpyridin-3-yl)-4-(trifluoromethyl)-1H-imidazol-1- |
| | yl]benzenesulfonamide; |
| D-135 | 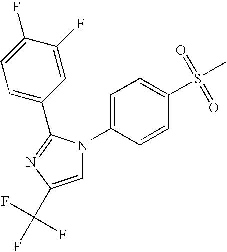 |
| | 2-(3,4-difluorophenyl)-1-[4-(methylsulfonyl)phenyl]- |
| | 4-(trifluoromethyl)-1H-imidazole; |
| D-136 | 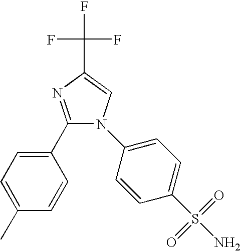 |
| | 4-[2-(4-methylphenyl)-4-(trifluoromethyl)-1H-imidazol-1- |
| | yl]benzenesulfonamide; |
| D-137 | 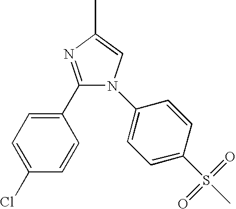 |
| | 2-(4-chlorophenyl)-1-[4-(methylsulfonyl)phenyl]-4-methyl-1H-imidazole; |
| D-138 | 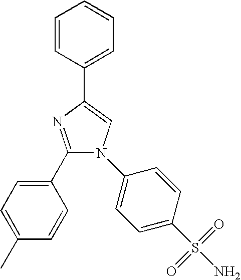 |
| | 2-(4-chlorophcnyl)-1-[4-(methylsulfonyl)phenyl]-4-phenyl-1H-imidazole; |
| D-139 | 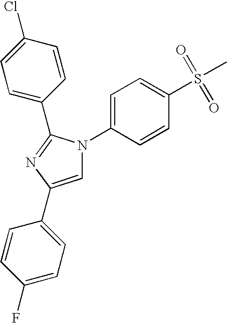 |
| | 2-(4-chlorophenyl)-4-(4-fluorophenyl)-1[4-(methylsulfonyl)phenyl]- |
| | 1H-imidazole; |
| D-140 | 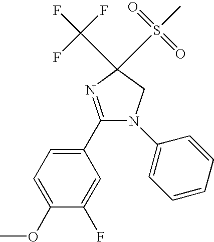 |
| | 2-(3-fluoro-4-methoxyphenyl)-1-[4-(methylsulfonyl)phenyl- |
| | 4(trifluoromethyl)]-1H-imidazole; |
| D-141 | 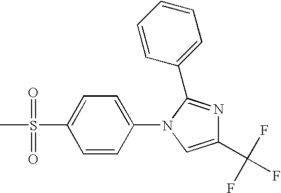 |
| | 1-[4-(methylsulfonyl)phenyl]-2-phenyl-4-trifluoromethyl-1H-imidazole; |
| D-142 | 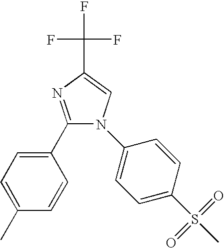 |
| | 2-(4-methylphenyl)-1-[4-(methylsulfonyl)phenyl]4-trifluoromethyl- |
| | 1H-imidazole; |
| D-143 | 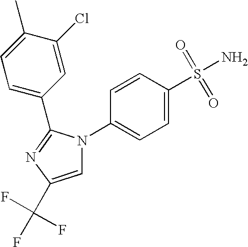 |
| | 4-[2-(3-chloro-4-methylphenyl)-4-(trifluoromethyl)-1H-imidazol-1- |
| | yl]benzenesulfonamide; |
| D-144 | 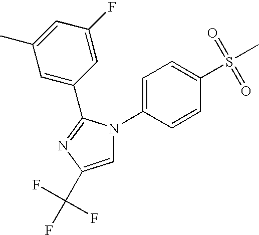 |
| | 2-(3-fluoro-5-methylphenyl)-1-[4-(methylsulfonyl)phenyl]- |
| | 4-(trifluoromethyl)-1H-imidazole; |
| D-145 | 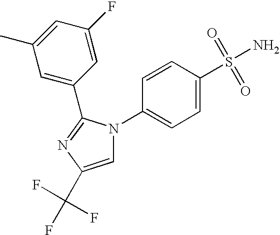 |
| | 4-[2-(3-fluoro-5-methylphenyl)-4-(trifluoromethyl)-1H-imidazol-1- |
| | yl]benzenesulfonamide; |
| D-146 | 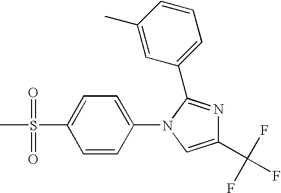 |
| | 2-(3-methylphenyl)-1-[4-(methylsulfonyl)phenyl]-4-trifluoromethyl- |
| | 1H-imidazole; |
| D-147 | 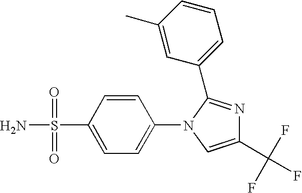 |
| | 4-[2-(3-methylphenyl)-4-trifluoromethyl-1H-imidazol-1- |
| | yl]benzenesulfonamide; |
| D-148 | 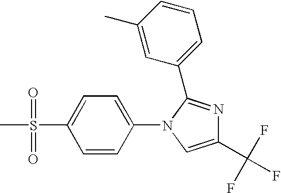 |
| | 1-[4-(methylsulfonyl)phenyl]-2-(3-chlorophenyl)-4-trifluoromethyl- |
| | 1H-imidazole; |
| D-149 | 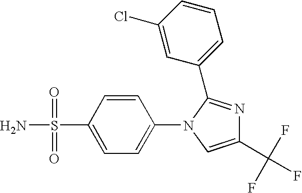 |
| | 4-[2-(3-chlorophenyl)-4-trifluoromethyl-1H-imidazol-1- |
| | yl]benzenesulfonamide; |
| D-150 | 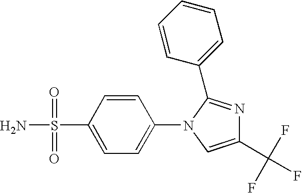 |
| | 4-[2-phenyl-4-trifluoromethyl-1H-imidazol-1-yl]benzenesulfonamide; |
| D-151 | 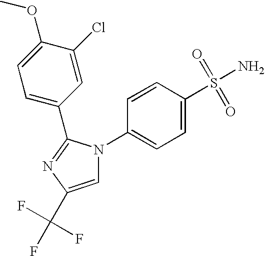 |
| | 4-[2-(4-methoxy-3-chlorophenyl)-4-trifluoromethyl-1H-imidazol-1- |
| | yl]benzenesulfonamide; |
| D-152 | 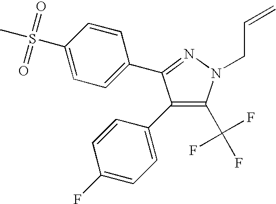 |
| | 1-allyl-4-(4-fluorophenyl)-3-[4-(methylsulfonyl)phenyl]-5-(trifluoromethyl)- |
| | 1H-pyrazole; |
| D-153 | 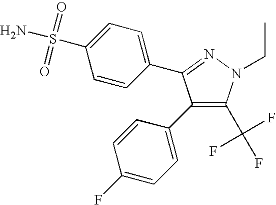 |
| | 4-[1-ethyl-4-(4-fluorophenyl)-5-(trifluoromethyl)-1H-pyrazol-3- |
| | yl]benzenesulfonamide; |
| D-154 | 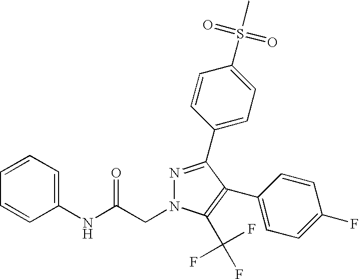 |
| | N-phenyl-[4-(4-fluorophenyl)-3-[4-(methylsulfonyl)phenyl]- |
| | 5-(trifluoromethyl)-1H-pyrazol-1-yl]acetamide; |
| D-155 | 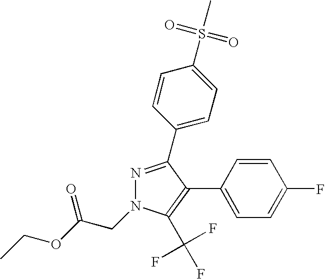 |
| | ethyl[4-(4-fluorophenyl)-3-[4-(methylsulfonyl)phenyl]-5-(trifluoromethyl)- |
| | 1H-pyrazol-1-yl]acetate; |
| D-156 | 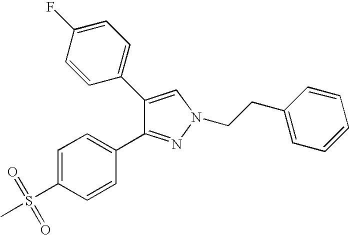 |
| | 4-(4-fluorophenyl)-3-[4-(methylsulfonyl)phenyl]-1-(2-phenylethyl)- |
| | 1H-pyrazole; |
| D-157 | 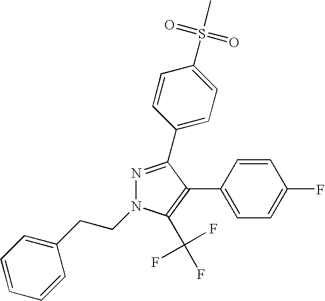 |
| | 4-(4-fluorophenyl)-3-[4-(methylsulfonyl)phenyl]-1-(2-phenylethyl)- |
| | 5-(trifluoromethyl)pyrazole; |
| D-158 | 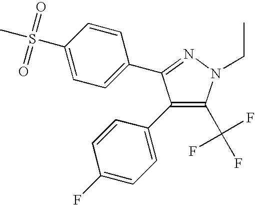 |
| | 1-ethyl-4-(4-fluorophenyl)-3-[4-(methylsulfonyl)phenyl]-5-(trifluoromethyl)- |
| | 1H-pyrazole; |
| D-159 | 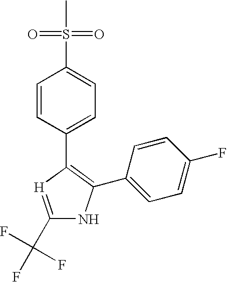 |
| | 5-(4-fluorophenyl)-4-(4-methylsulfonylphenyl)-2-trifluoromethyl- |
| | 1H-imidazole; |
| D-160 | 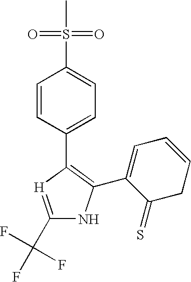 |
| | 4-[4-(methylsulfonyl)phenyl]-5-(2-thiophenyl)-2-(trifluoromethyl)- |
| | 1H-imidazole; |
| D-161 | 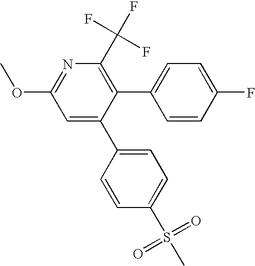 |
| | 5-(4-fluorophenyl)-2-methoxy-4-[4-(methylsulfonyl)phenyl)-6- |
| | (trifluoromethyl)pyridine; |
| D-162 | 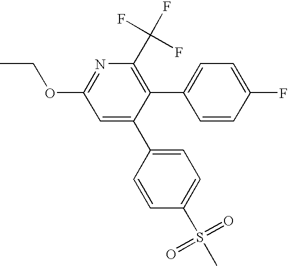 |
| | 2-ethoxy-5-(4-fluorophenyl)-4-[4-(methylsulfonyl)phenyl]-6- |
| | (trifluoromethyl)pyridine; |
| D-163 | 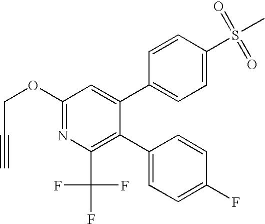 |
| | 5-(4-fluorophenyl)-4-[4-(methylsulfonyl)phenyl]-2-(2-propynyloxy)-6- |
| | (trifluoromethyl)pyridine; |
| D-164 | 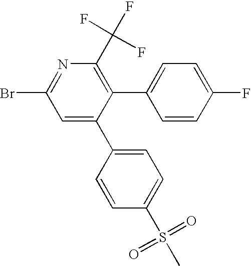 |
| | 2-bromo-5-(4-fluorophenyl)-4-[4-(methylsulfonyl)phenyl]- |
| | 6-(trifluoromethyl)pyridine; |
| D-165 | 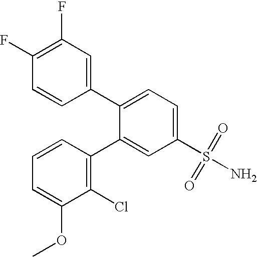 |
| | 4-[2-(3-chloro-4-methoxyphenyl)-4,5-difluorophenyl]benzenesulfonamide; |
| D-166 | 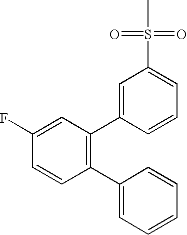 |
| | 1-(4-fluorophenyl)-2-[4-(methylsulfonyl)phenyl]benzene; |
| D-167 | 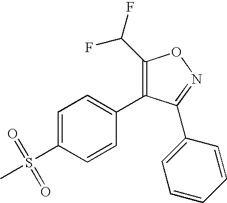 |
| | 5-difluoromethyl-4-(4-methylsulfonylphenyl)-3-phenylisoxazole; |
| D-168 | 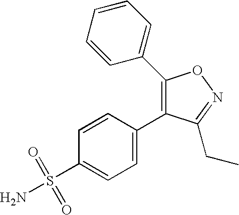 |
| | 4-[3-ethyl-5-phenylisoxazol-4-yl]benzenesulfonamide; |
| D-169 | 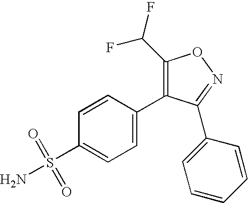 |
| | 4-[5-difluoromethyl-3-phenylisoxazol-4-yl]benzenesulfonamide; |
| D-170 | 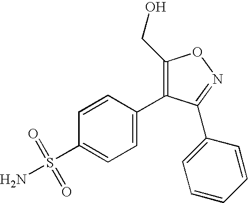 |
| | 4-[5-hydroxymethyl-3-phenylisoxazol-4-yl]benzenesulfonamide; |
| D-171 | 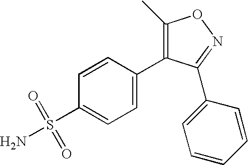 |
| | 4-[5-methyl-3-phenyl-isoxazol-4-yl]benzenesulfonamide; |
| D-172 | 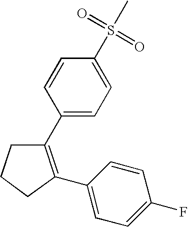 |
| | 1-[2-(4-fluorophenyl)cyclopenten-1-yl]-4-(methylsulfonyl)benzene; |
| D-173 | 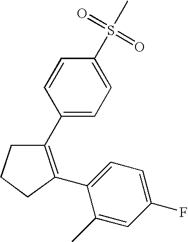 |
| | 1-[2-(4-fluoro-2-methylphenyl)cyclopenten-1-yl]-4-(methylsulfonyl)benzene; |
| D-174 | 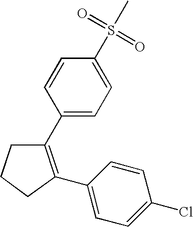 |
| | 1-[2-(4-chlorophenyl)cyclopenten-1-yl]-4-(methylsulfonyl)benzene; |
| D-175 | 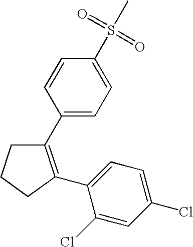 |
| | 1-[2-(2,4-dichlorophenyl)cyclopenten-1-yl]-4-(methylsulfonyl)benzene; |
| D-176 | 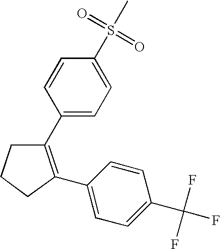 |
| | 1-[2-(4-trifluoromethylphenyl)cyclopenten-1-yl]4-(methylsulfonyl)benzene; |
| D-177 | 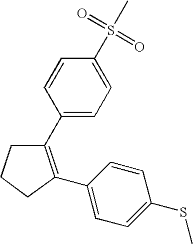 |
| | 1-[2-(4-methylthiophenyl)cyclopenten-1-yl]-4-(methylsulfonyl)benzene; |
| D-178 | 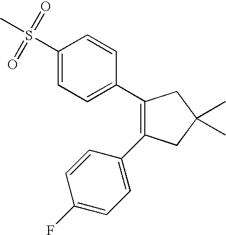 |
| | 1-[2-(4-fluoropheny1)4,4-dimethylcyclopenten-1-yl]- |
| | 4-(methylsulfonyl)benzene; |
| D-179 | 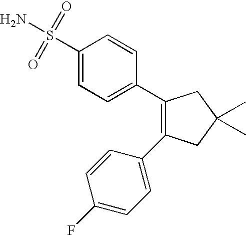 |
| | 4-[2-(4-fluorophenyl)-4,4-dimethylcyclopenten-1-yl]benzenesulfonamide; |
| D-180 | 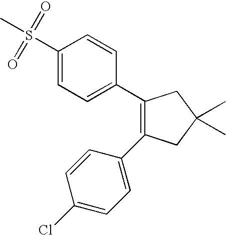 |
| | 1-[2-(4-chlorophenyl)-4,4-dimethylyclopenten-1-yl]- |
| | 4-(methylsulfonyl)benzene; |
| D-181 | 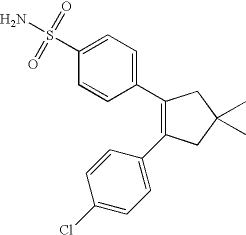 |
| | 4-[2-(4-chlorophenyl)-4,4-dimethylcyclopenten-1-yl]benzenesulfonamide; |
| D-182 | 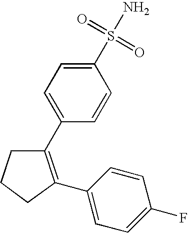 |
| | 4-[2-(4-fluorophenyl)cyclopenten-1-yl]benzenesulfonamide; |
| D-183 | 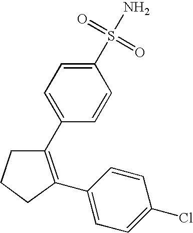 |
| | 4-[2-(4-chlorophenyl)cyclopenten-1-yl]benzenesulfonamide; |
| D-184 | 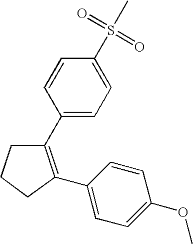 |
| | 1-[2-(4-methoxyphenyl)cyclopenten-1-yl]-4-(methylsulfonyl)benzene; |
| D-185 | 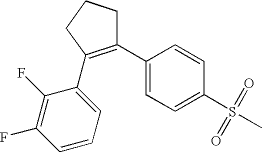 |
| | 1-[2-(2,3-difluorophenyl)cyclopenten-1-yl]-4-(methylsulfonyl)benzene; |
| D-186 | 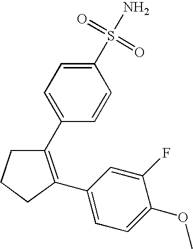 |
| | 4-[2-(3-fluoro-4-methoxyphenyl)cyclopenten-1-yl]benzenesulfonamide; |
| D-187 | 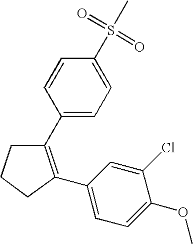 |
| | 1-[2-(3-chloro-4-methoxyphenyl)cyclopenten-1-yl]- |
| | 4-(methylsulfonyl)benzene; |
| D-188 | 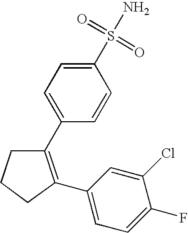 |
| | 4-[2-(3-chloro-4-fluorophenyl)cyclopenten-1-yl]benzenesulfonamide; |
| D-189 | 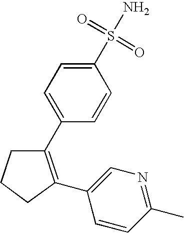 |
| | 4-[2-(2-methylpyridin-5-yl)cyclopenten-1-yl]benzenesulfonamide; |
| D-190 | 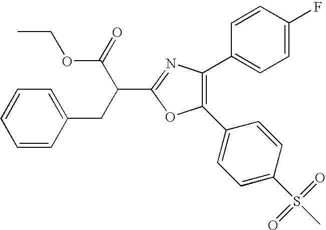 |
| | ethyl 2-[4-(4-fluorophenyl)-5-[4-(methylsulfonyl)phenyl]oxazol-2- |
| | yl]-2-benzyl-acetate; |
| D-191 | 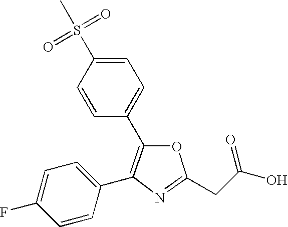 |
| | 2-[4-(4-fluorophenyl)-5-[4-(methylsulfonyl)phenyl]oxazol-2-yl]acetic acid; |
| D-192 | 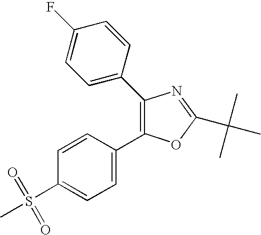 |
| | 2-(tert-butyl)-4-(4-fluorophenyl)-5-[4-(methylsulfonyl)phenyloxazole; |
| D-193 | 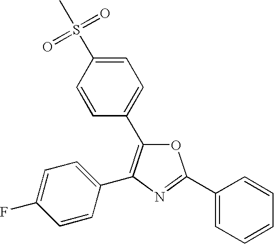 |
| | 4-(4-fluorophenyl)-5-[4-(methylsulfonyl)phenyl]-2-phenyloxazole; |
| D-194 | 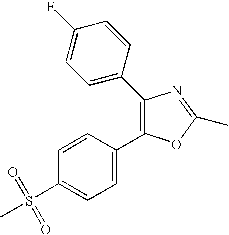 |
| | 4-(4-fluorophenyl)-2-methyl-5-[4-(methylsulfonyl)phenyl]oxazole; |
| D-195 | 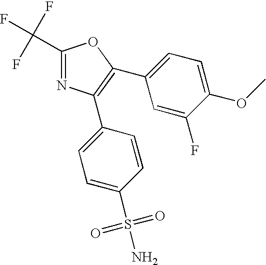 |
| | 4-[5-(3-fluoro-4-methoxyphenyl)-2-trifluoromethyl-4- |
| | oxazolyl]benzenesulfonamide; |
| D-196 | 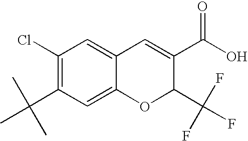 |
| | 6-chloro-7-(1,1-dimethylethyl)-2-trifluoromethyl-2H-1-benzopyran- |
| | 3-carboxylic acid; |
| D-197 | 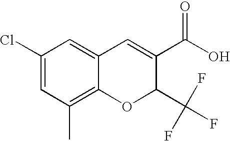 |
| | 6-chloro-8-methyl-2-trifluoromethyl-2H-1-benzopyran-3-carboxylic acid; |
| D-198 | 5,5-dimethyl-3-(3-fluorophenyl)-4-(4-methyl-sulphonyl-2(5H)-fluranone; |
| D-199 | 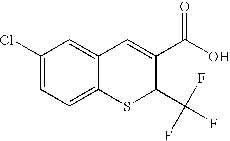 |
| | 6-chloro-2-trifluoromethyl-2H-1-benzothiopyran-3-carboxylic acid; |
| D-200 | 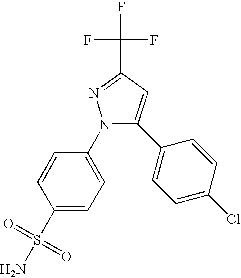 |
| | 4-[5-(4-chlorophenyl)-3-(trifluoromethyl)-1H-yl]benzenesulfonamide; |
| D-201 | 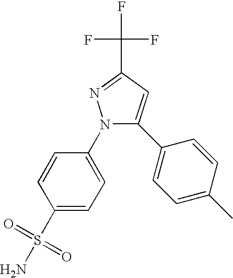 |
| | 4-[5-(4-methylphenyl)-3-(trifluoromethyl)-1H-pyrazol-1- |
| | yl]benzenesulfonamide; |
| D-202 | 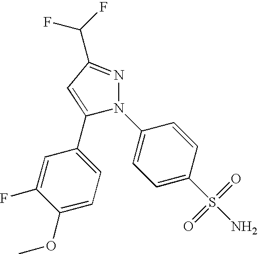 |
| | 4-[5-(3-fluoro-4-methoxyphenyl)-3-(difluoromethyl)-1H- |
| | pyrazol]benzenesulfonamide; |
| D-203 | 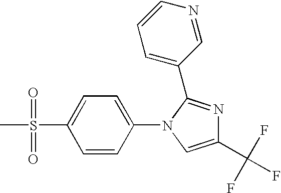 |
| | 3-[1-[4-(methylsulfonyl)phenyl]-4-trifluoromethyl-1H-imidazol-2- |
| | yl]pyridine; |
| D-204 | 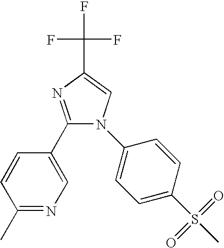 |
| | 2-methyl-5-[1-[4-(methylsulfonyl)phenyl]-4-trifluoromethyl-1H- |
| | imidazol-2-yl]pyridine; |
| D-205 | 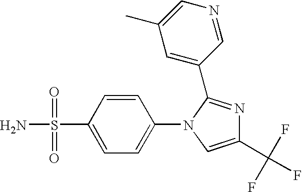 |
| | 4-[2-(5-methylpyridin-3-yl)-4-(trifluoromethyl)-1H-imidazol-1- |
| | yl]benzenesulfonamide; |
| D-206 | 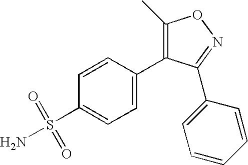 |
| | 4-[5-methyl-3-phenylisoxazol-4-yl]benzenesulfonamide; |
| D-207 | 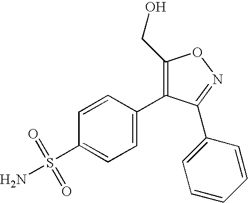 |
| | 4-[5-hydroxymethyl-3-phenylisoxazol-4-yl]benzenesulfonamide; |
| D-208 | 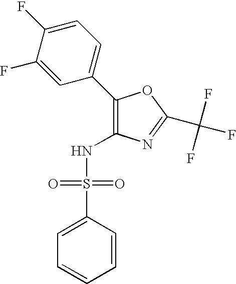 |
| | [2-trifluoromethyl-5-(3,4-difluorophenyl)-4-oxazolyl]benzenesulfonamide; |
| D-209 | 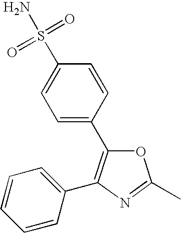 |
| | 4-[2-methyl-4-phenyl-5-oxazolyl]benzenesulfonamide; |
| D-210 | 4-[5-(3-fluoro-4-methoxyphenyl-2-trifluoromethyl)-4- |
| | oxazolyl]benzenesulfonamide; |
| D-211 | 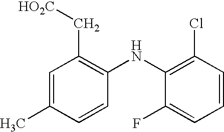 |
| | [2-(2-Chloro-6-fluoro-phenylamino)-5-methyl-phenyl]-acetic acid or |
| | COX 189 or Lumiracoxib |
| D-212 | 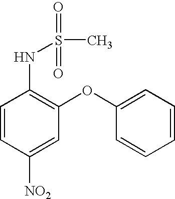 |
| | N-(4-Nitro-2-phenoxy-phenyl)-methanesulfonamide or Nimesulide |
| D-213 | 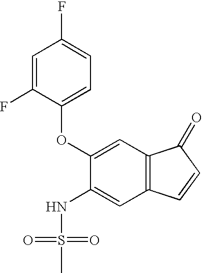 |
| | N-[6-(2,4-Difluoro-phenoxy)-1-oxo-inden-5-yl]-methanesulfonamide or |
| | Flosulide |
| D-214 | 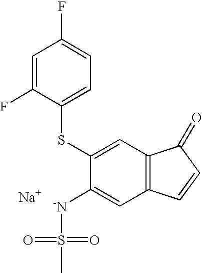 |
| | N-[6-(2,4-Difluoro-phenylsulfanyl)-1-oxo-1H-inden-5- |
| | yl]-methanesulfonamide, soldium salt, or L-745337 |
| D-215 | 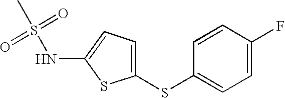 |
| | N-[5-(4-fluorophenylsulfanyl)-thiophen-2- |
| | yl]-methanesulfonaniide or RWJ-63556 |
| D-216 | L-784512 |
| D-217 | 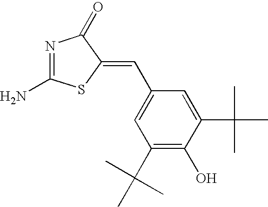 |
| | (5Z)-2-amino-5-[[3,5-bis(1,1-dimethylethyl)-4-hydroxyphenyl]methylene]- |
| | 4(5H)-thiazolone or Darbufelone |
| D-218 | CS-502 |
| D-219 | LAS-34475 |
| D-220 | LAS-34555 |
| D-221 | S-33516 |
| D-222 | SD-8381 |
| D-223 | L-783003; |
| D-224 | 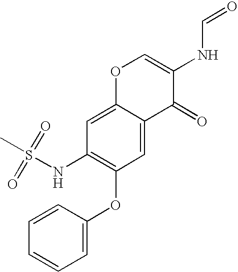 |
| | N-[3-(formylamino)-4-oxa-6-phenoxy-4H-1-benzopyran-7- |
| | yl]-methanesulfonamide or T614 |
| D-225 | D-1367 |
| D-226 | L-748731 |
| D-227 | 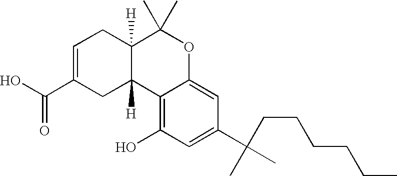 |
| | (6aR,10aR)-3-(1,1-dimethylheptyl)-6a,7,10,10a-tetrahydro-1-hydroxy-6,6- |
| | dimethyl-6H-dibenzo[b,d]pyran-9-carboxylic acid or CT 3 |
| | CT3 |
| D-228 | CGP-28238 |
| D-229 | 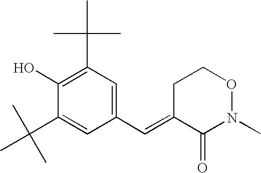 |
| | 4-[[3,5-bis(1,1-dimethylethyl)-4-hydroxyphenyl]methylene]dihydro-2- |
| | methyl-2H-1,2-oxazin-3(4H)-one or BF-389 |
| D-230 | GR-253035 |
| D-231 | 6-dioxo-9H-purin-8-yl-cinnamic acid |
| D-232 | S-2474 |

Further, according to another embodiment of the present invention, in combination with an ASBT inhibitor of Table 2, the COX-2 selective inhibitors noted above (Table 7A) may be selected from D-1, D-2, D-3, D-4, D-5, D-6, D-7, D-8, D-9, D-10, D-11, D-12, D-13, D-14, D-15, D-16, D-17, celecoxib (D-18), D-19, D-20, rofecoxib (D-21), D-22, D-23, D-24, D-25, D-26, D-27, D-28, D-29, D-30, D-31, D-32, D-33, D-34, D-35, D-36, D-37, D-38, D-39, D-40, D-41, D-42, D-43, D-44, D-45, D-46, D-47, D-48, D-49, D-50, D-51, D-52, D-53, D-54, D-55, D-56, D-57, D-58, D-59, D-60, D-61, D-62, D-63, D-64, D-65, D-66, D-67, D-68, D-69, D-70, D-71, D-72, D-73, D-74, D-75, D-76, D-77, D-78, D-79, D-80, D-81, D-82, D-83, D-84, D-85, D-86, D-87, D-88, D-89, D-90, D-91, D-92, D-93, D-94, D-95, D-96, D-97, D-98, D-99, D-100, D-101, D-102, D-103, D-104, D-105, D-106, D-107, D-108, D-109, D-110, D-111, D-112, D-113, D-114, D-115, D-116, D-117, D-118, D-119, D-120, D-121, D-122, D-123, D-124, D-125, D-126, D-127, D-128, D-129, D-130, D-131, D-132, D-133, D-134, D-135, D-136, D-137, D-138, D-139, D-140, D-141, D-142, D-143, D-144, D-145, D-146, D-147, D-148, D-149, D-150, D-151, D-152, D-153, D-154, D-155, D-156, D-157, D-158, D-159, D-160, D-161, D-162, D-163, D-164, D-165, D-166, D-167, D-168, D-169, D-170, D-171, D-172, D-173, D-174, D-175, D-176, D-177, D-178, D-179, D-180, D-181, D-182, D-183, D-184, D-185, D-186, D-187, D-188, D-189, D-190, D-191, D-192, D-193, D-194, D-195, D-196, D-197, D-198, D-199, D-200, D-201, D-202, D-203, D-204, D-205, D-206, D-207, D-208, D-209, D-210, D-211, D-212, D-213, D-214, D-215, D-216, D-217, D-218, D-219, D-220, D-221, D-222, D-223, D-224, D-225, D-226, D-227, D-228, D-229, D-230, D-231, D-232, or an isomer, a pharmaceutically acceptable salt, ester, or prodrug thereof. Even further, according to another embodiment of the present invention, in combination with the ASBT inhibitors of Table 2, the COX-2 selective inhibitors noted above (Table 7A) may be selected from D-1 to D-5, D-6 to D-10, D-11 to D-15, D-16 to D-20, D-21 to D-25, D-26 to D-30, D-31 to D-35, D-36-D-40, D-41 to D-45, D-46 to D-50, D-51 to D-55, D-56 to D-60, D-61 to D-65, D-66 to D-70, D-71 to D-75, D-76 to D-80, D-81 to D-85, D-D-86 to D-90, D-91 to D-95, D-96 to D-100, D-101 to D-105, D-106 to D-1l0, D-111 to D-115, D-116 to D-120, D-121 to D-125, D-126 to D-130, D-131 to D-135, D-136 to D-140, D-141 to D-145, D-146 to D-150, D-151 to D-155, D-156 to D-160, D-161 to D-165, D-166 to D-170, D-171 to D-175, D-176 to D-180, D-181 to D-185, D-186 to D-190, D-191 to D-195, D-196 to D-200, D-201 to D-205, D-206 to D-210, D-211 to D-215, D-216 to D-220, D-221 to D-225, D-226 to D-230, D-231-D-232 or combinations thereof.

e. HENG-CoA Reductase Inhibitors

The present invention discloses that treatment of a subject with one or more ASBT inhibitors, one or more cyclooxygenase-2 selective inhibitors and one or more HMG-CoA reductase inhibitors results in the prophylaxis and/or treatment of cardiovascular conditions and/or disorders relative to other combination regimens. The method comprises treating the subject with an amount of an ASBT inhibitor, an amount of a cyclooxygenase-2 selective inhibitor or its prodrug and an amount of an HMG-CoA inhibitor, wherein the amount of the ASBT inhibitor, the amount of the cyclooxygenase-2 selective inhibitor and the amount of the HMG-CoA inhibitor together constitute a hypercholesterolemia-related condition effective amount or an inflammation-related condition effective amount of the said compounds.

For example, one of the many embodiments of the present invention is a combination therapy comprising therapeutic dosages of an ASBT inhibitor described above, therapeutic dosages of a cyclooxygenase-2 selective inhibitor described above and therapeutic dosages of an HMG-CoA reductase inhibitor as herein provided.

HMG Co-A reductase inhibitors encompassing a wide range of structures are useful in the methods and combinations of the present invention. Such HMG Co-A reductase inhibitors may be, for example, statins that have been synthetically or semi-synthetically prepared, statins extracted from natural sources such as plants, or statins isolated as fungal metabolites from cultures of suitable microorganisms. Nonlimiting examples of HMG Co-A reductase inhibitors that may be used in the present invention include those HMG Co-A reductase inhibitors disclosed by way of example and not limitation in Table 8, including the diastereomers, enantiomers, racemates, salts, tautomers, conjugate acids, and prodrugs thereof. The therapeutic compounds of Table 8 can be used in the present, invention in a variety of forms, including acid form, salt form, racemates, enantiomers, zwitterions, and tautomers.

TABLE 8
Examples of HMG-CoA Reductase Inhibitors as
Embodiments

| | CAS Numbers for | |
|---|---|---|
| | Specific and | |
| Compounds and | Representative | |
| Compound Classes | Compounds | Reference |
| Benfluorex | 23602-78-0 | ES 474498, Servier |
| Fluvastatin | 93957-54-1 | EP 244364, Sandoz |
| Lovastatin | 75330-75-5 | EP 22478, Merck & Co. |
| Pravastatin | 81093-37-0 | DE 3122499, Sankyo |
| Simvastatin | 79902-63-9 | EP 33538, Merck & Co. |
| Atorvastatin | 134523-00-5 | EP 409281, Warner- |
| | | Lambert |
| Cerivastatin | 145599-86-6 | JP 08073-432, Bayer |
| Bervastatin | 132017-01-7 | EP 380392, Merck KGaA |
| Rosuvastatin | 147098-20-2 | U.S. Pat. No. 5,260,440, Shionogi |
| (ZD-4522) | | |
| Itavastatin | 141750-63-2 | WO 97/23200, Kowa |
| Dalvastatin | 132100-55-1 | Kuttar et al., J. |
| | | Chromatogr., A 678, |
| | | 259-63 (1994); Rhone- |
| | | Poulenc Rorer |
| Mevastatin | 73573-88-3 | JP 56051992; Sankyo |
| ZD 9720 | | WO 97/06802 |
| ZD 4522 | 147098-20-2 | EP 521471; Bioorg. |
| | (calcium salt); | Med. Chem., 5, 437-444 |
| | 147098-18-8 | (1997); Drugs Future, |
| | (sodium salt) | 24, 511-513 (1999) |
| BMS 180431 | 129829-03-4 | Sit et al., J. Med. |
| | | Chem., 33, 2982-99 |
| | | (1990); Bristol-Myers |
| | | Squibb |
| NK 104 | 141750-63-2 | Takano et al., |
| | | Tetahedron: Assymetry, |
| | | 4, 201-4 (1993); |
| | | Nissan Chemical |
| (Carboxydihydroxy- | 148966-78-3, 139993- | EP 464845; Shionogi |
| heptenyl)- | 44-5, 139993- | |
| sulfonylpyrroles, | 45-6, 139993- | |
| including S 4522 | 46-7, 139993-47- | |
| | 8, 139993-48-9, | |
| | 139 993-49-0, | |
| | 139993-50-3, 139993- | |
| | 51-4, 139993- | |
| | 52-5, 139993-53- | |
| | 6, 139 993-54- | |
| | 7, 139993-55-8, | |
| | 139993-56-9, 139993- | |
| | 57-0, 139993- | |
| | 58-1, 139993- | |
| | 59-2, 139993-60- | |
| | 5, 139993-61-6, | |
| | 139993-62-7, 139993- | |
| | 63-8, 139993- | |
| | 64-9, 139993- | |
| | 65-0, 139993- | |
| | 66-1, 139993-67- | |
| | 2, 139993-68-3, | |
| | 139993-69-4, 139993- | |
| | 70-7, 139993- | |
| | 71-8, 139993-72- | |
| | 9, 139993-73-0, | |
| | 139 993-74-1, | |
| | 139993-75-2, | |
| | 139993-76-3, | |
| | 139993-77-4, 139993- | |
| | 78-5, 139993- | |
| | 79-6, 139993-80- | |
| | 9, 140110-63-0, | |
| | 140128-98-9, 140128- | |
| | 99-0, 140157- | |
| | 62-6 | |
| Boron analogs of di- | 125894-01-1, 125894- | Sood et al., Eur. J. |
| and tripeptides | 02-2, 125894- | Med. Chem., 25, 301-8 |
| | 03-3, 125894-04- | (1990); Boron |
| | 4, 125894-05-5, | Biologicals |
| | 125894-08-8, 125894- | |
| | 09-9, 125914- | |
| | 96-7 | |
| Zaragozic Acids | 157058-13-4, 157058- | GB 2270312 |
| | 14-5, 157058- | |
| | 15-6, 157058-16- | |
| | 7, 157058-17-8, | |
| | 157058-18-9, 157058- | |
| | 19-0 | |
| Seco-oxysterol | 157555-28-7, | Larsen et al., J. Med. |
| analogs, including U | 157-555-29-8 | Chem., 37, 2343-51 |
| 88156 | | (1994); Pharmacia & |
| | | Upjohn |
| Pyridopyrimidines, | 64405-40-9, | Hermecz et al., Hung. |
| including acitemate | 101197-99-3 | Arzneim-Forsch., 29, |
| | | 1833-5 (1979); |
| | | Mitsubishi |
| BMS 22566 | 129829-03-4 | Sit et al., J. Med. |
| | | Chem., 33, 2982-99 |
| | | (1990); Bristol- |
| | | Meyers-Squibb |
| Colestolone | 50673-97-7 | Raulston et al., |
| | | Biochem. Biophys. Res. |
| | | Commun., 71, 984-9 |
| | | (1976); American Home |
| | | Products |
| CP 83101 | 130746-82-6, | Wint and McCarthy, J. |
| | 130778-27-7 | Labelled Compd. |
| | | Radiopharm., 25, 1289-97 |
| | | (1988); Pfizer |
| Dihydromevinolin | 77517-29-4 | Falck and Yang, |
| | | Tetrahedron Lett., 25, |
| | | 3563-66 (1984); Merck |
| | | & Co. |
| DMP 565 | | Ko et al., Abstr. |
| | | Papers Am. Chem. Soc. |
| | | (207^{th} Nat. Meeting, |
| | | Part 1, MEDI 10, |
| | | (1994); Dupont Merck |
| Pyridyl and | 122254-45-9 | Beck et al., J. Med. |
| Pyrimidinylethenyl- | | Chem., 33, 52-60 |
| desmethylmevalonates | | (1990); Hoechst Marion |
| including glenvastin | | Roussel |
| GR 95030 | 157243-22-6 | U.S. Pat. No. 5,316,765; Glaxo |
| | | Wellcome |
| Isoxazolopyridyl- | 130581-42-9, 130581- | EP 369323 |
| mevalonates, | 43-0, 130581- | |
| carboxylic acids and | 44-1, 130581- | |
| esters | 45-2, 130581- | |
| | 46-3, 130581- | |
| | 47-4, 130581- | |
| | 48-5, 130581- | |
| | 49-6, 130581- | |
| | 50-9, 130581- | |
| | 51-0, 13081- | |
| | 52-1, 130619- | |
| | 07-7, 130619-08- | |
| | 8, 130619-09-9 | |
| Lactones of 6- | 127502-48-1, | Jenderella et al., J. |
| phenoxy-3,5- | 13606-66-1, | Med. Chem., 34, 2962-83 |
| dihydroxy-hexanoic | 136034-04-3 | (1991); Hoechst |
| acids | | Marion Roussel |
| L 659699 | 29066-42-0 | Chiang et al., J. Org. |
| | | Chem., 54, 5708-12 |
| | | (1989); Merck & Co. |
| L 669262 | 130468-11-0 | Stokker, J. Org. |
| | | Chem., 59, 5983-6 |
| | | (1994); Merck & Co. |
| Pannorin | 137023-81-5 | Ogawa et al., J. |
| | | Antibiot., 44, 762-7 |
| | | (1991); Toyoko Noko |
| | | Univ |
| Rawsonol | 125111-69-5 | Cane et al., |
| | | Phytochemistry, 28, |
| | | 2917-19 (1989); |
| | | SmithKline Beecham |
| RP 61969 | 126059-69-6 | EP 326386; Phone- |
| | | Poulenc Rorer |
| Bile acid-derived | | Kramer et al., |
| HMG Co-A reductase | | Biochim. Biophys. |
| inhibitors; Na S | | Acta, 1227, 137-54 |
| 2467 and S 2468 | | (1994); Hoechst Marion |
| | | Roussel |
| SC 32561 | 76752-41-5 | U.S. Pat. No. 4,230,626; Monsanto |
| SC 45355 | 125793-76-2 | EP 329124; non- |
| | | industrial source |
| Phosphorus- | 133983-25-2 | U.S. Pat. No. 5,274,155; Bristol- |
| containing HMG Co-A | | Myers Squibb |
| reductase inhibitors | | |
| including SQ 33600 | | |
| 6-Aryloxymethyl-4- | 135054-71-6, 136215- | EP 418648 |
| hydroxytetrahydro- | 82-2, 136215- | |
| pyran-2-ones, car- | 83-3, 136215- | |
| boxylic acids and | 84-4, 136215- | |
| salts | 85-5, 136315-18- | |
| | 9, 136315-19-0, | |
| | 136315-20-3, 136315- | |
| | 21-4, 136316- | |
| | 20-6 | |
| Atorvastatin calcium | 134523-03-8 | Baumann et al., |
| (CI 981) | | Tetrahedron Lett., 33, |
| | | 2283-4 (1992). |
| Mevinolin analogs | | EP 245003 |
| Pyranone derivatives | | U.S. Pat. No. 4,937,259 |
| 1,2,4-Triazolidine- | 16044-43-2 | WO 9000897 |
| 3,5-diones | | |
| Isoazolidine-3,5- | 124756-24-7 | EP 321090 |
| diones | | |
| CS 514 | 81181-70-6 | DE 3122499 |
| 1,10-Bis(carboxy- | 32827-49-9 | DE 2038835 |
| methylthio)decane | | |
| α, β-, and γ- | | Huang and Hall, Eur. |
| Alkylaminophenone | | J. Med. Chem., 31, |
| analogs, including | | 281-90 (1996) |
| N-phenyl-piperazino- | | |
| propiophenone | | |
| 3-Amino-1-(2,3,4- | | Huang and Hall, Arch. |
| mononitro-, mono- or | | Pharm., 329, 339-346 |
| dihalophenyl)propan- | | (1996) |
| 1-ones, including 3- | | |
| morpholino- or | | |
| piperidino-1-(3- | | |
| nitrophenyl)-propan- | | |
| 1-ones | | |
| Substituted | 64769-68-2 | U.S. Pat. No. 4,049,813 |
| isoxazolo | | |
| pyridinones | | |
| Biphenyl derivatives | | JP 07089898 |
| 4-[1-(Substituted | | Watanabe et al., Eur. |
| phenyl)-2-oxopyr- | | J. Med. Chem., 29, |
| rolidin-4-yl]meth- | | 675-86 (1994) |
| oxybenzoic acids | | |
| Dihydroxy(tetra- | | U.S. Pat. No. 5,134,155 |
| hydro-indazolyl, | | |
| tetrahydrocyclo- | | |
| pentapyrazolyl, or | | |
| hexahydrocyclohepta- | | |
| pyrazole)heptenoate | | |
| derivatives | | |
| A 1233 | | Kitasato University |
| BAY-w-9533 | | Bayer |
| BB 476 | | British Biotech |
| BMS 180436 | | Bristol-Myers Squibb |
| Chiral HMG Co-A | | Chiroscience |
| reductase inhibitors | | |
| Isoxazolopyridine | | Nissan Chemical |
| HMG Co-A reductase | | |
| inhibitors | | |
| Seco-oxysterol HMG | | Pharmacia & Upjohn |
| Co-A reductase | | |
| inhibitors | | |
| Thiophene HMG Co-A | | Sandoz |
| reductase inhibitors | | |
| HMG Co-A reductase | | Hoechest Marion |
| inhibitors, 6-phenoxy- | | Roussel |
| 3,5-dihydroxy- | | |
| hexanoic acids | | |
| N-((1-Methylpropyl)- | | Sandoz |
| carbonyl)-8-(2- | | |
| (tetrahydro-4-hydroxy- | | |
| 6-oxo-2H-pyran- | | |
| 2-yl)ethyl)-per- | | |
| hydroisoquinoline | | |
| N-(1-Oxododecyl)- | | Hoechst Marion Roussel |
| 4α,10-dimethyl-8- | | |
| aza-trans-deca-3γ-ol | | |
| P 882222 | | Nissan Chemical |
| S 853758A | | Hoechst Marion Roussel |
| (S)-4-((2-(4-(4-Fluorophenyl)- | | Bristol-Myers Squibb |
| 5-methyl- | | |
| 2-(1-methylethyl)-6- | | |
| phenyl-3-pyridinyl)- | | |
| ethenyl)hydroxyphosphinyl)- | | |
| 3-hydroxy- | | |
| butanoic acid, | | |
| disodium salt | | |
| SDZ 265859 | | Sandoz |
| (4R-(4α,6β (E)))-6- | | Warner Lambert |
| (2-(5-(4-Fluoro- | | |
| phenyl)-3-(1-methyl- | | |
| ethyl)-1-(2- | | |
| pyridinylpyrazol-4- | | |
| yl)ethenyl)tetrahydro- | | |
| 4-hydroxy-2H- | | |
| pyran-2-one | | |
| 5β-aminoethyl- | | Boehringer Mannheim |
| thiopentanoic acid | | |
| derivatives | | |
| 6-Amino-2-mercapto- | | North Carolina |
| 5-methylpyrimidine- | | University |
| 4-carboxylic acid | | |
| 6-Phenoxymethyl-and | | Hoechst Marion Roussel |
| 6-phenylethylen-(4- | | |
| hydroxy- | | |
| tetrahydropyran-2- | | |
| one)analogues | | |

In a preferred embodiment of the present invention the HMG-CoA reductase inhibitors are described in Table 9 below. The individual patent documents referenced in Table 9 describe the prepraration of these statins and are each herein incorporated by reference.

In an even more preferred embodiment of the invention the HMG-CoA inhibitor is selected from the group of statins consisting of atorvastatin, simvastatin, pravastatin, lovastatin, rosuvastatin and itavastatin.

TABLE 9
References for Preparation of HMG-CoA Reductase
Inhibitors

| | | | Patent/Literature |
|---|---|---|---|
| | | CAS | Reference for |
| Compound | | Registry | Preparation of Compound |
| Number | Common Name | Number | Per Se |
| C-1 | Fluvastatin | 93957-54-1 | U.S. Pat. No. 4,739,073; |
| | | | U.S. Pat. No. 5,354,772 |
| C-2 | Lovastatin | 75330-75-5 | U.S. Pat. No. 4,231,938 |
| C-3 | Pravastatin | 81093-37-0 | U.S. Pat. No. 4,346,227 |
| C-4 | Simvastatin | 79902-63-9 | U.S. Pat. No. 4,444,784 |
| C-5 | Atorvastatin | 134523-00-5 | EP 409281; |
| | | | U.S. Pat. No. 5,273,995 |
| C-6 | Cerivastatin | 145599-86-6 | U.S. Pat. No. 5,177,080 |
| C-7 | Bervastatin | 132017-01-7 | EP 380392 |
| C-8 | Rosuvastatin | 147098-20-2 | U.S. Pat. No. 5,260,440 |
| C-9 | Itavastatin | 141750-63-2 | WO 97/23200, Kowa |

Another embodiment of the present invention comprises a therapeutic combination containing an amount of an apical sodium co-dependent bile acid transport inhibitor, an amount of a cyclooxygenase-2 selective inhibitor or its prodrug and an amount of an HMG-CoA reductase inhibitor, and a pharmaceutically acceptable carrier, wherein the amount of the apical sodium co-dependent bile acid transport inhibitor, the amount of the cyclooxygenase-2 selective inhibitor and the amount of the HMG-CoA inhibitor together constitute a hypercholesterolemia-related condition effective amount or an inflammation-related condition effective amount of the said compounds. For example, one of the many embodiments of the present invention is a combination comprising therapeutic dosages of an ASBT inhibitor selected from Table 2, a cyclooxygenase-2 selective inhibitor selected from Tables 4, 6 and 7A and an HMG-CoA inhibitor selected from Table 8 or Table 9. A preferred embodiment of the present invention is a combination comprising therapeutic dosages of a benzothiepine ASBT inhibitor, a tricyclic cyclooxygenase-2 selective inhibitor and a statin HMG-CoA inhibitor.

f. Dosages, Formulations, and Routes of Administration

Many of the compounds useful in the present invention can have at least two asymmetric carbon atoms, and therefore include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention. Isomers may include geometric isomers, for example cis-isomers or trans-isomers across a double bond. All such isomers are contemplated among the compounds useful in the present invention. The compounds useful in the present invention also include tautomers.

The compounds useful in the present invention as discussed below include their salts, solvates and prodrugs.

The combinations of the present invention can be administered for the prophylaxis and treatment of hyperlipidemic and cardiovascular diseases or conditions by any means, preferably oral, that produce contact of these compounds with their site of action in the body, for example in the ileum of a mammal, e.g., a human.

For the prophylaxis or treatment of the conditions referred to above, the compounds useful in the combinations and methods of the present invention can be used as the compound per se. Pharmaceutically acceptable salts are particularly suitable for medical applications because of their greater aqueous solubility relative to the parent compound. Such salts must clearly have a pharmaceutically acceptable anion or cation. Suitable pharmaceutically acceptable acid addition salts of the compounds of the present invention when possible include those derived from inorganic acids, such as hydrochloric, hydrobromic, phosphoric, metaphosphoric, nitric, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, succinic, toluenesulfonic, tartaric, and trifluoroacetic acids. The chloride salt is particularly preferred for medical purposes. Suitable pharmaceutically acceptable base salts include ammonium salts, alkali metal salts such as sodium and potassium salts, and alkaline earth salts such as magnesium and calcium salts.

The anions useful in the present invention are, of course, also required to be pharmaceutically acceptable and are also selected from the above list.

The compounds useful in the present invention can be presented with an acceptable carrier in the form of a pharmaceutical combination. The carrier must, of course, be acceptable in the sense of being compatible with the other ingredients of the combination and must not be deleterious to the recipient. The carrier can be a solid or a liquid, or both, and is preferably formulated with the compound as a unit-dose combination, for example, a tablet, which can contain from 0.05% to 95% by weight of the active compound. Other pharmacologically active substances can also be present, including other compounds of the present invention. The pharmaceutical combinations of the invention can be prepared by any of the well known techniques of pharmacy, consisting essentially of admixing the components.

These compounds can be administered by any conventional means available for use in conjunction with pharmaceuticals, either as individual therapeutic compounds or as a combination of therapeutic compounds.

The amount of compound which is required to achieve the desired biological effect will, of course, depend on a number of factors such as the specific compound chosen, the use for which it is intended, the mode of administration, and the clinical condition of the recipient.

In general, a total daily dose of an ASBT inhibitor can be in the range of from about 0.01 to about 20 mg/day, preferably from about 0.1 to about 10 mg/day, more preferably from about 0.5 to about 5.0 mg/day.

A total daily dose of a cyclooxygenase-2 selective inhibitor can be in the range of from about 0.3 to about 100 mg/kg body weight/day, preferably from about 1 to about 50 mg/kg body weight/day, more preferably from about 3 to about 10 mg/kg body weight/day.

A total daily dose of an HMG-CoA reductase inhibitor can generally be in the range of from about 0.1 to about 100 mg/day in single or divided doses. Lovastatin, atorvastatin, or mevastatin, for example, generally are each administered separately in a daily dose of about 10 to about 80 mg/day. Fluvastatin is generally administered in a daily dose of about 20 to about 40 mg/day. Cerivastatin is generally administered in a daily dose of about 0.1 to about 0.3 mg/day.

The daily doses described in the preceding paragraphs for the various therapeutic compounds can be administered to the patient in a single dose, or in proportionate multiple subdoses. Subdoses can be administered 2 to 6 times per day. Doses can be in sustained release form effective to obtain desired results.

In the case of pharmaceutically acceptable salts, the weights indicated above refer to the weight of the acid equivalent or the base equivalent of the therapeutic compound derived from the salt.

Oral delivery of the combinations of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the present invention (e.g., ASBT inhibitors), the intended effect is to extend the time period over which the active drug molecule is delivered to the site of action (e.g., the ileum) by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

The combinations of the present invention can be delivered orally either in a solid, in a semi-solid, or in a liquid form. When in a liquid or in a semi-solid form, the combinations of the present invention can, for example, be in the form of a liquid, syrup, or contained in a gel capsule (e.g., a gel cap).

When administered intravenously, the dose for an ASBT inhibitor can, for example, be in the range of from about 0.01 mg to about 20 mg/day, preferably from about 0.1 to about 10 mg/day, more preferably from about 0.5 to about 5.0 mg/day.

For a cyclooxygenase-2 selective inhibitor the intravenously administered dose can, for example, be in the range of from about 0.003 to about 1.0 mg/kg body weight/day, preferably from about 0.01 to about 0.75 mg/kg body weight/day, more preferably from about 0.1 to about 0.6 mg/kg body weight/day.

An HMG-CoA reductase inhibitor can be intravenously administered, for example, in the range of from about 0.03 to about 5.0 mg/kg body weight/day, preferably from about 0.1 to about 1.0 mg/kg body weight/day, more preferably from about 0.4 to about 0.6 mg/kg body weight/day.

The dose of any of these therapeutic compounds can be conveniently administered as an infusion of from about 10 ng/kg body weight to about 100 ng/kg body weight per minute. Infusion fluids suitable for this purpose can contain, for example, from about 0.1 ng to about 10 mg, preferably from about 1 ng to about 10 mg per milliliter. Unit doses can contain, for example, from about 1 mg to about 10 g of the compound of the present invention. Thus, ampoules for injection can contain, for example, from about 1 mg to about 100 mg.

Pharmaceutical combinations according to the present invention include those suitable for oral, rectal, topical, buccal (e.g., sublingual), and parenteral (e.g., subcutaneous, intramuscular, intradermal, or intravenous) administration, although the most suitable route in any given case will depend on the nature and severity of the condition being treated and on the nature of the particular compound which is being used. In most cases, the preferred route of administration is oral.

Pharmaceutical combinations suitable for oral administration can be presented in discrete units, such as capsules, cachets, lozenges, or tablets, each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such combinations can be prepared by any suitable method of pharmacy which includes the step of bringing into association the active compound(s) and the carrier (which can constitute one or more accessory ingredients). In general, the combinations are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Pharmaceutical combinations suitable for buccal (sub-lingual) administration include lozenges comprising a compound of the present invention in a flavored base, usually sucrose, and acacia or tragacanth, and pastilles comprising the compound in an-inert base such as gelatin and glycerin or sucrose and acacia.

Pharmaceutical combinations suitable for parenteral administration conveniently comprise sterile aqueous preparations of a compound of the present invention. These preparations are preferably administered intravenously, although administration can also be effected by means of subcutaneous, intramuscular, or intradermal injection. Such preparations can conveniently be prepared by admixing the compound with water and rendering the resulting solution sterile and isotonic with the blood. Injectable combinations according to the invention will generally contain from 0.1 to 5% w/w of a compound disclosed herein.

Pharmaceutical combinations suitable for rectal administration are preferably presented as unit-dose suppositories. These can be prepared by admixing a compound of the present invention with one or more conventional solid carriers, for example, cocoa butter, and then shaping the resulting mixture.

Pharmaceutical combinations suitable for topical application to the skin preferably take the form of an ointment, cream, lotion, paste, gel, spray, aerosol, or oil. Carriers which can be used include petroleum jelly (e.g., Vaseline), lanolin, polyethylene glycols, alcohols, and combinations of two or more thereof. The active compound is generally present at a concentration of from 0.1 to 50% w/w of the combination, for example, from 0.5 to 2%.

Transdermal administration is also possible. Pharmaceutical combinations suitable for transdermal administration can be presented as discrete patches adapted to remain in intimate contact with the epidermis of the recipient for a prolonged period of time. Such patches suitably contain a compound of the present invention in an optionally buffered, aqueous solution, dissolved and/or dispersed in an adhesive, or dispersed in a polymer. A suitable concentration of the active compound is about 1% to 35%, preferably about 3% to 15%. As one particular possibility, the compound can be delivered from the patch by electrotransport or iontophoresis, for example, as described in Pharmaceutical Research, 3, 318 (1986).

In any case, the amount of active ingredient that can be combined with carrier materials to produce a single dosage form to be administered will vary depending upon the host treated and the particular mode of administration.

The solid dosage forms for oral administration including capsules, tablets, pills, powders, gel caps, and granules noted above comprise one or more compounds useful in the present invention admixed with at least one inert diluent such as sucrose, lactose, or starch. Such dosage forms may also comprise, as in normal practice, additional substances other than inert diluents, e.g., lubricating agents such as magnesium stearate or solubilizing agents such as cyclodextrins. In the case of capsules, tablets, powders, granules, gel caps, and pills, the dosage forms may also comprise buffering agents. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such combinations may also comprise adjuvants, such as wetting agents, emulsifying and suspending agents, and sweetening, flavoring, and perfuming agents.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Pharmaceutically acceptable carriers encompass all the foregoing and the like.

In combination therapy, administration of two or more of the therapeutic agents useful in the present invention may take place sequentially in separate formulations, or may be accomplished by simultaneous administration in a single formulation or separate formulations. Administration may be accomplished by oral route, or by intravenous, intramuscular, or subcutaneous injections. The formulation may be in the form of a bolus, or in the form of aqueous or non-aqueous isotonic sterile injection solutions or suspensions. These solutions and suspensions may be prepared from sterile powders or granules having one or more pharmaceutically-acceptable carriers or diluents, or a binder such as gelatin or hydroxypropylmethyl cellulose, together with one or more of a lubricant, preservative, surface active or dispersing agent.

For oral administration, the pharmaceutical combination may be in the form of, for example, a tablet, capsule, suspension, or liquid. Capsules, tablets, etc., can be prepared by conventional methods well known in the art. The pharmaceutical combination is preferably made in the form of a dosage unit containing a particular amount of the active ingredient or ingredients. Examples of dosage units are tablets or capsules. These may with advantage contain one or more therapeutic compound in an amount described above. For example, in the case of an HMG Co-A reductase inhibitor, the dose range may be from about 0.01 mg to about 500 mg or any other dose, dependent upon the specific inhibitor, as is known in the art.

The active ingredients may also be administered by injection as a combination wherein, for example, saline, dextrose, or water may be used as a suitable carrier. A suitable daily dose of each active therapeutic compound is one that achieves the same blood serum level as produced by oral administration as described above.

The therapeutic compounds may further be administered by any combination of oral/oral, oral/parenteral, or parenteral/parenteral route.

Pharmaceutical combinations for use in the treatment methods of the present invention may be administered in oral form or by intravenous administration. Oral administration of the combination therapy is preferred. Dosing for oral administration may be with a regimen calling for single daily dose, or for a single dose every other day, or for multiple, spaced doses throughout the day. The therapeutic compounds which make up the combination therapy may be administered simultaneously, either in a combined dosage form or in separate dosage forms intended for substantially simultaneous oral administration. The therapeutic compounds which make up the combination therapy may also be administered sequentially, with either therapeutic compound being administered by a regimen calling for two-step ingestion. Thus, a regimen may call for sequential administration of the therapeutic compounds with spaced-apart ingestion of the separate, active agents. The time period between the multiple ingestion steps may range from a few minutes to several hours, depending upon the properties of each therapeutic compound such as potency, solubility, bioavailability, plasma half-life and kinetic profile of the therapeutic compound, as well as depending upon the effect of food ingestion and the age and condition of the patient. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by intravenous route. Whether the therapeutic compounds of the combined therapy are administered by oral or intravenous route, separately or together, each such therapeutic compound will be contained in a suitable pharmaceutical formulation of pharmaceutically-acceptable excipients, diluents or other formulations components. Examples of suitable pharmaceutically-acceptable formulations containing the therapeutic compounds for oral administration are given above.

g. Treatment Regimen

The dosage regimen to prevent, give relief from, or ameliorate a disease condition having hyperlipidemia and/or inflammation as an element of the disease, e.g., atherosclerosis, or to protect against or treat plaque inflammation or high-cholesterol plasma or blood levels with the compounds and/or combinations of the present invention is selected in accordance with a variety of factors. These include the type, age, weight, sex, diet, and medical condition of the patient, the severity of the disease, the route of administration, pharmacological considerations such as the activity, efficacy, pharmacokinetics and toxicology profiles of the particular compound employed, whether a drug delivery system is utilized, and whether the compound is administered as part of a drug combination. Thus, the dosage regimen actually employed may vary widely and therefore deviate from the preferred dosage regimen set forth above.

Initial treatment of a patient suffering from a hyperlipidemic condition can begin with the dosages indicated above. Treatment should generally be continued as necessary over a period of several weeks to several months or years until the hyperlipidemic disease condition has been controlled or eliminated. Patients undergoing treatment with the compounds or combinations disclosed herein can be routinely monitored by, for example, measuring serum LDL and total cholesterol levels by any of the methods well known in the art, to determine the effectiveness of the combination therapy. Continuous analysis of such data permits modification of the treatment regimen during therapy so that optimal effective amounts of each type of therapeutic compound are administered at any point in time, and so that the duration of treatment can be determined as well. In this way, the treatment regimen/dosing schedule can be rationally modified over the course of therapy so that the lowest amount of the therapeutic compounds which together exhibit satisfactory effectiveness is administered, and so that administration is continued only so long as is necessary to successfully treat the hyperlipidemic condition.

A potential advantage of the combination therapy disclosed herein may be reduction of the amount of any individual therapeutic compound, or all therapeutic compounds, effective in treating hyperlipidemic conditions such as atherosclerosis and hypercholesterolemia.

One of the several embodiments of the present invention comprises a combination therapy comprising the use of an amount of an ASBT inhibitor and an amount of a cyclooxygenase inhibitor, wherein the amount of the ASBT inhibitor and the amount of the cyclooxygenase inhibitor together comprise an anti-hyperlipidemic condition effective amount or an anti-inflammatory condition effective amount of the ASBT inhibitor and the cyclooxygenase inhibitor. For example, one of the many embodiments of the present invention is a combination therapy comprising therapeutic dosages of an ASBT inhibitor and a cyclooxygenase-2 selective inhibitor. A preferred embodiment of the present invention is a combination therapy comprising therapeutic dosages of a benzothiepine ASBT inhibitor and a tricyclic cyclooxygenase-2 selective inhibitor.

Another embodiment of the present invention comprises a therapeutic combination containing an amount of an ASBT inhibitor, an amount of a cyclooxygenase-2 selective inhibitor or its prodrug, and a pharmaceutically acceptable carrier, wherein the amount of the ASBT inhibitor, the amount of the cyclooxygenase-2 selective inhibitor together constitute a hypercholesterolemia-related condition effective amount or an inflammation-related condition effective amount of the ASBT inhibitor and the cyclooxygenase inhibitor. For example, one of the many embodiments of the present invention is a combination comprising therapeutic dosages of an ASBT inhibitor and a COX-2 selective inhibitor. A preferred embodiment of the present invention is a combination containing therapeutic dosages of a benzothiepine ASBT inhibitor and a tricyclic COX-2 selective inhibitor.

Another embodiment of the present invention is a combination therapy comprising an amount of an ASBT inhibitor, an amount of a cyclooxygenase-2 selective inhibitor and an amount of an HMG-CoA inhibitor, wherein the amount of the ASBT inhibitor, the amount of the cyclooxygenase-2 selective inhibitor and the amount of the HMG-CoA inhibitor together constitute a hypercholesterolemia-related condition effective amount or an inflammation-related condition effective amount of the said compounds. For example, one of the many embodiments of the present invention is a combination comprising therapeutic dosages of an ASBT inhibitor, a COX-2 selective inhibitor and an HMG-CoA inhibitor. A preferred embodiment of the present invention is a combination containing therapeutic dosages of a benzothiepine ASBT inhibitor, a tricyclic COX-2 selective inhibitor and a statin HMG-CoA inhibitor.

Another embodiment of the present invention comprises a therapeutic combination containing an amount of an ASBT inhibitor, an amount of a cyclooxygenase-2 selective inhibitor or its prodrug and an amount of an HMG-CoA reductase inhibitor, and a pharmaceutically acceptable carrier, wherein the amount of the ASBT inhibitor, the amount of the cyclooxygenase-2 selective inhibitor or its prodrug and the amount of the HMG-CoA reductase inhibitor together constitute a hypercholesterolemia-related condition effective amount or an inflammation-related condition effective amount of the said compounds. For example, one of the many embodiments of the present invention is a combination comprising therapeutic dosages of an ASBT inhibitor, a COX-2 selective inhibitor and an HMG-CoA inhibitor. A preferred embodiment of the present invention is a combination containing therapeutic dosages of a benzothiepine ASBT inhibitor, a tricyclic COX-2 selective inhibitor and a statin HMG-CoA inhibitor.

In a further embodiment, the present invention provides a method for treating or preventing a hypercholesterolemia-related or an inflammation-related condition in a subject in need of such treatment or prevention, comprising treating the subject with an amount of an apical sodium co-dependent bile acid transport inhibitor, an amount of a chromene cyclooxygenase inhibitor (e.g., a chromene COX-2 selective inhibitor) or its prodrug, wherein the amount of the apical sodium co-dependent bile acid transport inhibitor, the amount of the chromene cyclooxygenase inhibitor (e.g., a chromene COX-2 selective inhibitor) together constitute a hypercholesterolemia-related condition effective amount or an inflammation-related condition effective amount of the apical sodium co-dependent bile acid transport inhibitor and the chromene cyclooxygenase inhibitor (e.g., a chromene COX-2 selective inhibitor).

In a further embodiment, the present invention provides a method for treating or preventing a hypercholesterolemia-related or an inflammation-related condition in a subject in need of such treatment or prevention, comprising treating the subject with an amount of an HMG Co-A reductase inhibitor, an amount of a chromene cyclooxygenase inhibitor (e.g., a chromene COX-2 selective inhibitor) or its prodrug, wherein the amount of the HMG Co-A reductase inhibitor and the amount of the chromene cyclooxygenase inhibitor (e.g., a chromene COX-2 selective inhibitor) together constitute a hypercholesterolemia-related condition effective amount or an inflammation-related condition effective amount of the HMG Co-A reductase inhibitor and the chromene cyclooxygenase inhibitor (e.g., a chromene COX-2 selective inhibitor).

The present invention also provides a method for treating or preventing a hypercholesterolemia-related or an inflammation-related condition in a subject in need of such treatment or prevention, comprising treating the subject with an amount of an HMG Co-A reductase inhibitor and an amount of a source of valdecoxib, wherein the amount of the HMG Co-A reductase inhibitor and the amount of the source of valdecoxib together constitute a hypercholesterolemia-related condition effective amount or an inflammation-related condition effective amount of the HMG Co-A reductase inhibitor and the source of valdecoxib. Preferably the source of valdecoxib is valdecoxib. However, the source of valdecoxib can advantageously be a prodrug of valdecoxib, for example parecoxib.

The embodiments of the present invention can comprise a combination therapy using two or more of the therapeutic compounds described or incorporated herein. The combination therapy can comprise two or more therapeutic compounds having a similar effect from different classes of chemistry, e.g., benzopyran cyclooxygenase-2 selective inhibitors can be therapeutically combined with tricyclic cyclooxygenase-2 selective inhibitors. Therapeutic combinations can also comprise more than two therapeutic compounds. For example, the therapy can comprise the use of an ASBT inhibitor, a cyclooxygenase-2 selective inhibitor, and an HMG-CoA reductase inhibitor. Alternatively, two or more compounds from the same therapeutic class of chemistry can comprise the therapy, e.g. a combination therapy comprising two or more benzothiepine ASBT inhibitors or two or more tricyclic cyclooxygenase-2 selective inhibitors.

h. Kits

The present invention further comprises kits that are suitable for use in performing the methods of treatment and/or prophylaxis described above. In one embodiment, the kit contains a first dosage form comprising one or more of the ASBT inhibitors identified in Table 2 and a second dosage form comprising a COX-2 nonselective inhibitor in quantities sufficient to carry out the methods of the present invention. In a more preferred embodiment the kit contains a first dosage form comprising one or more of the ASBT inhibitors identified in Table 2 and a second dosage form comprising a COX-2 selective inhibitor in quantities sufficient to carry out the methods of the present invention. In a still more preferred embodiment the kit contains a first dosage form comprising one or more of the ASBT inhibitors identified in Table 2 and a second dosage form comprising a COX-2 selective chromene inhibitor identified in Table 4. In an even more highly preferred embodiment the kit contains a first dosage form comprising one or more of the ASBT inhibitors identified in Table 2 and a second dosage form comprising a COX-2 selective tricyclic inhibitor identified in Tables 6 and 7A. In a particularly preferred embodiment, the kit contains a first dosage form comprising the bezothiepine ASBT inhibitor A-8 identified in Table 2 and a second dosage form comprising either celecoxib (B-18) or rofecoxib (B-21).

In another embodiment the kit contains a first dosage form comprising one or more of the ASBT inhibitors identified in Table 2 and a second dosage form comprising a COX-2 nonselective inhibitor and a third dosage form comprising an HMG-CoA reductase inhibitor in quantities sufficient to carry out the methods of the present invention. In a more preferred embodiment the kit contains a first dosage form comprising one or more of the ASBT inhibitors identified in Table 2 and a second dosage form comprising a COX-2 selective inhibitor and a third dosage form comprising an HMG-CoA reductase inhibitor in quantities sufficient to carry out the methods of the present invention. In a still more preferred embodiment the kit contains a first dosage form comprising one or more of the ASBT inhibitors identified in Table 2 and a second dosage form comprising a COX-2 selective chromene inhibitor identified in Table 4 and a third dosage form comprising an HMG-CoA reductase inhibitor. In an even more highly preferred embodiment the kit contains a first dosage form comprising one or more of the ASBT inhibitors identified in Table 2 and a second dosage form comprising a COX-2 selective tricyclic inhibitor identified in Table 6 and a third dosage form comprising an HMG-CoA reductase inhibitor. In a particularly preferred embodiment the kit comprises a first dosage form comprising the bezothiepine ASBT inhibitor A-8 identified in Table 2 and a second dosage form comprising either celecoxib (B-18) or rofecoxib (B-21) and a third dosage form comprising a statin HMG-CoA reductase inhibitor selected from the group consisting of atorvastatin, simvastatin, pravastatin, lovastatin, rosuvastatin and itavastatin.

i. Biological Assays of Utility

The utility of the combinations of the present invention can be shown by the following assays. Assays are performed in vitro and in animal models using procedures well recognized to show the utility of the present invention.

### In Vitro Assay of Compounds that Inhibit Recombinant COX-1 and/or COX-2 Activity

a. Preparation of Recombinant COX Baculoviruses

Recombinant COX-1 and COX-2 are prepared as described by Gierse et al. (J. Biochem., 305, 479-484 (1995). A 2.0 kb fragment containing the coding region of either human or murine COX-1 or human or murine COX-2 is cloned into a BamH1 site of the baculovirus transfer vector pVL1393 (Invitrogen) to generate the baculovirus transfer vectors for COX-1 and COX-2 in a manner similar to the method of D. R. O'Reilly et al. (Baculovirus Expression Vectors: A Laboratory Manual (1992). Recombinant baculoviruses are isolated by transfecting 4 pg of baculovirus transfer vector DNA into SF9 insect cells (2×10⁸) along with 200 ng of linearized baculovirus plasmid DNA by the calcium phosphate method (M. D. Summers and G. E Smith, A Manual of Methods for Baculovirus Vectors and Insect Cell Culture Procedures, Texas Agric. Exp. Station Bull. 1555 (1987)). Recombinant viruses are purified by three rounds of plaque purification, and high-titer (10⁷-10⁸ pfu/mL) stocks of virus were prepared. For large-scale production, SF9 insect cells are infected in 10-liter fermentors (0.5×10⁶/mL) with the recombinant baculovirus stock such that the multiplicity of the infection was 0.1. After 72 hours the cells are centrifuged, and the cell pellet homogenized in Tris/Sucrose (50 mM: 25%, pH 8.0) containing 1% 3-[(3)-cholamidopropyl)dimethylammonio]-1-propanesulfonate (CHAPS). The homogenate is centrifuged at 10,000×G for 30 minutes, and the resulting supernatant is stored at −80° C. before being assayed for COX activity.

b. Assay for COX-1 and COX-2 Activity

COX activity is assayed as PGE₂ formed/jg protein/time using an ELISA to detect the prostaglandin released. CHAPS-solubilized insect cell wall membranes containing the appropriate COX enzyme are incubated in a potassium phosphate buffer (50 mM, pH 8.0) containing epinephrine, phenol, and heme with the addition of arachidonic acid (10 μM). Compounds are pre-incubated with the enzyme for 10-20 minutes prior to the addition of arachidonic acid. Any reaction between the arachidonic acid and the enzyme is stopped after 10 minutes at 37° C./room temperature by transferring 40 μL of reaction mix into 160 μL ELISA buffer and 25 μM indomethacin. The PGE₂ formed will be measured by standard ELISA technology (Cayman Chemical).

c. Rapid assay for COX-1 and COX-2 Activity

COX activity is assayed as PGE₂ formed/μg protein/time using an ELISA to detect the prostaglandin released. CHAPS-solubilized insect cell wall membranes containing the appropriate COX enzyme are incubated in a potassium phosphate buffer (50 mM potassium phosphate, pH 7.5, 300 μM epinephrine, 2 μM phenol, 1 μM heme) with the addition of 20 μL of 100 μM arachidonic acid (10 μM). Compounds are pre-incubated with the enzyme for 10 minutes at 37° C. prior to the addition of arachidonic acid. Any reaction between the arachidonic acid and the enzyme is stopped after 2 minutes at 37° C./room temperature by transferring 40 μL of reaction mix into 160 μL ELISA buffer and 25 μM indomethacin. The PGE₂ formed is measured by standard, ELISA technology (Cayman Chemical).

### In Vivo Assay of Anti-inflammatory Compounds in the Rat Carageenan Foot Pad Edema Test

The carageenan foot edema test for the in vivo evaluation of anti-inflammatory potency will be as performed essentially as described by Winter et al. (Proc. Soc. Exp. Biol. Med., 111, 544 (1962). Male Sprague-Dawley rats are selected in each group having average body weights as close as possible. Rats are fasted with free access to water for over sixteen hours prior to the test. The rats are dosed orally (1 mL) with compounds suspended in vehicle containing 0.5% methylcellulose and 0.025% surfactant, or with vehicle alone. One hour later a subplantar injection of 0.1 mL of 1% solution of carrageenan/sterile 0.9% saline is administered, and the volume of the foot is measured with a displacement plethysmometer connected to a pressure transducer with a digital indicator. Three hours after the injection of the carrageenan the volume of the foot is again measured. The average foot swelling in a group of drug-treated animals is compared with that of a group of plecebo-treated animals, and the percentage inhibition of edema is determined (Otterness and Bliven, Laboratory Models for Testing NSAIDs, in Non-steroidal Anti-Inflammatory Drugs, J. Lombardino, ed., 1985).

### In Vitro Assay of Compounds that Inhibit ASBT-mediated Uptake of [¹⁴C]Taurocholate (TC) in H14 Cells

Baby hamster kidney cells (BHK) transfected with the cDNA of human ASBT (H14 cells) are seeded at 60,000 cells/well in 96-well Top-Count tissue culture plates for assays to be run within in 24 hours of seeding, at 30,000 cells/well for assays run within 48 hours, and at 10,000 cells/well for assays run within 72 hours.

On the day of assay, the cell monolayer is gently washed once with 100 μl assay buffer (Dulbecco's Modified Eagle's medium with 4.5 g/L glucose+0.2% (w/v) fatty acid free-bovine serum albumin (FAF)BSA). To each well 50 μL of a two-fold concentrate of test compound in assay buffer is added along with 50 μL of 6 μM [¹⁴C]taurocholate in assay buffer (final concentration of 3 μM [¹⁴C]taurocholate). The cell culture plates are incubated for two hours at 37° C. prior to gently washing each well twice with 100 μL of Dulbecco's phosphate-buffered saline (PBS) at 40° C. containing 0.2% (w/v) (FAF)BSA. The wells are then gently washed once with 100 μL of PBS at 4° C. without (FAF)BSA. To each well 200 μL of liquid scintillation counting fluid is added, and the plates are heat sealed and shaken for 30 minutes at room temperature prior to measuring the amount of radioactivity in each well on a Packard Top-Count instrument.

### In Vitro Assay of Compounds that Inhibit Uptake of [¹⁴C]Alanine

The alanine uptake assay is to be performed in an identical fashion to the taurocholate assay, with the exception that [¹⁴C]-labeled alanine was substituted for the radiolabelled taurocholate.

### In Vivo Assay of Compounds that Inhibit Rat Ileal Uptake of [¹⁴C]Taurocholate into Bile

(The method to be used is similar to that described by Une at al., “Metabolism of 3α,7β-dihydroxy-7α-methyl-5β-cholanoic acid and 3α,7β-dihydroxy-7α-methyl-5β-cholanoic acid in hamsters,” Biochim. Biophys. Acta, 833, 196-202 (1985).)

Male wistar rats (200-300 g) are anesthetized with inactin @100 mg/kg. Bile ducts are cannulated with a 10″ length of PE10 tubing. The small intestine is exposed and laid out on a gauze pad. A cannula (tapered female adapter with ⅛″ luer lock) is inserted at 12 cm from the junction of the small intestine and the cecum. A slit is cut at 4 cm from this same junction (utilizing a 8 cm length of ileum). Warm Dulbecco's phosphate buffered saline (PBS) at pH 6.5 (20 mL) is used to flush out the intestinal segment. The distal opening is cannulated with a 20 cm length of silicone tubing (0.020″ I.D.×0.037″ O.D.). The proximal cannula is connected to a peristaltic pump and the intestine is washed for 20 minutes with warm PBS at 0.25 mL/min. The temperature of the gut segment is monitored continuously. At the start of the experiment, 2.0 mL of control sample ([¹⁴C]taurocholate @ 0.05 mCi/mL, diluted with 5 mM unlabelled taurocholate) is loaded into the gut segment using a 3-mL syringe, and bile sample collection is begun. Control sample is infused at a rate of 0.25 mL/min for 21 minutes. Bile sample fractions are collected for radioassay every three minutes for the first 27 minutes of the procedure. After 21 minutes of sample infusion, the ileal loop is washed out with 20 mL of warm PBS (using a 30-mL syringe), and the loop is further washed out for 21 minutes with warm PBS at 0.25 mL/min. A second perfusion is then initiated as described above, but with test compound being simultaneously administered as well (21 minutes of administration followed by 21 minutes of washout), and bile is sampled every 3 minutes for the first 27 minutes. If necessary, a third perfusion is performed as above using the control sample.

### Measurement of Rat Hepatic Cholesterol Concentration (HEPATIC CHOL)

Rat liver tissue is weighed and homogenized in chloroform:methanol (2:1). After homogenization and centrifugation the supernatant is separated and dried under nitrogen. The residue is dissolved in isopropanol and the cholesterol content is measured enzymatically, using a combination of cholesterol oxidase and peroxidase, as described by Allain et al., Clin. Chem., 20, 470 (1974).

### Measurement of Rat Hepatic HMG-CoA Reductase Activity

Rat liver microsomes are prepared by homogenizing liver samples in a phosphate/sucrose buffer, followed by centrifugal separation. The final pelleted material is resuspended in buffer and an aliquot is assayed for HMG-CoA reductase activity by incubating for 60 minutes at 37° C. in the presence of [¹⁴C]HMG-CoA (Dupont-NEN). The reaction is stopped by adding 6N HCl followed by centrifugation. An aliquot of the supernatant is subjected to separation using thin-layer chromatography, and the spot corresponding to the enzymatic product is scraped off the plate, extracted and assayed for radioactivity by scintillation counting (Akerlund and Bjorkhem, J. Lipid Res., 31, 2159 (1990).

### Determination of Rat Serum Cholesterol (SER.CHOL, HDL-CHOL, TGI and VLDL+LDL)

Total rat serum cholesterol (SER.CHOL) is measured enzymatically using a commercial kit from Wako Fine Chemicals (Richmond, Va.); Cholesterol C11, Catalog No. 276-64909. HDL cholesterol (HDL-CHOL) is assayed using this same kit after precipitation of VLDL and LDL with Sigma Chemical Co. HDL cholesterol reagent, Catalog No. 352-3 (dextran sulfate method). Total serum triglycerides (blanked) (TGI) are assayed enzymatically with Sigma Chemical Co. GPO-Trinder, Catalog No. 337-B. VLDL and LDL (VLDL+LDL) cholesterol concentrations are calculated as the difference between total and HDL cholesterol.

### Measurement of Rat Hepatic Cholesterol 7-α-Hydroxylase Activity (7α-HOase)

Rat liver microsolnes are prepared by homogenizing liver samples in a phosphate/sucrose buffer, followed by centrifugal separation. The final pelleted material is resuspended in buffer and an aliquot is assayed for cholesterol 7-α-hydroxylase activity by incubating for 5 minutes at 37° C. in the presence of NADPH. Following extraction into petroleum ether, the organic solvent is evaporated and the residue is dissolved in acetonitrile/methanol. The enzymatic product will be separated by injecting an aliquot of the extract onto a C₁₈ reverse-phase HPLC column and quantitating the eluted material using UV detection at 240 nm. (Horton et al., J. Clin. Invest., 93, 2084 (1994)).

### In Vivo Rat Gavage ASBT Assay

Male Wister rats (275-300 g) are administered ASBT inhibitors using an oral gavage procedure. Drug or vehicle (0.2% Tween 80 in water) is administered once a day (9:00-10:0 a.m.) for 4 days at varying dosages in a final volume of 2 mL per kilogram of body weight. Total fecal samples are collected during the final 48 hours of the treatment period and analyzed for bile acid content using an enzymatic assay as described below. Compound efficacy is determined by comparison of the increase in fecal bile acid (FBA) concentration in treated rats to the mean FBA concentration of rats in the vehicle group.

### Measurement of Hamster Fecal Bile Acid Concentration (FBA)

Total fecal output from individually housed hamsters is collected for 24 or 48 hours, dried under a stream of nitrogen, pulverized and weighed. Approximately 0.1 gram is weighed out and extracted using an organic solvent (butanol/water). Following separation and drying, the residue is dissolved in methanol and the amount of bile acid present is measured enzymatically using the 3α-hydroxysteroid steroid dehydrogenase reaction with bile acids to reduce NAD. (Mashige et al. Clin. Chem., 27, 1352 (1981)).

### [³H]Taurocholate uptake in Rabbit Brush Border Membrane Vesicles (BBMV)

Rabbit ileal brush border membranes are prepared from frozen ileal mucosa by the calcium precipitation method describe by Malathi et al. (Biochim. Biophys. Acta, 554, 259 (1979). The method for measuring taurocholate is similar to that described by Kramer et al. (Biochim. Biophys. Acta, 1111, 93 (1992)) except that the assay volume used is 200 μL instead of 100 μL. Briefly, at room temperature a 190-μl solution containing 2 μM [³H]taurocholate(0.75 μCi), 20 mM tris, 100 mm NaCl, 100 mM mannitol, pH 7.4, is incubated for 5 seconds with 10 μL of brush border membrane vesicles (60-120 μg protein). The incubation is initiated by the addition of the BBMV while vortexing and the reaction is quenched by the addition of 5 mL of ice-cold buffer (20 mM Hepes-tris, 150 mM KCl), followed immediately by filtration through a nylon filter (0.2 μm porosity) and washing with an additional 5 mL of quench buffer.

### Dog Model for the Evaluation of Lipid-lowering Drugs (e.g., an ASBT Inhibitor or an HMG Co-A Reductase Inhibitor)

Male beagle dogs weighing 6-12 kg, are fed once a day for two hours and given water ad libitum. Dogs are randomly assigned to dosing groups consisting of 6 to 12 dogs each, corresponding to: vehicle, i.g.; 1 mg/kg, i.g.; 2 mg/kg, i.g.; 4 mg/kg, i.g.; 2 mg/kg, p.o. (powder in capsule). Intra-gastric dosing of a therapeutic compound dissolved in aqueous solution (for example, 0.2% Tween 80 solution [polyoxyethylene mono-oleate, Sigma Chemical Co., St. Louis, Mo.]) is performed using a gavage tube. Prior to initiation of dosing, blood samples are drawn from the cephalic vein before the morning feeding in order to evaluate serum cholesterol (total and HDL) and triglycerides. For several consecutive days animals are dosed in the morning prior to feeding. Animals are thereafter allowed to eat for two hours before remaining food was removed. Feces are collected over a 2-day period at the end of the study and were analyzed for bile acid or lipid content. Blood samples are also collected at the end of the treatment period for comparison with pre-study serum lipid levels. Statistical significance will be determined using the standard Student's T-test, with p<0.05.

### Dog Serum Lipid Measurement

Blood is collected from the cephalic veins of fasted dogs using serum separator tubes (Vacutainer SST, Becton Dickinson and Co., Franklin Lakes, N.J.). The blood is centrifuged at 2000 rpm for 20 minutes and the serum decanted.

Total cholesterol is measured in a 96-well format using a Wako enzymatic diagnostic kit (Cholesterol CII) (Wako Chemicals, Richmond, Va.), utilizing the cholesterol oxidase reaction to produce hydrogen peroxide, which is measured calorimetrically. A standard curve from 0.5 to 10 μg cholesterol is prepared in the first two columns of the plate. The serum samples (20-40 μL, depending on the expected lipid concentration) or known serum control samples were added to individual wells in duplicate. Water is added to bring the volume to 100 μL in each well. A 100-μl aliquot of color reagent is added to each well, and the plates are read at 500 nm after a 15-minute incubation at 37° C.

HDL cholesterol is assayed using Sigma kit No. 352-3 (Sigma Chemical Co., St. Louis, Mo.), which utilizes dextran sulfate and Mg²⁺ to selectively precipitate LDL and VLDL. A volume of 150 μL of each serum sample is added to individual microfuge tubes, followed by 15 μL of HDL cholesterol reagent (Sigma 352-3). Samples are mixed and centrifuged at 5000 rpm for 5 minutes. A 50 μL aliquot of the supernatant is then mixed with 200 μL of saline and assayed using the same procedure as for total cholesterol measurement.

Triglycerides is measured using Sigma kit No. 337 in a 96-well plate format. This procedure measures the release glycerol from triglycerides with lipoprotein lipase. Standard solutions of glycerol (Sigma 339-11) ranging from 1 to 24 μg are used to generate the standard curve. Serum samples (20-40 μL, depending on the expected lipid concentration) are added to wells in duplicate. Water is added to bring the volume to 100 μL in each well and 100 μL of color reagent is also added to each well. After mixing and a 15-minute incubation, the plates will be read at 540 nm and the triglyceride values will be calculated from the standard curve. A replicate plate also will be run using a blank enzyme reagent to correct for any endogenous glycerol in the serum samples.

### Dog Fecal Bile Acid Measurement

Fecal samples are collected to determine the fecal bile acid (FBA) concentration for each animal. Fecal collections are made during the final 48 hours of the study, for two consecutive 24-hour periods between 9:00 a.m. and 10:00 a.m. each day, prior to dosing and feeding. The separate two-day collections from each animal are weighed, combined and homogenized with distilled water in a processor (Cuisinart) to generate a homogeneous slurry. A sample of 1.4 g of the homogenate is extracted in a final concentration of 50% tertiary butanol/distilled water (2:0.6) for 45 minutes in a 37° water bath and centrifuged for 13 minutes at 2000×G. The concentration of bile acids (mmoles/day) is determined using a 96-well enzymatic assay system. A 20-μL aliquot of the fecal extract is added to two sets each of triplicate wells in a 96-well assay plate. A standardized sodium taurocholate solution and a standardized fecal extract solution (previously made from pooled samples and characterized for its bile acid concentration) are also analyzed for assay quality control. Aliquots of sodium taurocholate (20 μL), serially diluted to generate a standard curve, are similarly added to two sets of triplicate wells. A 230-μL reaction mixture containing 1M hydrazine hydrate, 0.1 M pyrophosphate and 0.46 mg/ml NAD is added to each well. A 50-μL aliquot of 3α-hydroxysteroid dehydrogenase enzyme (HSD; 0.8 units/ml) or assay buffer (0.1 M sodium pyrophosphate) is then added to one of the two sets of triplicates. All reagents are obtained from Sigma Chemical Co., St. Louis, Mo. Following 60 minutes of incubation at room temperature, the optical density at 340 nm is measured and the mean of each set of triplicate samples was calculated. The difference in optical density ±HSD enzyme is used to determine the bile acid concentration (mM) of each sample, based on the'sodium taurocholate standard curve. The bile acid concentration of the extract, the weight of the fecal homogenate (grams) and the body weight of the animal is used to calculate the corresponding FBA concentration in mmoles/kg/day for each animal. The mean FBA concentration (mmoles/kg/day) of the vehicle group is subtracted from the FBA concentration of each treatment group to determine the increase (delta value) in FBA concentration as a result of the treatment.

### Hamster Intestinal Cholesterol Absorption Assay

Various compounds can be shown to inhibit cholesterol absorption from the intestinal tract. These compounds lower serum cholesterol levels by reducing intestinal absorption of cholesterol from both exogenous sources (dietary cholesterol) and endogenous cholesterol (secreted by the gall bladder into the intestinal tract).

In hamsters the use of a dual-isotope plasma ratio method to measure intestinal cholesterol absorption will be refined and evaluated as described by Turley et al. (J. Lipid Res., 35, 329-339 (1994)).

Male hamsters weighing 80-100 g are given food and water ad libitum in a room with 12-hour alternating periods of light and dark. Four hours into the light period, each hamster is administered an intravenous dose of 2.5 μCi of [1,2-³H]cholesterol suspended in Intralipid (20%), followed by an oral dose of [4-¹⁴C]cholesterol in an oil vehicle containing medium-chain triglycerides (MCT). The i.v. dose is given by injecting a 0.4-mL volume of the Intralipid mixture into the distal femoral vein. The oral dose is given by gavaging a 0.6-mL volume of the MCT oil mixture intragastrically via a polyethylene tube. After 72 hours the hamsters are bled and the amount of [³H] and [¹⁴C] in the plasma and in the original radiolabelled dosing mixtures are determined by liquid scintillation spectrometry. The cholesterol absorption is calculated from the following equation:

$$\text{Percent cholestrol absorbed} = \frac{\text{\% of oral dose per mL of 72-hour plasma sample}}{\text{\% of i.v. dose per mL of 72-hour plasma sample}} \times 100$$

### Evaluation of Plasma Lipids and Atherosclerotic Lesions in Rabbits

Rabbit plasma lipids are assayed using standard methods as reported by Schuh et al., J. Clin. Invest., 91, 1453-1458 (1993). Groups of male New Zealand white rabbits are placed on a standard diet (100 g/day) supplemented with 0.3% cholesterol and 2% corn oil (Zeigler Bothers, Inc., Gardners, Pa.). Water is available ad libitum. Groups of control and treated animals are sacrificed after one and three months of treatment. Blood samples are collected for determination of plasma lipid concentrations. Tissues are removed for characterization of atherosclerotic lesions and aorta vascular response.

a. Plasma Lipids

Plasma for lipid analysis is obtained by withdrawing blood from the ear vein into EDTA-containing tubes (Vacutainer; Becton Dickenson & Co., Rutherford, N.J.), followed by centrifugation of the cells. Total cholesterol is determined enzymatically, using the cholesterol oxidase reaction (C. A. Allain et al., Clin. Chem., 20, 470-475 (1974)). HDL cholesterol is also measured enzymatically, after selective precipitation of LDL and VLDL by dextran sulfate with magnesium (Warnick et al., Clin. Chem., 28, 1379-1388 (1982)). Plasma triglyceride levels are determined by measuring the amount of glycerol released by lipoprotein lipase through an enzyme-linked assay (G. Bucolo et al., Clin. Chem., 19, 476-482 (1973)).

b. Atherosclerotic Lesions

Animals are sacrificed by pentobarbital injection. Thoracic aortas are rapidly removed and fixed by immersion in 10% neutral buffered formalin, and stained with oil red O (0.3%). After a single longitudinal incision along the wall opposite the arterial ostia, the vessels are pinned open for evaluation of the plaque area. The percent plaque coverage is determined from the values for the total area examined and the stained area by threshold analysis using a true color image analyzer (Videometric 150; American Innovision, Inc., San Diego, Calif.) interfaced to a color camera (Toshiba 3CCD) mounted on a dissecting microscope. Tissue cholesterol is measured enzymatically as previously described, after extraction with a chloroform/methanol mixture (2:1, according to the method of Folch et al. (J. Biol. Chem., 226, 497-509 (1957)).

c. Aorta Vascular Response

The abdominal aortas are rapidly excised after injection of sodium pentobarbital and placed in oxygenated Krebs-bicarbonate buffer. After removal of perivascular tissue, 3-mm ring segments are cut, placed in a 37° C. muscle bath containing Krebs-bicarbonate solution, and suspended between two stainless steel wires, one of which is attached to a force transducer (Grass Instrument Co., Quincy, Mass.). Force changes in response to angiotensin II added to the bath will be recorded on a chart recorder.

### Evaluation of Plasma Lipids and Atherosclerotic Lesions in Mouse Models of Atherosclerosis

Male LDL receptor (−/−) mice (6-8 weeks of age) are obtained from the Jackson Laboratories (Bar Harbor, Me.) and are permitted an acclimatization period of one week on normal diet. Mice are then placed on a diet enriched in saturated fat (21% wt/wt) and cholesterol (0.15% wt/wt; Harlan Teklad, catalog #88137). Pelleted diets are prepared by Research Diets, New Brunswick, N.J. Compounds are administered by mixing the drug in the diet at the indicated concentrations. On occasion, drugs can be administered in the drinking water. Mice are maintained on the above regimens for a minimum of 8 weeks and usually a total of 12 weeks.

Male ApoE (−/−) mice are obtained from the Jackson Laboratories (Bar Harbor, Me.) and are permitted an acclimatization period of one week on normal diet. Mice (6 weeks of age) are then placed on a normal chow diet (Purina Certified 5002 Diet) or on a saturated fat (21% wt/wt) and cholesterol (0.15% wt/wt; Harlan Teklad, catalog #88137) to accelerate the rate of atherosclerosis formation. Pelleted diets are prepared by Research Diets, New Brunswick, N.J. Compounds are administered by mixing the drug in the diet at the indicated concentrations. Mice are maintained on the above regimens for a minimum of 8 weeks and usually a total of 12 weeks.

a. Lipid Analyses

Serum cholesterol concentrations were determined by enzymatic assay and lipoprotein-cholesterol distribution was determined by size exclusion chromatography as described previously (Daugherty A and Rateri D, Coronary Artery Dis. 2: 775-787 (1991).

b. Quantification and Histological Analyses of the Atherosclerotic Lesions

The extent of the aortic intima covered by grossly discernable atherosclerotic lesions can be quantified by en face analysis of the aorta (from the top of the heart to the iliac bifurcation) as described previously (Daugherty A et al. J. Clin. Invest.100:1575-1580 (1997); Daugherty A at al. J. Clin. Invest. 105:1605-1612 (2000).

Alternatively, atherosclerotici lesion area can be determined in the aortic roots of animals which correlates extremely well with en face atherosclerotic lesion area assessment, but allows histologc evaluation of the quality of the lesions themselves. Mice are euthanized with CO₂ gas and blood is removed by retroorbital collection. Hearts are immediately removed and fixed in phosphate buffered formalin. After 24 hours, the bottom two-thirds of the hearts are removed by carefully sectioning the heart just below the atria. The remaining top portions of the hearts are embedded in paraffin and 4 μm sections are cut. Every 6th section is evaluated for cross sectional area of atherosclerotic lesions by hematoxylin and eosin staining, beginning where the atrial valves appeared distinctly to where the valves disappear, as described earlier by Nishina et al. (Nishina PM et al, Lipids 28: 599-605 (1993).

Serial sections of the proximal aorta, within 50 microns of the valves and containing remnants of the valve leaflets are selected for immunolocalization of lymphocytes, (anti-CD3), macrophages (anti-CD1) and smooth muscle cells (SMA) and counterstained using hemotoxylin or methyl green. All lesions contained within one aortic section per individual are evaluated. Lesions are characterized as early (Stary classification I and II) or complex (Stary classification III and IV).

T cell quantification in atherosclerotic lesions is performed on sections stained with an anti-CD3 antibody followed by digital image analysis on a computer controlled Olympus AX-70 Provis microscope equipped with a Photometrix digital camera, liquid crystal tunable filter and Isee Imaging software (Inovison Corp, Raleigh, N.C.). Procedures for image acquisition and image analysis has been previously described (Ornberg RL. J. Histochem. Cytochem. 49:1059-1060 (2000); Ornberg RL et al. Journal of Histochemistry and Cytochemistry. 47(9): 1-7 (1999).

For smooth muscle cell content, aortic root section images were captured using a Zeiss Axiophot equipped with a Spot XX camera and a 10× objective with a 1.6× magnification ring. Lesion area positively stained for SMA was measured by selecting threshold criteria to detect 1% of a negative control tissue (lymph node) and >85% of a positive control, which was typically a normal media. All lesions are included in the analysis; early or complex lesion assignment is noted during data capture. All measurements are performed by blinded observers and analyzed with measured Area of smooth muscle actin by quantitative image analysis Optimus 6.1.3.

c. Statistical Analyses

Statistically significant differences among the means of different groups are tested using one-way analysis of variance (ANOVA).

### j. EXAMPLES OF EMBODIMENTS

The following non-limiting examples serve to illustrate various aspects of the present invention.

### Example 1

### Pharmaceutical Compositions

100 mg tablets of the composition set forth in Table X-1 can be prepared using wet granulation techniques:

TABLE X-1

| Ingredient | Weight (mg) |
|---|---|
| Compound A-7 (Benzothiepine) | 5 |
| Compound B-18 (Celecoxib) | 20 |
| Lactose | 54 |
| Microcrystalline Cellulose | 15 |
| Hydroxypropyl Methylcellulose | 3 |
| Croscarmelose Sodium | 2 |
| Magnesium Stearate | 1 |
| Total Tablet Weight | 100 |

### Example 2

### Pharmaceutical Compositions

100 mg tablets of the composition set forth in Table X-2 can be prepared using direct compression techniques:

TABLE X-2

| Ingredient | Weight (mg) |
|---|---|
| Compound A-7 (Benzothiepine) | 5 |
| Compound B-18 (Celecoxib) | 20 |
| Microcrystalline Cellulose | 69.5 |
| Colloidal Silicon Dioxide | 0.5 |
| Talc | 2.5 |
| Croscarmelose Sodium | 2 |
| Magnesium Stearate | 0.5 |
| Total Tablet Weight | 100 |

Combinations

Tables X-3 and X-3A illustrate, by way of example and not limitation, some of the many combinations of the present invention wherein the combination comprises an amount of an ASBT inhibitor (Component 1) and an amount of a cyclooxygenase-2 selective inhibitor (Component 2), wherein the amount of the ASBT inhibitor and the amount of the cyclooxygenase-2 selective inhibitor together constitute a hypercholesterolemia-related condition effective amount or an inflammation-related condition effective amount of the ASBT inhibitor and the cyclooxygenase-2 selective inhibitor.

TABLE X-3

| Example Number | Component 1 | Component 2 |
|---|---|---|
| 1x | A-3 | B-18 |
| 2x | A-3 | B-19 |
| 3x | A-3 | B-20 |
| 4x | A-3 | B-21 |
| 5x | A-3 | B-22 |
| 6x | A-3 | B-23 |
| 7x | A-3 | B-24 |
| 8x | A-5 | B-18 |
| 9x | A-5 | B-19 |
| 10x | A-5 | B-20 |
| 11x | A-5 | B-21 |
| 12x | A-5 | B-22 |
| 13x | A-5 | B-23 |
| 14x | A-5 | B-24 |
| 15x | A-7 | B-18 |
| 16x | A-7 | B-19 |
| 17x | A-7 | B-20 |
| 18x | A-7 | B-21 |
| 19x | A-7 | B-22 |
| 20x | A-7 | B-23 |
| 21x | A-7 | B-24 |

TABLE X-3A

| Example Number | Component 1 | Component 2 |
|---|---|---|
| 22X | A-3 | D-1 |
| 23X | A-3 | D-2 |
| 24X | A-3 | D-3 |
| 25X | A-3 | D-4 |
| 26X | A-3 | D-5 |
| 27X | A-3 | D-6 |
| 28X | A-3 | D-7 |
| 29X | A-3 | D-8 |
| 30X | A-3 | D-9 |
| 31X | A-3 | D-10 |
| 32X | A-3 | D-11 |
| 33X | A-3 | D-12 |
| 34X | A-3 | D-13 |
| 35X | A-3 | D-14 |
| 36X | A-3 | D-15 |
| 37X | A-3 | D-16 |
| 38X | A-3 | D-17 |
| 39X | A-3 | D-18 |
| 40X | A-3 | D-19 |
| 41X | A-3 | D-20 |
| 42X | A-3 | D-21 |
| 43X | A-3 | D-22 |
| 44X | A-3 | D-23 |
| 45X | A-3 | D-24 |
| 46X | A-3 | D-25 |
| 47X | A-3 | D-26 |
| 48X | A-3 | D-27 |
| 49X | A-3 | D-28 |
| 50X | A-3 | D-29 |
| 51X | A-3 | D-30 |
| 52X | A-3 | D-31 |
| 53X | A-3 | D-32 |
| 54X | A-3 | D-33 |
| 55X | A-3 | D-34 |
| 56X | A-3 | D-35 |
| 57X | A-3 | D-36 |
| 58X | A-3 | D-37 |
| 59X | A-3 | D-38 |
| 60X | A-3 | D-39 |
| 61X | A-3 | D-40 |
| 62X | A-3 | D-41 |
| 63X | A-3 | D-42 |
| 64X | A-3 | D-43 |
| 65X | A-3 | D-44 |
| 66X | A-3 | D-45 |
| 67X | A-3 | D-46 |
| 68X | A-3 | D-47 |
| 69X | A-3 | D-48 |
| 70X | A-3 | D-49 |
| 71X | A-3 | D-50 |
| 72X | A-3 | D-51 |
| 73X | A-3 | D-52 |
| 74X | A-3 | D-53 |
| 75X | A-3 | D-54 |
| 76X | A-3 | D-55 |
| 77X | A-3 | D-56 |
| 78X | A-3 | D-57 |
| 79X | A-3 | D-58 |
| 80X | A-3 | D-59 |
| 81X | A-3 | D-60 |
| 82X | A-3 | D-61 |
| 83X | A-3 | D-62 |
| 84X | A-3 | D-63 |
| 85X | A-3 | D-64 |
| 86X | A-3 | D-65 |
| 87X | A-3 | D-66 |
| 88X | A-3 | D-67 |
| 89X | A-3 | D-68 |
| 90X | A-3 | D-69 |
| 91X | A-3 | D-70 |
| 92X | A-3 | D-71 |
| 93X | A-3 | D-72 |
| 94X | A-3 | D-73 |
| 95X | A-3 | D-74 |
| 96X | A-3 | D-75 |
| 97X | A-3 | D-76 |
| 98X | A-3 | D-77 |
| 99X | A-3 | D-78 |
| 100X | A-3 | D-79 |
| 101X | A-3 | D-80 |
| 102X | A-3 | D-81 |
| 103X | A-3 | D-82 |
| 104X | A-3 | D-83 |
| 105X | A-3 | D-84 |
| 106X | A-3 | D-85 |
| 107X | A-3 | D-86 |
| 108X | A-3 | D-87 |
| 109X | A-3 | D-88 |
| 110X | A-3 | D-89 |
| 111X | A-3 | D-90 |
| 112X | A-3 | D-91 |
| 113X | A-3 | D-92 |
| 114X | A-3 | D-93 |
| 115X | A-3 | D-94 |
| 116X | A-3 | D-95 |
| 117X | A-3 | D-96 |
| 118X | A-3 | D-97 |
| 119X | A-3 | D-98 |
| 120X | A-3 | D-99 |
| 121X | A-3 | D-100 |
| 122X | A-3 | D-101 |
| 123X | A-3 | D-102 |
| 124X | A-3 | D-103 |
| 125X | A-3 | D-104 |
| 126X | A-3 | D-105 |
| 127X | A-3 | D-106 |
| 128X | A-3 | D-107 |
| 129X | A-3 | D-108 |
| 130X | A-3 | D-109 |
| 131X | A-3 | D-110 |
| 132X | A-3 | D-111 |
| 133X | A-3 | D-112 |
| 134X | A-3 | D-113 |
| 135X | A-3 | D-114 |
| 136X | A-3 | D-115 |
| 137X | A-3 | D-116 |
| 138X | A-3 | D-117 |
| 139X | A-3 | D-118 |
| 140X | A-3 | D-119 |
| 141X | A-3 | D-120 |
| 142X | A-3 | D-121 |
| 143X | A-3 | D-122 |
| 144X | A-3 | D-123 |
| 145X | A-3 | D-124 |
| 146X | A-3 | D-125 |
| 147X | A-3 | D-126 |
| 148X | A-3 | D-127 |
| 149X | A-3 | D-128 |
| 150X | A-3 | D-129 |
| 151X | A-3 | D-130 |
| 152X | A-3 | D-131 |
| 153X | A-3 | D-132 |
| 154X | A-3 | D-133 |
| 155X | A-3 | D-134 |
| 156X | A-3 | D-135 |
| 157X | A-3 | D-136 |
| 158X | A-3 | D-137 |
| 159X | A-3 | D-138 |
| 160X | A-3 | D-139 |
| 161X | A-3 | D-140 |
| 162X | A-3 | D-141 |
| 163X | A-3 | D-142 |
| 164X | A-3 | D-143 |
| 165X | A-3 | D-144 |
| 166X | A-3 | D-145 |
| 167X | A-3 | D-146 |
| 168X | A-3 | D-147 |
| 169X | A-3 | D-148 |
| 170X | A-3 | D-149 |
| 171X | A-3 | D-150 |
| 172X | A-3 | D-151 |
| 173X | A-3 | D-152 |
| 174X | A-3 | D-153 |
| 175X | A-3 | D-154 |
| 176X | A-3 | D-155 |
| 177X | A-3 | D-156 |
| 178X | A-3 | D-157 |
| 179X | A-3 | D-158 |
| 180X | A-3 | D-159 |
| 181X | A-3 | D-160 |
| 182X | A-3 | D-161 |
| 183X | A-3 | D-162 |
| 184X | A-3 | D-163 |
| 185X | A-3 | D-164 |
| 186X | A-3 | D-165 |
| 187X | A-3 | D-166 |
| 188X | A-3 | D-167 |
| 189X | A-3 | D-168 |
| 190X | A-3 | D-169 |
| 191X | A-3 | D-170 |
| 192X | A-3 | D-171 |
| 193X | A-3 | D-172 |
| 194X | A-3 | D-173 |
| 195X | A-3 | D-174 |
| 196X | A-3 | D-175 |
| 197X | A-3 | D-176 |
| 198X | A-3 | D-177 |
| 199X | A-3 | D-178 |
| 200X | A-3 | D-179 |
| 201X | A-3 | D-180 |
| 202X | A-3 | D-181 |
| 203X | A-3 | D-182 |
| 204X | A-3 | D-183 |
| 205X | A-3 | D-184 |
| 206X | A-3 | D-185 |
| 207X | A-3 | D-186 |
| 208X | A-3 | D-187 |
| 209X | A-3 | D-188 |
| 210X | A-3 | D-189 |
| 211X | A-3 | D-190 |
| 212X | A-3 | D-191 |
| 213X | A-3 | D-192 |
| 214X | A-3 | D-193 |
| 215X | A-3 | D-194 |
| 216X | A-3 | D-195 |
| 217X | A-3 | D-196 |
| 218X | A-3 | D-197 |
| 219X | A-3 | D-198 |
| 220X | A-3 | D-199 |
| 221X | A-3 | D-200 |
| 222X | A-3 | D-201 |
| 223X | A-3 | D-202 |
| 224X | A-3 | D-203 |
| 225X | A-3 | D-204 |
| 226X | A-3 | D-205 |
| 227X | A-3 | D-206 |
| 228X | A-3 | D-207 |
| 229X | A-3 | D-208 |
| 230X | A-3 | D-209 |
| 231X | A-3 | D-210 |
| 232X | A-3 | D-211 |
| 233X | A-3 | D-212 |
| 234X | A-3 | D-213 |
| 235X | A-3 | D-214 |
| 236X | A-3 | D-215 |
| 237X | A-3 | D-216 |
| 238X | A-3 | D-217 |
| 239X | A-3 | D-218 |
| 240X | A-3 | D-219 |
| 241X | A-3 | D-220 |
| 242X | A-3 | D-221 |
| 243X | A-3 | D-222 |
| 244X | A-3 | D-223 |
| 245X | A-3 | D-224 |
| 246X | A-3 | D-225 |
| 247X | A-3 | D-226 |
| 248X | A-3 | D-227 |
| 249X | A-3 | D-228 |
| 250X | A-3 | D-229 |
| 251X | A-3 | D-230 |
| 252X | A-3 | D-231 |
| 253X | A-3 | D-232 |
| 254X | A-5 | D-1 |
| 255X | A-5 | D-2 |
| 256X | A-5 | D-3 |
| 257X | A-5 | D-4 |
| 258X | A-5 | D-5 |
| 259X | A-5 | D-6 |
| 260X | A-5 | D-7 |
| 261X | A-5 | D-8 |
| 262X | A-5 | D-9 |
| 263X | A-5 | D-10 |
| 264X | A-5 | D-11 |
| 265X | A-5 | D-12 |
| 266X | A-5 | D-13 |
| 267X | A-5 | D-14 |
| 268X | A-5 | D-15 |
| 269X | A-5 | D-16 |
| 270X | A-5 | D-17 |
| 271X | A-5 | D-18 |
| 272X | A-5 | D-19 |
| 273X | A-5 | D-20 |
| 274X | A-5 | D-21 |
| 275X | A-5 | D-22 |
| 276X | A-5 | D-23 |
| 277X | A-5 | D-24 |
| 278X | A-5 | D-25 |
| 279X | A-5 | D-26 |
| 280X | A-5 | D-27 |
| 281X | A-5 | D-28 |
| 282X | A-5 | D-29 |
| 283X | A-5 | D-30 |
| 284X | A-5 | D-31 |
| 285X | A-5 | D-32 |
| 286X | A-5 | D-33 |
| 287X | A-5 | D-34 |
| 288X | A-5 | D-35 |
| 289X | A-5 | D-36 |
| 290X | A-5 | D-37 |
| 291X | A-5 | D-38 |
| 292X | A-5 | D-39 |
| 293X | A-5 | D-40 |
| 294X | A-5 | D-41 |
| 295X | A-5 | D-42 |
| 296X | A-5 | D-43 |
| 297X | A-5 | D-44 |
| 298X | A-5 | D-45 |
| 299X | A-5 | D-46 |
| 300X | A-5 | D-47 |
| 301X | A-5 | D-48 |
| 302X | A-5 | D-49 |
| 303X | A-5 | D-50 |
| 304X | A-5 | D-51 |
| 305X | A-5 | D-52 |
| 306X | A-5 | D-53 |
| 307X | A-5 | D-54 |
| 308X | A-5 | D-55 |
| 309X | A-5 | D-56 |
| 310X | A-5 | D-57 |
| 311X | A-5 | D-58 |
| 312X | A-5 | D-59 |
| 313X | A-5 | D-60 |
| 314X | A-5 | D-61 |
| 315X | A-5 | D-62 |
| 316X | A-5 | D-63 |
| 317X | A-5 | D-64 |
| 318X | A-5 | D-65 |
| 319X | A-5 | D-66 |
| 320X | A-5 | D-67 |
| 321X | A-5 | D-68 |
| 322X | A-5 | D-69 |
| 323X | A-5 | D-70 |
| 324X | A-5 | D-71 |
| 325X | A-5 | D-72 |
| 326X | A-5 | D-73 |
| 327X | A-5 | D-74 |
| 328X | A-5 | D-75 |
| 329X | A-5 | D-76 |
| 330X | A-5 | D-77 |
| 331X | A-5 | D-78 |
| 332X | A-5 | D-79 |
| 333X | A-5 | D-80 |
| 334X | A-5 | D-81 |
| 335X | A-5 | D-82 |
| 336X | A-5 | D-83 |
| 337X | A-5 | D-84 |
| 338X | A-5 | D-85 |
| 339X | A-5 | D-86 |
| 340X | A-5 | D-87 |
| 341X | A-5 | D-88 |
| 342X | A-5 | D-89 |
| 343X | A-5 | D-90 |
| 344X | A-5 | D-91 |
| 345X | A-5 | D-92 |
| 346X | A-5 | D-93 |
| 347X | A-5 | D-94 |
| 348X | A-5 | D-95 |
| 349X | A-5 | D-96 |
| 350X | A-5 | D-97 |
| 351X | A-5 | D-98 |
| 352X | A-5 | D-99 |
| 353X | A-5 | D-100 |
| 354X | A-5 | D-101 |
| 355X | A-5 | D-102 |
| 356X | A-5 | D-103 |
| 357X | A-5 | D-104 |
| 358X | A-5 | D-105 |
| 359X | A-5 | D-106 |
| 360X | A-5 | D-107 |
| 361X | A-5 | D-108 |
| 362X | A-5 | D-109 |
| 363X | A-5 | D-110 |
| 364X | A-5 | D-111 |
| 365X | A-5 | D-112 |
| 366X | A-5 | D-113 |
| 367X | A-5 | D-114 |
| 368X | A-5 | D-115 |
| 369X | A-5 | D-116 |
| 370X | A-5 | D-117 |
| 371X | A-5 | D-118 |
| 372X | A-5 | D-119 |
| 373X | A-5 | D-120 |
| 374X | A-5 | D-121 |
| 375X | A-5 | D-122 |
| 376X | A-5 | D-123 |
| 377X | A-5 | D-124 |
| 378X | A-5 | D-125 |
| 379X | A-5 | D-126 |
| 380X | A-5 | D-127 |
| 381X | A-5 | D-128 |
| 382X | A-5 | D-129 |
| 383X | A-5 | D-130 |
| 384X | A-5 | D-131 |
| 385X | A-5 | D-132 |
| 386X | A-5 | D-133 |
| 387X | A-5 | D-134 |
| 388X | A-5 | D-135 |
| 389X | A-5 | D-136 |
| 390X | A-5 | D-137 |
| 391X | A-5 | D-138 |
| 392X | A-5 | D-139 |
| 393X | A-5 | D-140 |
| 394X | A-5 | D-141 |
| 395X | A-5 | D-142 |
| 396X | A-5 | D-143 |
| 397X | A-5 | D-144 |
| 398X | A-5 | D-145 |
| 399X | A-5 | D-146 |
| 400X | A-5 | D-147 |
| 401X | A-5 | D-148 |
| 402X | A-5 | D-149 |
| 403X | A-5 | D-150 |
| 404X | A-5 | D-151 |
| 405X | A-5 | D-152 |
| 406X | A-5 | D-153 |
| 407X | A-5 | D-154 |
| 408X | A-5 | D-155 |
| 409X | A-5 | D-156 |
| 410X | A-5 | D-157 |
| 411X | A-5 | D-158 |
| 412X | A-5 | D-159 |
| 413X | A-5 | D-160 |
| 414X | A-5 | D-161 |
| 415X | A-5 | D-162 |
| 416X | A-5 | D-163 |
| 417X | A-5 | D-164 |
| 418X | A-5 | D-165 |
| 419X | A-5 | D-166 |
| 420X | A-5 | D-167 |
| 421X | A-5 | D-168 |
| 422X | A-5 | D-169 |
| 423X | A-5 | D-170 |
| 424X | A-5 | D-171 |
| 425X | A-5 | D-172 |
| 426X | A-5 | D-173 |
| 427X | A-5 | D-174 |
| 428X | A-5 | D-175 |
| 429X | A-5 | D-176 |
| 430X | A-5 | D-177 |
| 431X | A-5 | D-178 |
| 432X | A-5 | D-179 |
| 433X | A-5 | D-180 |
| 434X | A-5 | D-181 |
| 435X | A-5 | D-182 |
| 436X | A-5 | D-183 |
| 437X | A-5 | D-184 |
| 438X | A-5 | D-185 |
| 439X | A-5 | D-186 |
| 440X | A-5 | D-187 |
| 441X | A-5 | D-188 |
| 442X | A-5 | D-189 |
| 443X | A-5 | D-190 |
| 444X | A-5 | D-191 |
| 445X | A-5 | D-192 |
| 446X | A-5 | D-193 |
| 447X | A-5 | D-194 |
| 448X | A-5 | D-195 |
| 449X | A-5 | D-196 |
| 450X | A-5 | D-197 |
| 451X | A-5 | D-198 |
| 452X | A-5 | D-199 |
| 453X | A-5 | D-200 |
| 454X | A-5 | D-201 |
| 455X | A-5 | D-202 |
| 456X | A-5 | D-203 |
| 457X | A-5 | D-204 |
| 458X | A-5 | D-205 |
| 459X | A-5 | D-206 |
| 460X | A-5 | D-207 |
| 461X | A-5 | D-208 |
| 462X | A-5 | D-209 |
| 463X | A-5 | D-210 |
| 464X | A-5 | D-211 |
| 465X | A-5 | D-212 |
| 466X | A-5 | D-213 |
| 467X | A-5 | D-214 |
| 468X | A-5 | D-215 |
| 469X | A-5 | D-216 |
| 470X | A-5 | D-217 |
| 471X | A-5 | D-218 |
| 472X | A-5 | D-219 |
| 473X | A-5 | D-220 |
| 474X | A-5 | D-221 |
| 475X | A-5 | D-222 |
| 476X | A-5 | D-223 |
| 477X | A-5 | D-224 |
| 478X | A-5 | D-225 |
| 479X | A-5 | D-226 |
| 480X | A-5 | D-227 |
| 481X | A-5 | D-228 |
| 482X | A-5 | D-229 |
| 483X | A-5 | D-230 |
| 484X | A-5 | D-231 |
| 485X | A-5 | D-232 |
| 486X | A-7 | D-1 |
| 487X | A-7 | D-2 |
| 488X | A-7 | D-3 |
| 489X | A-7 | D-4 |
| 490X | A-7 | D-5 |
| 491X | A-7 | D-6 |
| 492X | A-7 | D-7 |
| 493X | A-7 | D-8 |
| 494X | A-7 | D-9 |
| 495X | A-7 | D-10 |
| 496X | A-7 | D-11 |
| 497X | A-7 | D-12 |
| 498X | A-7 | D-13 |
| 499X | A-7 | D-14 |
| 500X | A-7 | D-15 |
| 501X | A-7 | D-16 |
| 502X | A-7 | D-17 |
| 503X | A-7 | D-18 |
| 504X | A-7 | D-19 |
| 505X | A-7 | D-20 |
| 506X | A-7 | D-21 |
| 507X | A-7 | D-22 |
| 508X | A-7 | D-23 |
| 509X | A-7 | D-24 |
| 510X | A-7 | D-25 |
| 511X | A-7 | D-26 |
| 512X | A-7 | D-27 |
| 513X | A-7 | D-28 |
| 514X | A-7 | D-29 |
| 515X | A-7 | D-30 |
| 516X | A-7 | D-31 |
| 517X | A-7 | D-32 |
| 518X | A-7 | D-33 |
| 519X | A-7 | D-34 |
| 520X | A-7 | D-35 |
| 521X | A-7 | D-36 |
| 522X | A-7 | D-37 |
| 523X | A-7 | D-38 |
| 524X | A-7 | D-39 |
| 525X | A-7 | D-40 |
| 526X | A-7 | D-41 |
| 527X | A-7 | D-42 |
| 528X | A-7 | D-43 |
| 529X | A-7 | D-44 |
| 530X | A-7 | D-45 |
| 531X | A-7 | D-46 |
| 532X | A-7 | D-47 |
| 533X | A-7 | D-48 |
| 534X | A-7 | D-49 |
| 535X | A-7 | D-50 |
| 536X | A-7 | D-51 |
| 537X | A-7 | D-52 |
| 538X | A-7 | D-53 |
| 539X | A-7 | D-54 |
| 540X | A-7 | D-55 |
| 541X | A-7 | D-56 |
| 542X | A-7 | D-57 |
| 543X | A-7 | D-58 |
| 544X | A-7 | D-59 |
| 545X | A-7 | D-60 |
| 546X | A-7 | D-61 |
| 547X | A-7 | D-62 |
| 548X | A-7 | D-63 |
| 549X | A-7 | D-64 |
| 550X | A-7 | D-65 |
| 551X | A-7 | D-66 |
| 552X | A-7 | D-67 |
| 553X | A-7 | D-68 |
| 554X | A-7 | D-69 |
| 555X | A-7 | D-70 |
| 556X | A-7 | D-71 |
| 557X | A-7 | D-72 |
| 558X | A-7 | D-73 |
| 559X | A-7 | D-74 |
| 560X | A-7 | D-75 |
| 561X | A-7 | D-76 |
| 562X | A-7 | D-77 |
| 563X | A-7 | D-78 |
| 564X | A-7 | D-79 |
| 565X | A-7 | D-80 |
| 566X | A-7 | D-81 |
| 567X | A-7 | D-82 |
| 568X | A-7 | D-83 |
| 569X | A-7 | D-84 |
| 570X | A-7 | D-85 |
| 571X | A-7 | D-86 |
| 572X | A-7 | D-87 |
| 573X | A-7 | D-88 |
| 574X | A-7 | D-89 |
| 575X | A-7 | D-90 |
| 576X | A-7 | D-91 |
| 577X | A-7 | D-92 |
| 578X | A-7 | D-93 |
| 579X | A-7 | D-94 |
| 580X | A-7 | D-95 |
| 581X | A-7 | D-96 |
| 582X | A-7 | D-97 |
| 583X | A-7 | D-98 |
| 584X | A-7 | D-99 |
| 585X | A-7 | D-100 |
| 586X | A-7 | D-101 |
| 587X | A-7 | D-102 |
| 588X | A-7 | D-103 |
| 589X | A-7 | D-104 |
| 590X | A-7 | D-105 |
| 591X | A-7 | D-106 |
| 592X | A-7 | D-107 |
| 593X | A-7 | D-108 |
| 594X | A-7 | D-109 |
| 595X | A-7 | D-110 |
| 596X | A-7 | D-111 |
| 597X | A-7 | D-112 |
| 598X | A-7 | D-113 |
| 599X | A-7 | D-114 |
| 600X | A-7 | D-115 |
| 601X | A-7 | D-116 |
| 602X | A-7 | D-117 |
| 603X | A-7 | D-118 |
| 604X | A-7 | D-119 |
| 605X | A-7 | D-120 |
| 606X | A-7 | D-121 |
| 607X | A-7 | D-122 |
| 608X | A-7 | D-123 |
| 609X | A-7 | D-124 |
| 610X | A-7 | D-125 |
| 611X | A-7 | D-126 |
| 612X | A-7 | D-127 |
| 613X | A-7 | D-128 |
| 614X | A-7 | D-129 |
| 615X | A-7 | D-130 |
| 616X | A-7 | D-131 |
| 617X | A-7 | D-132 |
| 618X | A-7 | D-133 |
| 619X | A-7 | D-134 |
| 620X | A-7 | D-135 |
| 621X | A-7 | D-136 |
| 622X | A-7 | D-137 |
| 623X | A-7 | D-138 |
| 624X | A-7 | D-139 |
| 625X | A-7 | D-140 |
| 626X | A-7 | D-141 |
| 627X | A-7 | D-142 |
| 628X | A-7 | D-143 |
| 629X | A-7 | D-144 |
| 630X | A-7 | D-145 |
| 631X | A-7 | D-146 |
| 632X | A-7 | D-147 |
| 633X | A-7 | D-148 |
| 634X | A-7 | D-149 |
| 635X | A-7 | D-150 |
| 636X | A-7 | D-151 |
| 637X | A-7 | D-152 |
| 638X | A-7 | D-153 |
| 639X | A-7 | D-154 |
| 640X | A-7 | D-155 |
| 641X | A-7 | D-156 |
| 642X | A-7 | D-157 |
| 643X | A-7 | D-158 |
| 644X | A-7 | D-159 |
| 645X | A-7 | D-160 |
| 646X | A-7 | D-161 |
| 647X | A-7 | D-162 |
| 648X | A-7 | D-163 |
| 649X | A-7 | D-164 |
| 650X | A-7 | D-165 |
| 651X | A-7 | D-166 |
| 652X | A-7 | D-167 |
| 653X | A-7 | D-168 |
| 654X | A-7 | D-169 |
| 655X | A-7 | D-170 |
| 656X | A-7 | D-171 |
| 657X | A-7 | D-172 |
| 658X | A-7 | D-173 |
| 659X | A-7 | D-174 |
| 660X | A-7 | D-175 |
| 661X | A-7 | D-176 |
| 662X | A-7 | D-177 |
| 663X | A-7 | D-178 |
| 664X | A-7 | D-179 |
| 665X | A-7 | D-180 |
| 666X | A-7 | D-181 |
| 667X | A-7 | D-182 |
| 668X | A-7 | D-183 |
| 669X | A-7 | D-184 |
| 670X | A-7 | D-185 |
| 671X | A-7 | D-186 |
| 672X | A-7 | D-187 |
| 673X | A-7 | D-188 |
| 674X | A-7 | D-189 |
| 675X | A-7 | D-190 |
| 676X | A-7 | D-191 |
| 677X | A-7 | D-192 |
| 678X | A-7 | D-193 |
| 679X | A-7 | D-194 |
| 680X | A-7 | D-195 |
| 681X | A-7 | D-196 |
| 682X | A-7 | D-197 |
| 683X | A-7 | D-198 |
| 684X | A-7 | D-199 |
| 685X | A-7 | D-200 |
| 686X | A-7 | D-201 |
| 687X | A-7 | D-202 |
| 688X | A-7 | D-203 |
| 689X | A-7 | D-204 |
| 690X | A-7 | D-205 |
| 691X | A-7 | D-206 |
| 692X | A-7 | D-207 |
| 693X | A-7 | D-208 |
| 694X | A-7 | D-209 |
| 695X | A-7 | D-210 |
| 696X | A-7 | D-211 |
| 697X | A-7 | D-212 |
| 698X | A-7 | D-213 |
| 699X | A-7 | D-214 |
| 700X | A-7 | D-215 |
| 701X | A-7 | D-216 |
| 702X | A-7 | D-217 |
| 703X | A-7 | D-218 |
| 704X | A-7 | D-219 |
| 705X | A-7 | D-220 |
| 706X | A-7 | D-221 |
| 707X | A-7 | D-222 |
| 708X | A-7 | D-223 |
| 709X | A-7 | D-224 |
| 710X | A-7 | D-225 |
| 711X | A-7 | D-226 |
| 712X | A-7 | D-227 |
| 713X | A-7 | D-228 |
| 714X | A-7 | D-229 |
| 715X | A-7 | D-230 |
| 716X | A-7 | D-231 |
| 717X | A-7 | D-232 |

Tables X-4, X-4A and X-4B illustrate, by way of example and not limitation, some further combinations of the present invention wherein the combination comprises an amount of an ASBT inhibitor (Component 1), an amount of a cyclooxygenase-2 selective inhibitor (Component 2) and an amount of an HMG-CoA inhibitor (Component 3), wherein the amount of the ASBT inhibitor, the amount of the cyclooxygenase-2 selective inhibitor and the amount of the HMG-CoA inhibitor together constitute a hypercholesterolemia-related condition effective amount or an inflammation-related condition effective amount of the ASBT inhibitor and the cyclooxygenase-2 selective inhibitor and the HMG-CoA inhibitor.

TABLE X-4

| Example | Component | Component | Component |
|---|---|---|---|
| Number | 1 | 2 | 3 |
| 1y | A-3 | B-18 | C-1 |
| 2y | A-3 | B-19 | C-1 |
| 3y | A-3 | B-20 | C-1 |
| 4y | A-3 | B-21 | C-1 |
| 5y | A-3 | B-22 | C-1 |
| 6y | A-3 | B-23 | C-1 |
| 7y | A-3 | B-24 | C-1 |
| 8y | A-5 | B-18 | C-1 |
| 9y | A-5 | B-19 | C-1 |
| 10y | A-5 | B-20 | C-1 |
| 11y | A-5 | B-21 | C-1 |
| 12y | A-5 | B-22 | C-1 |
| 13y | A-5 | B-23 | C-1 |
| 14y | A-5 | B-24 | C-1 |
| 15y | A-7 | B-18 | C-1 |
| 16y | A-7 | B-19 | C-1 |
| 17y | A-7 | B-20 | C-1 |
| 18y | A-7 | B-21 | C-1 |
| 19y | A-7 | B-22 | C-1 |
| 20y | A-7 | B-23 | C-1 |
| 21y | A-7 | B-24 | C-1 |
| 22y | A-3 | B-18 | C-2 |
| 23y | A-3 | B-19 | C-2 |
| 24y | A-3 | B-20 | C-2 |
| 25y | A-3 | B-21 | C-2 |
| 26y | A-3 | B-22 | C-2 |
| 27y | A-3 | B-23 | C-2 |
| 28y | A-3 | B-24 | C-2 |
| 29y | A-5 | B-18 | C-2 |
| 30y | A-5 | B-19 | C-2 |
| 31y | A-5 | B-20 | C-2 |
| 32y | A-5 | B-21 | C-2 |
| 33y | A-5 | B-22 | C-2 |
| 34y | A-5 | B-23 | C-2 |
| 35y | A-5 | B-24 | C-2 |
| 36y | A-7 | B-18 | C-2 |
| 37y | A-7 | B-19 | C-2 |
| 38y | A-7 | B-20 | C-2 |
| 39y | A-7 | B-21 | C-2 |
| 40y | A-7 | B-22 | C-2 |
| 41y | A-7 | B-23 | C-2 |
| 42y | A-7 | B-24 | C-2 |
| 43y | A-7 | B-18 | C-3 |
| 44y | A-3 | B-19 | C-3 |
| 45y | A-3 | B-20 | C-3 |
| 46y | A-3 | B-21 | C-3 |
| 47y | A-3 | B-22 | C-3 |
| 48y | A-3 | B-23 | C-3 |
| 49y | A-3 | B-24 | C-3 |
| 50y | A-5 | B-18 | C-3 |
| 51y | A-5 | B-19 | C-3 |
| 52y | A-5 | B-20 | C-3 |
| 53y | A-5 | B-21 | C-3 |
| 54y | A-5 | B-22 | C-3 |
| 55y | A-5 | B-23 | C-3 |
| 56y | A-5 | B-24 | C-3 |
| 57y | A-7 | B-18 | C-3 |
| 58y | A-7 | B-19 | C-3 |
| 59y | A-7 | B-20 | C-3 |
| 60y | A-7 | B-21 | C-3 |
| 61y | A-7 | B-22 | C-3 |
| 62y | A-7 | B-23 | C-3 |
| 63y | A-7 | B-24 | C-3 |
| 64y | A-3 | B-18 | C-4 |
| 65y | A-3 | B-19 | C-4 |
| 66y | A-3 | B-20 | C-4 |
| 67y | A-3 | B-21 | C-4 |
| 68y | A-3 | B-22 | C-4 |
| 69y | A-3 | B-23 | C-4 |
| 70y | A-3 | B-24 | C-4 |
| 71y | A-5 | B-18 | C-4 |
| 72y | A-5 | B-19 | C-4 |
| 73y | A-5 | B-20 | C-4 |
| 74y | A-5 | B-21 | C-4 |
| 75y | A-5 | B-22 | C-4 |
| 76y | A-5 | B-23 | C-4 |
| 77y | A-5 | B-24 | C-4 |
| 78y | A-7 | B-18 | C-4 |
| 79y | A-7 | B-19 | C-4 |
| 80y | A-7 | B-20 | C-4 |
| 81y | A-7 | B-21 | C-4 |
| 82y | A-7 | B-22 | C-4 |
| 83y | A-7 | B-23 | C-4 |
| 84y | A-7 | B-24 | C-4 |
| 85y | A-3 | B-18 | C-5 |
| 86y | A-3 | B-19 | C-5 |
| 87y | A-3 | B-20 | C-5 |
| 88y | A-3 | B-21 | C-5 |
| 89y | A-3 | B-22 | C-5 |
| 90y | A-3 | B-23 | C-5 |
| 91y | A-3 | B-24 | C-5 |
| 92y | A-5 | B-18 | C-5 |
| 93y | A-5 | B-19 | C-5 |
| 94y | A-5 | B-20 | C-5 |
| 95y | A-5 | B-21 | C-5 |
| 96y | A-5 | B-22 | C-5 |
| 97y | A-5 | B-23 | C-5 |
| 98y | A-5 | B-24 | C-5 |
| 99y | A-7 | B-18 | C-5 |
| 100y | A-7 | B-19 | C-5 |
| 101y | A-7 | B-20 | C-5 |
| 102y | A-7 | B-21 | C-5 |
| 103y | A-7 | B-22 | C-5 |
| 104y | A-7 | B-23 | C-5 |
| 105y | A-7 | B-24 | C-5 |
| 106y | A-3 | B-18 | C-6 |
| 107y | A-3 | B-19 | C-6 |
| 108y | A-3 | B-20 | C-6 |
| 109y | A-3 | B-21 | C-6 |
| 110y | A-3 | B-22 | C-6 |
| 111y | A-3 | B-23 | C-6 |
| 112y | A-3 | B-24 | C-6 |
| 113y | A-5 | B-18 | C-6 |
| 114y | A-5 | B-19 | C-6 |
| 115y | A-5 | B-20 | C-6 |
| 116y | A-5 | B-21 | C-6 |
| 117y | A-5 | B-22 | C-6 |
| 118y | A-5 | B-23 | C-6 |
| 119y | A-5 | B-24 | C-6 |
| 120y | A-7 | B-18 | C-6 |
| 121y | A-7 | B-19 | C-6 |
| 122y | A-7 | B-20 | C-6 |
| 123y | A-7 | B-21 | C-6 |
| 124y | A-7 | B-22 | C-6 |
| 125y | A-7 | B-23 | C-6 |
| 126y | A-7 | B-24 | C-6 |
| 127y | A-3 | B-18 | C-7 |
| 128y | A-3 | B-19 | C-7 |
| 129y | A-3 | B-20 | C-7 |
| 130y | A-3 | B-21 | C-7 |
| 131y | A-3 | B-22 | C-7 |
| 132y | A-3 | B-23 | C-7 |
| 133y | A-3 | B-24 | C-7 |
| 134y | A-5 | B-18 | C-7 |
| 135y | A-5 | B-19 | C-7 |
| 136y | A-5 | B-20 | C-7 |
| 137y | A-5 | B-21 | C-7 |
| 138y | A-5 | B-22 | C-7 |
| 139y | A-5 | B-23 | C-7 |
| 140y | A-5 | B-24 | C-7 |
| 141y | A-7 | B-18 | C-7 |
| 142y | A-7 | B-19 | C-7 |
| 143y | A-7 | B-20 | C-7 |
| 144y | A-7 | B-21 | C-7 |
| 145y | A-7 | B-22 | C-7 |
| 146y | A-7 | B-23 | C-7 |
| 147y | A-7 | B-24 | C-7 |
| 148y | A-3 | B-18 | C-8 |
| 149y | A-3 | B-19 | C-8 |
| 150y | A-3 | B-20 | C-8 |
| 151y | A-3 | B-21 | C-8 |
| 152y | A-3 | B-22 | C-8 |
| 153y | A-3 | B-23 | C-8 |
| 154y | A-3 | B-24 | C-8 |
| 155y | A-5 | B-18 | C-8 |
| 156y | A-5 | B-19 | C-8 |
| 157y | A-5 | B-20 | C-8 |
| 158y | A-5 | B-21 | C-8 |
| 159y | A-5 | B-22 | C-8 |
| 160y | A-5 | B-23 | C-8 |
| 161y | A-5 | B-24 | C-8 |
| 162y | A-7 | B-18 | C-8 |
| 163y | A-7 | B-19 | C-8 |
| 164y | A-7 | B-20 | C-8 |
| 165y | A-7 | B-21 | C-8 |
| 166y | A-7 | B-22 | C-8 |
| 167y | A-7 | B-23 | C-8 |
| 168y | A-7 | B-24 | C-8 |
| 169y | A-3 | B-18 | C-9 |
| 170y | A-3 | B-19 | C-9 |
| 171y | A-3 | B-20 | C-9 |
| 172y | A-3 | B-21 | C-9 |
| 173y | A-3 | B-22 | C-9 |
| 174y | A-3 | B-23 | C-9 |
| 175y | A-3 | B-24 | C-9 |
| 176y | A-5 | B-18 | C-9 |
| 177y | A-5 | B-19 | C-9 |
| 178y | A-5 | B-20 | C-9 |
| 179y | A-5 | B-21 | C-9 |
| 180y | A-5 | B-22 | C-9 |
| 181y | A-5 | B-23 | C-9 |
| 182y | A-5 | B-24 | C-9 |
| 183y | A-7 | B-18 | C-9 |
| 184y | A-7 | B-19 | C-9 |
| 185y | A-7 | B-20 | C-9 |
| 186y | A-7 | B-21 | C-9 |
| 187y | A-7 | B-22 | C-9 |
| 188y | A-7 | B-23 | C-9 |
| 189y | A-7 | B-24 | C-9 |

TABLE X-4A

| Example | | | |
|---|---|---|---|
| Number | Component 1 | Component 2 | Component 3 |
| 190y | Any one or more of | D-1 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21, A-22 | | Table 8 |
| 191y | Any one or more of | D-2 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 192y | Any one or more of | D-3 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 193y | Any one or more of | D-4 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 194y | Any one or more of | D-5 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 195y | Any one or more of | D-6 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 196y | Any one or more of | D-7 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 197y | Any one or more of | D-8 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 198y | Any one or more of | D-9 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 199y | Any one or more of | D-10 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 200y | Any one or more of | D-11 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 201y | Any one or more of | D-12 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 202y | Any one or more of | D-13 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 203y | Any one or more of | D-14 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 204y | Any one or more of | D-15 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 205y | Any one or more of | D-16 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 206y | Any one or more of | D-17 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 207y | Any one or more of | D-18 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 208y | Any one or more of | D-19 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 209y | Any one or more of | D-20 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 210y | Any one or more of | D-21 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 211y | Any one or more of | D-22 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 212y | Any one or more of | D-23 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 213y | Any one or more of | D-24 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 214y | Any one or more of | D-25 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 215y | Any one or more of | D-26 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 216y | Any one or more of | D-27 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 217y | Any one or more of | D-28 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 218y | Any one or more of | D-29 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 219y | Any one or more of | D-30 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 220y | Any one or more of | D-31 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 221y | Any one or more of | D-32 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 222y | Any one or more of | D-33 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 223y | Any one or more of | D-34 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 224y | Any one or more of | D-35 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 225y | Any one or more of | D-36 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 226y | Any one or more of | D-37 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 227y | Any one or more of | D-38 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 228y | Any one or more of | D-39 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 229y | Any one or more of | D-40 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 230y | Any one or more of | D-41 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 231y | Any one or more of | D-42 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 232y | Any one or more of | D-43 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 233y | Any one or more of | D-44 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 234y | Any one or more of | D-45 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 235y | Any one or more of | D-46 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 236y | Any one or more of | D-47 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 237y | Any one or more of | D-48 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 238y | Any one or more of | D-49 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 239y | Any one or more of | D-50 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 240y | Any one or more of | D-51 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 241y | Any one or more of | D-52 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 242y | Any one or more of | D-53 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 243y | Any one or more of | D-54 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 244y | Any one or more of | D-55 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 245y | Any one or more of | D-56 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 246y | Any one or more of | D-57 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 247y | Any one or more of | D-58 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 248y | Any one or more of | D-59 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 249y | Any one or more of | D-60 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 250y | Any one or more of | D-61 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 251y | Any one or more of | D-62 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 252y | Any one or more of | D-63 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 253y | Any one or more of | D-64 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 254y | Any one or more of | D-65 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 255y | Any one or more of | D-66 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 256y | Any one or more of | D-67 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 257y | Any one or more of | D-68 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 258y | Any one or more of | D-69 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 259y | Any one or more of | D-70 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 260y | Any one or more of | D-71 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 261y | Any one or more of | D-72 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 262y | Any one or more of | D-73 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 263y | Any one or more of | D-74 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 264y | Any one or more of | D-75 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 265y | Any one or more of | D-76 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 266y | Any one or more of | D-77 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 267y | Any one or more of | D-78 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 268y | Any one or more of | D-79 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 269y | Any one or more of | D-80 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 270y | Any one or more of | D-81 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 271y | Any one or more of | D-82 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 272y | Any one or more of | D-83 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 273y | Any one or more of | D-84 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 274y | Any one or more of | D-85 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 275y | Any one or more of | D-86 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 276y | Any one or more of | D-87 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 277y | Any one or more of | D-88 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 278y | Any one or more of | D-89 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 279y | Any one or more of | D-90 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 280y | Any one or more of | D-91 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 281y | Any one or more of | D-92 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 282y | Any one or more of | D-93 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 283y | Any one or more of | D-94 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 284y | Any one or more of | D-95 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 285y | Any one or more of | D-96 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 286y | Any one or more of | D-97 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 287y | Any one or more of | D-98 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 288y | Any one or more of | D-99 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 289y | Any one or more of | D-100 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 290y | Any one or more of | D-101 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 291y | Any one or more of | D-102 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 292y | Any one or more of | D-103 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 293y | Any one or more of | D-104 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 294y | Any one or more of | D-105 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 295y | Any one or more of | D-106 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 296y | Any one or more of | D-107 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 297y | Any one or more of | D-108 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 298y | Any one or more of | D-109 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 299y | Any one or more of | D-110 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 300y | Any one or more of | D-111 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 301y | Any one or more of | D-112 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 302y | Any one or more of | D-113 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 303y | Any one or more of | D-114 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 304y | Any one or more of | D-115 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 305y | Any one or more of | D-116 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 306y | Any one or more of | D-117 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 307y | Any one or more of | D-118 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 308y | Any one or more of | D-119 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 309y | Any one or more of | D-120 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 310y | Any one or more of | D-121 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 311y | Any one or more of | D-122 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 312y | Any one or more of | D-123 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 313y | Any one or more of | D-124 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 314y | Any one or more of | D-125 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 315y | Any one or more of | D-126 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 316y | Any one or more of | D-127 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 317y | Any one or more of | D-128 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 318y | Any one or more of | D-129 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 319y | Any one or more of | D-130 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 320y | Any one or more of | D-131 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 321y | Any one or more of | D-132 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 322y | Any one or more of | D-133 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 323y | Any one or more of | D-134 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 324y | Any one or more of | D-135 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 325y | Any one or more of | D-136 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 326y | Any one or more of | D-137 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 327y | Any one or more of | D-138 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 328y | Any one or more of | D-139 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 329y | Any one or more of | D-140 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 330y | Any one or more of | D-141 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 331y | Any one or more of | D-142 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 332y | Any one or more of | D-143 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 333y | Any one or more of | D-144 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 334y | Any one or more of | D-145 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 335y | Any one or more of | D-146 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 336y | Any one or more of | D-147 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 337y | Any one or more of | D-148 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 338y | Any one or more of | D-149 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 339y | Any one or more of | D-150 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 340y | Any one or more of | D-151 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 341y | Any one or more of | D-152 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 342y | Any one or more of | D-153 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 343y | Any one or more of | D-154 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 344y | Any one or more of | D-155 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 345y | Any one or more of | D-156 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 346y | Any one or more of | D-157 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 347y | Any one or more of | D-158 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 348y | Any one or more of | D-159 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 349y | Any one or more of | D-160 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 350y | Any one or more of | D-161 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 351y | Any one or more of | D-162 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 352y | Any one or more of | D-163 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 353y | Any one or more of | D-164 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 354y | Any one or more of | D-165 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 355y | Any one or more of | D-166 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 356y | Any one or more of | D-167 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 357y | Any one or more of | D-168 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 358y | Any one or more of | D-169 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 359y | Any one or more of | D-170 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 360y | Any one or more of | D-171 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 361y | Any one or more of | D-172 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 362y | Any one or more of | D-173 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 363y | Any one or more of | D-174 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 364y | Any one or more of | D-175 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 365y | Any one or more of | D-176 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 366y | Any one or more of | D-177 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 367y | Any one or more of | D-178 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 368y | Any one or more of | D-179 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 369y | Any one or more of | D-180 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 370y | Any one or more of | D-181 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 371y | Any one or more of | D-182 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 372y | Any one or more of | D-183 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 373y | Any one or more of | D-184 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 374y | Any one or more of | D-185 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 375y | Any one or more of | D-186 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 376y | Any one or more of | D-187 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 377y | Any one or more of | D-188 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 378y | Any one or more of | D-189 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 379y | Any one or more of | D-190 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 380y | Any one or more of | D-191 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 381y | Any one or more of | D-192 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 382y | Any one or more of | D-193 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 383y | Any one or more of | D-194 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 384y | Any one or more of | D-195 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 385y | Any one or more of | D-196 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 386y | Any one or more of | D-197 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 387y | Any one or more of | D-198 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 388y | Any one or more of | D-199 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 389y | Any one or more of | D-200 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 390y | Any one or more of | D-201 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 391y | Any one or more of | D-202 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 392y | Any one or more of | D-203 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 393y | Any one or more of | D-204 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 394y | Any one or more of | D-205 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 395y | Any one or more of | D-206 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 396y | Any one or more of | D-207 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 397y | Any one or more of | D-208 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 398y | Any one or more of | D-209 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 399y | Any one or more of | D-210 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 400y | Any one or more of | D-211 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 401y | Any one or more of | D-212 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 402y | Any one or more of | D-213 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 403y | Any one or more of | D-214 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 404y | Any one or more of | D-215 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 405y | Any one or more of | D-216 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 406y | Any one or more of | D-217 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 407y | Any one or more of | D-218 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 408y | Any one or more of | D-219 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 409y | Any one or more of | D-220 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 410y | Any one or more of | D-221 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 411y | Any one or more of | D-222 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 412y | Any one or more of | D-223 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 413y | Any one or more of | D-224 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 414y | Any one or more of | D-225 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 415y | Any one or more of | D-226 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 416y | Any one or more of | D-227 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 417y | Any one or more of | D-228 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 418y | Any one or more of | D-229 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 419y | Any one or more of | D-230 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 420y | Any one or more of | D-231 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |
| 421y | Any one or more of | D-232 | Any one or more |
| | A-1, A-2, A-3, A- | | of C-1, C-2, C- |
| | 4, A-5, A-6, A-7, | | 3, C-4, C-5, C- |
| | A-8, A-9, A-10, A- | | 6, C-7, C-8, C- |
| | 11, A-12, A-13, A- | | 9, and HMG-CoA |
| | 14, A-15, A-16, A- | | Reductase |
| | 17, A-18, A-19, A- | | Inhibitors of |
| | 20, A-21 and A-22 | | Table 8 |

TABLE X-4B

| Example | | | |
|---|---|---|---|
| Number | Component 1 | Component 2 | Component 3 |
| 422y | Any one or more | D-1 to D-5 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21, | | |
| | A-22 | | |
| 423y | Any one or more | D-6 to D-10 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 424y | Any one or more | D-11 to D-15 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 425y | Any one or more | D-16 to D-20 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 426y | Any one or more | D-21 to D-25 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 427y | Any one or more | D-26 to D-30 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 428y | Any one or more | D-31 to D-35 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 429y | Any one or more | D-36 to D-40 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 430y | Any one or more | D-41 to D-45 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 431y | Any one or more | D-46 to D-50 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 432y | Any one or more | D-51 to D-55 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 433y | Any one or more | D-56 to D-60 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 434y | Any one or more | D-61 to D-65 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 435y | Any one or more | D-66 to D-70 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 436y | Any one or more | D-71 to D-75 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 437y | Any one or more | D-76 to D-80 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 438y | Any one or more | D-81 to D-85 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 439y | Any one or more | D-86 to D-90 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 440y | Any one or more | D-91 to D-95 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 441y | Any one or more | D-96 to D-100 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 442y | Any one or more | D-101 to D-105 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 443y | Any one or more | D-106 to D-110 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 444y | Any one or more | D-111 to D-115 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 445y | Any one or more | D-116 to D-120 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 446y | Any one or more | D-121 to D-125 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 447y | Any one or more | D-126 to D-130 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 448y | Any one or more | D-131 to D-135 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 449y | Any one or more | D-136 to D-140 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 450y | Any one or more | D-141 to D-145 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 451y | Any one or more | D-146 to D-150 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 452y | Any one or more | D-151 to D-155 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 453y | Any one or more | D-156 to D-160 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 454y | Any one or more | D-161 to D-165 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 455y | Any one or more | D-166 to D-170 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 456y | Any one or more | D-171 to D-175 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 457y | Any one or more | D-176 to D-180 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 458y | Any one or more | D-181 to D-185 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 459y | Any one or more | D-186 to D-190 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 460y | Any one or more | D-191 to D-195 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 461y | Any one or more | D-196 to D-200 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 462y | Any one or more | D-201 to D-205 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 463y | Any one or more | D-206 to D-210 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 464y | Any one or more | D-211 to D-215 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 465y | Any one or more | D-216 to D-220 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 466y | Any one or more | D-221 to D-225 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 467y | Any one or more | D-226 to D-230 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |
| 468y | Any one or more | D-231 to D-232 | Any one or more |
| | of A-1, A-2, A- | | of C-1, C-2, C- |
| | 3, A-4, A-5, A- | | 3, C-4, C-5, C- |
| | 6, A-7, A-8, A- | | 6, C-7, C-8, C- |
| | 9, A-10, A-11, | | 9, and HMG-CoA |
| | A-12, A-13, A- | | Reductase |
| | 14, A-15, A-16, | | Inhibitors of |
| | A-17, A-18, A- | | Table 8 |
| | 19, A-20, A-21 | | |
| | and A-22 | | |

Table X-5 illustrates, by way of example and not limitation, some of the many combinations of the present invention wherein the combination comprises an amount of an HMG Co-A reductase inhibitor (Component 1) and an amount of a chromene cyclooxygenase inhibitor (Component 2), wherein the amount of the HMG Co-A reductase inhibitor and the amount of the chromene cyclooxygenase inhibitor together constitute a hypercholesterolemia-related condition effective amount or an inflammation-related condition effective amount of the HMG Co-A reductase inhibitor and the chromene cyclooxygenase inhibitor.

TABLE X-5

| Example | | |
|---|---|---|
| Number | Component 1 | Component 2 |
| 1z | Benfluorex | B-3 |
| 2z | Benfluorex | B-4 |
| 3z | Benfluorex | B-5 |
| 4z | Benfluorex | B-6 |
| 5z | Benfluorex | B-7 |
| 6z | Benfluorex | B-8 |
| 7z | Benfluorex | B-9 |
| 8z | Benfluorex | B-10 |
| 9z | Benfluorex | B-11 |
| 10z | Benfluorex | B-12 |
| 11z | Benfluorex | B-13 |
| 12z | Benfluorex | B-14 |
| 13z | Benfluorex | B-15 |
| 14z | Benfluorex | B-16 |
| 15z | Benfluorex | B-17 |
| 16z | Fluvastatin | B-3 |
| 17z | Fluvastatin | B-4 |
| 18z | Fluvastatin | B-5 |
| 19z | Fluvastatin | B-6 |
| 20z | Fluvastatin | B-7 |
| 21z | Fluvastatin | B-8 |
| 22z | Fluvastatin | B-9 |
| 23z | Fluvastatin | B-10 |
| 24z | Fluvastatin | B-11 |
| 25z | Fluvastatin | B-12 |
| 26z | Fluvastatin | B-13 |
| 27z | Fluvastatin | B-14 |
| 28z | Fluvastatin | B-15 |
| 29z | Fluvastatin | B-16 |
| 30z | Fluvastatin | B-17 |
| 31z | Lovastatin | B-3 |
| 32z | Lovastatin | B-4 |
| 33z | Lovastatin | B-5 |
| 34z | Lovastatin | B-6 |
| 35z | Lovastatin | B-7 |
| 36z | Lovastatin | B-8 |
| 37z | Lovastatin | B-9 |
| 38z | Lovastatin | B-10 |
| 39z | Lovastatin | B-11 |
| 40z | Lovastatin | B-12 |
| 41z | Lovastatin | B-13 |
| 42z | Lovastatin | B-14 |
| 43z | Lovastatin | B-15 |
| 44z | Lovastatin | B-16 |
| 45z | Lovastatin | B-17 |
| 46z | Pravastatin | B-3 |
| 47z | Pravastatin | B-4 |
| 48z | Pravastatin | B-5 |
| 49z | Pravastatin | B-6 |
| 50z | Pravastatin | B-7 |
| 51z | Pravastatin | B-8 |
| 52z | Pravastatin | B-9 |
| 53z | Pravastatin | B-10 |
| 54z | Pravastatin | B-11 |
| 55z | Pravastatin | B-12 |
| 56z | Pravastatin | B-13 |
| 57z | Pravastatin | B-14 |
| 58z | Pravastatin | B-15 |
| 59z | Pravastatin | B-16 |
| 60z | Pravastatin | B-17 |
| 61z | Simvastatin | B-3 |
| 62z | Simvastatin | B-4 |
| 63z | Simvastatin | B-5 |
| 64z | Simvastatin | B-6 |
| 65z | Simvastatin | B-7 |
| 66z | Simvastatin | B-8 |
| 67z | Simvastatin | B-9 |
| 68z | Simvastatin | B-10 |
| 69z | Simvastatin | B-11 |
| 70z | Simvastatin | B-12 |
| 71z | Simvastatin | B-13 |
| 72z | Simvastatin | B-14 |
| 73z | Simvastatin | B-15 |
| 74z | Simvastatin | B-16 |
| 75z | Simvastatin | B-17 |
| 76z | Atorvastatin | B-3 |
| 77z | Atorvastatin | B-4 |
| 78z | Atorvastatin | B-5 |
| 79z | Atorvastatin | B-6 |
| 80z | Atorvastatin | B-7 |
| 81z | Atorvastatin | B-8 |
| 82z | Atorvastatin | B-9 |
| 83z | Atorvastatin | B-10 |
| 84z | Atorvastatin | B-11 |
| 85z | Atorvastatin | B-12 |
| 86z | Atorvastatin | B-13 |
| 87z | Atorvastatin | B-14 |
| 88z | Atorvastatin | B-15 |
| 89z | Atorvastatin | B-16 |
| 90z | Atorvastatin | B-17 |
| 91z | Cerivastatin | B-3 |
| 92z | Cerivastatin | B-4 |
| 93z | Cerivastatin | B-5 |
| 94z | Cerivastatin | B-6 |
| 95z | Cerivastatin | B-7 |
| 96z | Cerivastatin | B-8 |
| 97z | Cerivastatin | B-9 |
| 98z | Cerivastatin | B-10 |
| 99z | Cerivastatin | B-11 |
| 100z | Cerivastatin | B-12 |
| 101z | Cerivastatin | B-13 |
| 102z | Cerivastatin | B-14 |
| 103z | Cerivastatin | B-15 |
| 104z | Cerivastatin | B-16 |
| 105z | Cerivastatin | B-17 |
| 106z | Vervastatin | B-3 |
| 107z | Vervastatin | B-4 |
| 108z | Vervastatin | B-5 |
| 109z | Vervastatin | B-6 |
| 110z | Vervastatin | B-7 |
| 111z | Vervastatin | B-8 |
| 112z | Vervastatin | B-9 |
| 113z | Vervastatin | B-10 |
| 114z | Vervastatin | B-11 |
| 115z | Vervastatin | B-12 |
| 116z | Vervastatin | B-13 |
| 117z | Vervastatin | B-14 |
| 118z | Vervastatin | B-15 |
| 119z | Vervastatin | B-16 |
| 120z | Vervastatin | B-17 |
| 121z | Rosuvastatin | B-3 |
| | (ZD-4522) | |
| 122z | Rosuvastatin | B-4 |
| | (ZD-4522) | |
| 123z | Rosuvastatin | B-5 |
| | (ZD-4522) | |
| 124z | Rosuvastatin | B-6 |
| | (ZD-4522) | |
| 125z | Rosuvastatin | B-7 |
| | (ZD-4522) | |
| 126z | Rosuvastatin | B-8 |
| | (ZD-4522) | |
| 127z | Rosuvastatin | B-9 |
| | (ZD-4522) | |
| 128z | Rosuvastatin | B-10 |
| | (ZD-4522) | |
| 129z | Rosuvastatin | B-11 |
| | (ZD-4522) | |
| 130z | Rosuvastatin | B-12 |
| | (ZD-4522) | |
| 131z | Rosuvastatin | B-13 |
| | (ZD-4522) | |
| 132z | Rosuvastatin | B-14 |
| | (ZD-4522) | |
| 133z | Rosuvastatin | B-15 |
| | (ZD-4522) | |
| 134z | Rosuvastatin | B-16 |
| | (ZD-4522) | |
| 135z | Rosuvastatin | B-17 |
| | (ZD-4522) | |
| 136z | Itavastatin | B-3 |
| 137z | Itavastatin | B-4 |
| 138z | Itavastatin | B-5 |
| 139z | Itavastatin | B-6 |
| 140z | Itavastatin | B-7 |
| 141z | Itavastatin | B-8 |
| 142z | Itavastatin | B-9 |
| 143z | Itavastatin | B-10 |
| 144z | Itavastatin | B-11 |
| 145z | Itavastatin | B-12 |
| 146z | Itavastatin | B-13 |
| 147z | Itavastatin | B-14 |
| 148z | Itavastatin | B-15 |
| 149z | Itavastatin | B-16 |
| 150z | Itavastatin | B-17 |
| 151z | Delvastatin | B-3 |
| 152z | Delvastatin | B-4 |
| 153z | Delvastatin | B-5 |
| 154z | Delvastatin | B-6 |
| 155z | Delvastatin | B-7 |
| 156z | Delvastatin | B-8 |
| 157z | Delvastatin | B-9 |
| 158z | Delvastatin | B-10 |
| 159z | Delvastatin | B-11 |
| 160z | Delvastatin | B-12 |
| 161z | Delvastatin | B-13 |
| 162z | Delvastatin | B-14 |
| 163z | Delvastatin | B-15 |
| 164z | Delvastatin | B-16 |
| 165z | Delvastatin | B-17 |
| 166z | Mevastatin | B-3 |
| 167z | Mevastatin | B-4 |
| 168z | Mevastatin | B-5 |
| 169z | Mevastatin | B-6 |
| 170z | Mevastatin | B-7 |
| 171z | Mevastatin | B-8 |
| 172z | Mevastatin | B-9 |
| 173z | Mevastatin | B-10 |
| 174z | Mevastatin | B-11 |
| 175z | Mevastatin | B-12 |
| 176z | Mevastatin | B-13 |
| 177z | Mevastatin | B-14 |
| 178z | Mevastatin | B-15 |
| 179z | Mevastatin | B-16 |
| 180z | Mevastatin | B-17 |

TableS X-5A and X-5B illustrate, by way of example and not limitation, some of the many combinations of the present invention wherein the combination comprises an amount of an HMG Co-A reductase inhibitor (Component 1) and an amount of a cyclooxygenase-2 selective inhibitor (Component 2), wherein the amount of the HMG Co-A reductase inhibitor and the amount of the cyclooxygenase-2 selective inhibitor tqgether constitute a hypercholesterolemia-related condition effective amount or an inflammation-related condition effective amount of the HMG Co-A reductase inhibitor and the cyclooxygenase-2 selective inhibitor.

TABLE 5A

| Example Number | Component 1 | Component 2 |
|---|---|---|
| 181z | Any one or more of | D-1 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 182z | Any one or more of | D-2 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 183z | Any one or more of | D-3 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 184z | Any one or more of | D-4 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 185z | Any one or more of | D-5 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 186z | Any one or more of | D-6 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 187z | Any one or more of | D-7 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 188z | Any one or more of | D-8 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 189z | Any one or more of | D-9 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 190z | Any one or more of | D-10 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 191z | Any one or more of | D-11 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 192z | Any one or more of | D-12 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 193z | Any one or more of | D-13 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 194z | Any one or more of | D-14 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 195z | Any one or more of | D-15 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 196z | Any one or more of | D-16 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 197z | Any one or more of | D-17 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 198z | Any one or more of | D-18 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 199z | Any one or more of | D-19 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 200z | Any one or more of | D-20 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 201z | Any one or more of | D-21 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 202z | Any one or more of | D-22 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 203z | Any one or more of | D-23 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 204z | Any one or more of | D-24 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 205z | Any one or more of | D-25 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 206z | Any one or more of | D-26 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 207z | Any one or more of | D-27 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 208z | Any one or more of | D-28 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 209z | Any one or more of | D-29 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 210z | Any one or more of | D-30 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 211z | Any one or more of | D-31 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 212z | Any one or more of | D-32 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 213z | Any one or more of | D-33 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 214z | Any one or more of | D-34 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 215z | Any one or more of | D-35 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 216z | Any one or more of | D-36 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 217z | Any one or more of | D-37 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 218z | Any one or more of | D-38 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 219z | Any one or more of | D-39 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 220z | Any one or more of | D-40 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 221z | Any one or more of | D-41 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 222z | Any one or more of | D-42 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 223z | Any one or more of | D-43 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 224z | Any one or more of | D-44 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 225z | Any one or more of | D-45 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 226z | Any one or more of | D-46 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 227z | Any one or more of | D-47 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 228z | Any one or more of | D-48 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 229z | Any one or more of | D-49 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 230z | Any one or more of | D-50 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 231z | Any one or more of | D-51 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 232z | Any one or more of | D-52 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 233z | Any one or more of | D-53 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 234z | Any one or more of | D-54 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 235z | Any one or more of | D-55 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 236z | Any one or more of | D-56 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 237z | Any one or more of | D-57 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 238z | Any one or more of | D-58 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 239z | Any one or more of | D-59 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 240z | Any one or more of | D-60 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 241z | Any one or more of | D-61 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 242z | Any one or more of | D-62 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 243z | Any one or more of | D-63 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 244z | Any one or more of | D-64 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 245z | Any one or more of | D-65 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 246z | Any one or more of | D-66 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 247z | Any one or more of | D-67 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 248z | Any one or more of | D-68 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 249z | Any one or more of | D-69 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 250z | Any one or more of | D-70 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 251z | Any one or more of | D-71 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 252z | Any one or more of | D-72 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 253z | Any one or more of | D-73 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 254z | Any one or more of | D-74 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 255z | Any one or more of | D-75 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 256z | Any one or more of | D-76 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 257z | Any one or more of | D-77 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 258z | Any one or more of | D-78 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 259z | Any one or more of | D-79 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 260z | Any one or more of | D-80 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 261z | Any one or more of | D-81 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 262z | Any one or more of | D-82 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 263z | Any one or more of | D-83 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 264z | Any one or more of | D-84 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 265z | Any one or more of | D-85 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 266z | Any one or more of | D-86 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 267z | Any one or more of | D-87 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 268z | Any one or more of | D-88 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 269z | Any one or more of | D-89 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 270z | Any one or more of | D-90 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 271z | Any one or more of | D-91 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 272z | Any one or more of | D-92 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 273z | Any one or more of | D-93 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 274z | Any one or more of | D-94 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 275z | Any one or more of | D-95 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 276z | Any one or more of | D-96 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 277z | Any one or more of | D-97 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 278z | Any one or more of | D-98 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 279z | Any one or more of | D-99 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 280z | Any one or more of | D-100 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 281z | Any one or more of | D-101 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 282z | Any one or more of | D-102 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 283z | Any one or more of | D-103 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 284z | Any one or more of | D-104 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 285z | Any one or more of | D-105 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 286z | Any one or more of | D-106 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 287z | Any one or more of | D-107 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 288z | Any one or more of | D-108 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 289z | Any one or more of | D-109 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 290z | Any one or more of | D-110 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 291z | Any one or more of | D-111 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 292z | Any one or more of | D-112 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 293z | Any one or more of | D-113 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 294z | Any one or more of | D-114 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 295z | Any one or more of | D-115 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 296z | Any one or more of | D-116 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 297z | Any one or more of | D-117 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 298z | Any one or more of | D-118 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 299z | Any one or more of | D-119 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 300z | Any one or more of | D-120 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 301z | Any one or more of | D-121 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 302z | Any one or more of | D-122 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 303z | Any one or more of | D-123 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 304z | Any one or more of | D-124 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 305z | Any one or more of | D-125 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 306z | Any one or more of | D-126 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 307z | Any one or more of | D-127 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 308z | Any one or more of | D-128 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 309z | Any one or more of | D-129 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 310z | Any one or more of | D-130 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 311z | Any one or more of | D-131 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 312z | Any one or more of | D-132 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 313z | Any one or more of | D-133 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 314z | Any one or more of | D-134 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 315z | Any one or more of | D-135 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 316z | Any one or more of | D-136 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 317z | Any one or more of | D-137 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 318z | Any one or more of | D-138 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 319z | Any one or more of | D-139 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 320z | Any one or more of | D-140 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 321z | Any one or more of | D-141 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 322z | Any one or more of | D-142 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 323z | Any one or more of | D-143 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 324z | Any one or more of | D-144 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 325z | Any one or more of | D-145 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 326z | Any one or more of | D-146 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 327z | Any one or more of | D-147 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 328z | Any one or more of | D-148 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 329z | Any one or more of | D-149 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 330z | Any one or more of | D-150 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 331z | Any one or more of | D-151 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 332z | Any one or more of | D-152 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 333z | Any one or more of | D-153 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 334z | Any one or more of | D-154 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 335z | Any one or more of | D-155 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 336z | Any one or more of | D-156 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 337z | Any one or more of | D-157 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 338z | Any one or more of | D-158 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 339z | Any one or more of | D-159 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 340z | Any one or more of | D-160 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 341z | Any one or more of | D-161 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 342z | Any one or more of | D-162 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 343z | Any one or more of | D-163 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 344z | Any one or more of | D-164 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 345z | Any one or more of | D-165 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 346z | Any one or more of | D-166 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 347z | Any one or more of | D-167 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 348z | Any one or more of | D-168 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 349z | Any one or more of | D-169 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 350z | Any one or more of | D-170 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 351z | Any one or more of | D-171 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 352z | Any one or more of | D-172 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 353z | Any one or more of | D-173 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 354z | Any one or more of | D-174 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 355z | Any one or more of | D-175 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 356z | Any one or more of | D-176 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 357z | Any one or more of | D-177 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 358z | Any one or more of | D-178 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 359z | Any one or more of | D-179 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 360z | Any one or more of | D-180 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 361z | Any one or more of | D-181 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 362z | Any one or more of | D-182 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 363z | Any one or more of | D-183 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 364z | Any one or more of | D-184 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 365z | Any one or more of | D-185 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 366z | Any one or more of | D-186 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 367z | Any one or more of | D-187 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 368z | Any one or more of | D-188 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 369z | Any one or more of | D-189 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 370z | Any one or more of | D-190 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 371z | Any one or more of | D-191 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 372z | Any one or more of | D-192 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 373z | Any one or more of | D-193 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 374z | Any one or more of | D-194 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 375z | Any one or more of | D-195 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 376z | Any one or more of | D-196 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 377z | Any one or more of | D-197 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 378z | Any one or more of | D-198 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 379z | Any one or more of | D-199 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 380z | Any one or more of | D-200 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 381z | Any one or more of | D-201 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 382z | Any one or more of | D-202 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 383z | Any one or more of | D-203 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 384z | Any one or more of | D-204 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 385z | Any one or more of | D-205 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 386z | Any one or more of | D-206 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 387z | Any one or more of | D-207 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 388z | Any one or more of | D-208 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 389z | Any one or more of | D-209 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 390z | Any one or more of | D-210 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 391z | Any one or more of | D-211 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 392z | Any one or more of | D-212 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 393z | Any one or more of | D-213 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 394z | Any one or more of | D-214 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 395z | Any one or more of | D-215 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 396z | Any one or more of | D-216 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 397z | Any one or more of | D-217 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 398z | Any one or more of | D-218 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 399z | Any one or more of | D-219 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 400z | Any one or more of | D-220 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 401z | Any one or more of | D-221 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 402z | Any one or more of | D-222 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 403z | Any one or more of | D-223 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 404z | Any one or more of | D-224 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 405z | Any one or more of | D-225 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 406z | Any one or more of | D-226 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 407z | Any one or more of | D-227 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 408z | Any one or more of | D-228 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 409z | Any one or more of | D-229 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 410z | Any one or more of | D-230 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 411z | Any one or more of | D-231 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 412z | Any one or more of | D-232 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |

TABLE 5B

| Example Number | Component 1 | Component 2 |
|---|---|---|
| 413z | Any one or more of | D-1 to D-5 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 414z | Any one or more of | D-6 to D-10 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 415z | Any one or more of | D-11 to D-15 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 416z | Any one or more of | D-16 to D-20 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 417z | Any one or more of | D-21 to D-25 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 418z | Any one or more of | D-26 to D30 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 419z | Any one or more of | D-31 to D-35 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 420z | Any one or more of | D-36 to D-40 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 421z | Any one or more of | D-41 to D-45 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 422z | Any one or more of | D-46 to D-50 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 423z | Any one or more of | D-51 to D-55 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 424z | Any one or more of | D-56 to D-60 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 425z | Any one or more of | D-61 to D-65 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 426z | Any one or more of | D-66 to D-70 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 427z | Any one or more of | D-71 to D-75 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 428z | Any one or more of | D-76 to D-80 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 429z | Any one or more of | D-81 to D-85 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 430z | Any one or more of | D-86 to D-90 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 431z | Any one or more of | D-91 to D-95 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 432z | Any one or more of | D-96 to D-100 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 433z | Any one or more of | D-101 to D-105 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 434z | Any one or more of | D-106 to D-110 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 435z | Any one or more of | D-111 to D-115 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 436z | Any one or more of | D-116 to D-120 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 437z | Any one or more of | D-121 to D-125 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 438z | Any one or more of | D-126 to D-130 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 439z | Any one or more of | D-131 to D-135 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 440z | Any one or more of | D-136 to D-140 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 441z | Any one or more of | D-141 to D-145 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 442z | Any one or more of | D-146 to D-150 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 443z | Any one or more of | D-151 to D-155 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 444z | Any one or more of | D-156 to D-160 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 445z | Any one or more of | D-161 to D-165 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 446z | Any one or more of | D-166 to D-170 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 447z | Any one or more of | D-171 to D-175 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 448z | Any one or more of | D-176 to D-180 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 449z | Any one or more of | D-181 to D-185 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 450z | Any one or more of | D-186 to D-190 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 451z | Any one or more of | D-191 to D-195 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 452z | Any one or more of | D-196 to D-200 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 453z | Any one or more of | D-201 to D-205 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 454z | Any one or more of | D-206 to D-210 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 455z | Any one or more of | D-211 to D-215 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 456z | Any one or more of | D-216 to D-220 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 457z | Any one or more of | D-221 to D-225 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 458z | Any one or more of | D-226 to D-230 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |
| 459z | Any one or more of | D-231 to D-232 |
| | Benfluorex, | |
| | Fluvastatin, | |
| | Lovastatin, | |
| | Pravastatin, | |
| | Simvastatin, | |
| | Atavastatin, | |
| | Cerivastatin, | |
| | Vervastatin, | |
| | Rosuvastatin, | |
| | Itavastatink | |
| | Delvastatin, and | |
| | Mevastatin | |

The above-noted combinations of: (1) ASBT inhibitor and COX-2 selective inhibitor (2) ASBT inhibitor, COX-2 selective inhibitor, and HMG Co-A reductase inhibitor, and (3) COX-2 selective inhibitor and HMG Co-A reductase inhibitor may independently be used to reduce total serum cholesterol in mammals including humans.

The above-noted combinations of: (1) ASBT inhibitor and COX-2 selective inhibitor and (2) ASBT inhibitor, COX-2 selective inhibitor, and HMG Co-A reductase inhibitor may independently be used to reduce serum thromboxane levels in mammals including humans.

The above-noted combinations of: (1) ASBT inhibitor and COX-2 selective inhibitor (2) ASBT inhibitor, COX-2 selective inhibitor, and HMG Co-A reductase inhibitor, and (3) COX-2 selective inhibitor and HMG Co-A reductase inhibitor may independently be used to reduce serum soluble intercellular cell adhesion molecule levels in mammals including humans.

The above-noted combinations of: (1) ASBT inhibitor and COX-2 selective inhibitor (2) ASBT inhibitor, COX-2 selective inhibitor, and HMG Co-A reductase inhibitor, and (3) COX-2 selective inhibitor and HMG Co-A reductase inhibitor may independently be used to reduce the T-cell content of an atherosclerotic lesion developing in mammals including humans.

The above-noted combinations of: (1) ASBT inhibitor and COX-2 selective inhibitor (2) ASBT inhibitor, COX-2 selective inhibitor, and HMG Co-A reductase inhibitor, and (3) COX-2 selective inhibitor and HMG Co-A reductase inhibitor may independently be used to increase smooth muscle cell content of an atherosclerotic lesion developing in the vasculature of mammals including humans.

The above-noted combinations of: (1) ASBT inhibitor and COX-2 selective inhibitor (2) ASBT inhibitor, COX-2 selective inhibitor, and HMG Co-A reductase inhibitor, and (3) COX-2 selective inhibitor and HMG Co-A reductase inhibitor may independently be used to reduce the aortic root atherosclerotic lesion area in mammals including humans.

The above-noted combinations of: (1) ASBT inhibitor and COX-2 selective inhibitor (2) ASBT inhibitor, COX-2 selective inhibitor, and HMG Co-A reductase inhibitor, and (3) COX-2 selective inhibitor and HMG Co-A reductase inhibitor may independently be used either as a treatment or as a prophylactic use in the treatment or prophylaxis of a hypercholesterolemia-related or an inflammation-related condition in a subject in need of such treatment or prevention.

Various Embodiments of the present invention are presented below for illustration.

### EMBODIMENTS

Various Embodiments are:

1. A method for treating or preventing a hypercholesterolemia-related or an inflammation-related condition in a subject in need of such treatment or prevention, comprising treating the subject with an amount of an apical sodium co-dependent bile acid transport inhibitor, an amount of a cyclooxygenase-2 selective inhibitor or prodrug, wherein the amount of the apical sodium co-dependent bile acid transport inhibitor, the amount of the cyclooxygenase-2 selective inhibitor together constitute a hypercholesterolemia-related condition effective amount or an inflammation-related condition effective amount of the apical sodium co-dependant bile acid transport inhibitor and the cyclooxygenase-2 selective inhibitor.

2. The method of Embodiment 1 wherein the amount of the apical sodium co-dependent bile acid transport inhibitor and the amount of the cyclooxygenase-2 selective inhibitor together constitute a hypercholesterolemia-related condition effective amount of the apical sodium co-dependent bile acid transport inhibitor and the cyclooxygenase inhibitor.

3. The method of Embodiment 1 wherein the amount of the apical sodium co-dependent bile acid transport inhibitor and the amount of the cyclooxygenase-2 selective inhibitor together constitute an inflammation-related condition effective amount of the apical sodium co-dependent bile acid transport inhibitor and the cyclooxygenase-2 selective inhibitor.

4. The method of Embodiment 1 wherein the condition is selected from the group consisting of gout, pancreatitis, cholelithiasis, biliary obstruction, ulcerative colitis, Crohn's disease, coronary artery disease, aneurysm, arteriosclerosis, atherosclerosis, myocardial infarction, embolism, stroke, thrombosis, angina, coronary plaque inflammation, bacterial-induced inflammation, viral induced inflammation, and inflammation wherein the inflammation is associated with a surgical procedure involving an artery, a vein or a capillary.

5. The method of Embodiment 4 wherein the condition is selected from the group consisting of coronary artery disease, atherosclerosis, and thrombosis.

6. The method of Embodiment 5 wherein the condition is coronary artery disease.

7. The method of Embodiment 1 wherein the cyclooxygenase-2 selective inhibitor is D-1, D-2, D-3, D-4, D-5, D-6, D-7, D-8, D-9, D-10, D-11, D-12, D-13, D-14, D-15, D-16, D-17, celecoxib (D-18), D-19, D-20, rofecoxib (D-21), D-22, D-23, D-24, D-25, D-26, D-27, D-28, D-29, D-30, D-31, D-32, D-33, D-34, D-35, D-36, D-37, D-38, D-39, D-40, D-41, D-42, D-43, D-44, D-45, D-46, D-47, D-48, D-49, D-50, D-51, D-52, D-53, D-54, D-55, D-56, D-57, D-58, D-59, D-60, D-61, D-62, D-63, D-64, D-65, D-66, D-67, D-68, D-69, D-70, D-71, D-72, D-73, D-74, D-75, D-76, D-77, D-78, D-79, D-80, D-81, D-82, D-83, D-84, D-85, D-86, D-87, D-88, D-89, D-90, D-91, D-92, D-93, D-94, D-95, D-96, D-97, D-98, D-99, D-100, D-101, D-102, D-103, D-104, D-105, D-106, D-107, D-108, D-109, D-110, D-111, D-112, D-113, D-114, D-115, D-116, D-117, D-118, D-119, D-120, D-121, D-122, D-123, D-124, D-125, D-126, D-127, D-128, D-129, D-130, D-131, D-132, D-133, D-134, D-135, D-136, D-137, D-138, D-139, D-140, D-141, D-142, D-143, D-144, D-145, D-146, D-147, D-148, D-149, D-150, D-151, D-152, D-153, D-154, D-155, D-156, D-157, D-158, D-159, D-160, D-161, D-162, D-163, D-164, D-165, D-166, D-167, D-168, D-169, D-170, D-171, D-172, D-173, D-174, D-175, D-176, D-177, D-178, D-179, D-180, D-181, D-182, D-183, D-184, D-185, D-186, D-187, D-188, D-189, D-190, D-191, D-192, D-193, D-194, D-195, D-196, D-197, D-198, D-199, D-200, D-201, D-202, D-203, D-204, D-205, D-206, D-207, D-208, D-209, D-210, D-211, D-212, D-213, D-214, D-215, D-216, D-217, D-218, D-219, D-220, D-221, D-222, D-223, D-224, D-225, D-226, D-227, D-228, D-229, D-230, D-231, D-232, or a pharmaceutically acceptable salt or derivative or prodrug thereof.

8. The method of Embodiment 1 wherein the cyclooxygenase-2 nonselective inhibitor is D-1 to D-5, D-6 to D-10, D-11 to D-15, D-16 to D-20, D-21 to D-25, D-26 to D-30, D-31 to D-35, D-36 to D-40, D-41 to D-45, D-46 to D-50, D-51 to D-55, D-56 to D-60, D-61 to D-65, D-66 to D-70, D-71 to D-75, D-76 to D-80, D-81 to D-85, D-86 to D-90, D-91 to D-95, D-96 to D-100, D-101 to D-105, D-106 to D-110, D-111 to D-115, D-116 to D-120, D-121 to D-125, D-126 to D-130, D-131 to D-135, D-136 to D-140, D-141 to D-145, D-146 to D-150, D-151 to D-155, D-156 to D-160, D-161 to D-165, D-166 to D-170, D-171 to D-175, D-176 to D-180, D-181 to D-185, D-186 to D-190, D-191 to D-195, D-196 to D-200, D-201 to D-205, D-206 to D-210, D-211 to D-215, D-216 to D-220, D-221 to D-225, D-226 to D-230, D-231 to D-232, or a pharmaceutically acceptable salt or derivative or prodrug thereof.

9. The method of Embodiment 1 wherein the cyclooxygenase-2 selective inhibitor is selected from the group consisting of meloxicam, celecoxib, valdecoxib, deracoxib, rofecoxib, etoricoxib (MK-663), 4-cyclohexyl-5-[3-fluoro-4-(methylsulphonyl)phenyl]-2-methyl-oxazole (JTE-522), and 6-[[5-(4-chlorobenzoyl)-1,4-dimethyl-1H-pyrrol-2-yl]methyl]-3(2H)-pyridazinone (RS 57067), or a pharmaceutically acceptable salt or derivative or prodrug thereof.

10. The method of Embodiment 9 wherein the cyclooxygenase-2 selective inhibitor is celecoxib.

11. The method of Embodiment 9 wherein the cyclooxygenase-2 selective inhibitor is rofecoxib.

12. The method of embodiment 9 wherein parecoxib, CAS 198470-84-7, is employed as a prodrug and source of the cyclooxygenase-2 selective inhibitor valdecoxib.

13. The method of Embodiment 1 wherein the cyclooxygenase-2 selective inhibitor is a substituted benzopyran or a pharmaceutically acceptable salt or derivative or prodrug thereof.

14. The method of Embodiment 1 wherein the cyclooxygenase-2 selective inhibitor is a substituted benzopyran analog selected from the group consisting of substituted benzothiopyrans, dihydroquinolines, and dihydronaphthalenes, or a pharmaceutically acceptable salt or derivative or prodrug thereof.

15. The method of Embodiments 7-14 wherein the condition is selected from the group consisting of gout, pancreatitis, cholelithiasis, biliary obstruction, ulcerative colitis, Crohn's disease, coronary artery disease, aneurysm, arteriosclerosis, atherosclerosis, myocardial infarction, embolism, stroke, thrombosis, angina, coronary plaque inflammation, bacterial-induced inflammation, viral induced inflammation, and inflammation wherein the inflammation is associated with a surgical procedure involving an artery, a vein or a capillary.

16. The method of Embodiment 1 wherein the apical sodium bile acid transport inhibitor is a substituted benzothiepine compound.

17. The method of Embodiment 1 wherein the apical sodium bile acid transport inhibitor is a substituted benzothiazepine compound.

18. The method of Embodiments 16-17 wherein the condition is selected from the group consisting of gout, pancreatitis, cholelithiasis, biliary obstruction, ulcerative colitis, Crohn's disease, coronary artery disease, aneurysm, arteriosclerosis, atherosclerosis, myocardial infarction, embolism, stroke, thrombosis., angina, coronary plaque inflammation, bacterial-induced inflammation, viral induced inflammation, and inflammation wherein the inflammation is associated with a surgical procedure involving an artery, a vein or a capillary.

19. The method of Embodiment 1 further comprising treating the subject with an amount of an HMG-CoA reductase inhibitor wherein the amount of the apical sodium co-dependent bile acid transport inhibitor and the amount of the cyclooxygenase-2 selective inhibitor and the amount of the HMG-CoA reductase inhibitor together constitute a hypercholesterolemia-related condition effective amount or an inflammation-related condition effective amount of the apical sodium co-dependent bile acid transport inhibitor, the cyclooxygenase-2 selective inhibitor and the HMG-CoA reductase inhibitor.

20. The method of Embodiment 19 wherein the HMG-CoA reductase inhibitor is selected from the group consisting of fluvastatin, lovastatin, pravastatin, simvastatin, atorvastatin, cerivastatin, bervastatin, rosuvastatin, and itavastatin, or a pharmaceutically acceptable salt or ester or lactone thereof.

21. The method of Embodiment 20 wherein the HMG-CoA reductase inhibitor is fluvastatin.

22. The method of Embodiment 20 wherein the HMG-CoA reductase inhibitor is lovastatin.

23. The method of Embodiment 20 wherein the HMG-CoA reductase inhibitor is pravastatin.

24. The method of Embodiment 20 wherein the HMG-CoA reductase inhibitor is simvastatin.

25. The method of Embodiment 20 wherein the HMG-CoA reductase inhibitor is atorvastatin.

26. The method of Embodiment 20 wherein the HMG-CoA reductase inhibitor is cerivastatin.

27. The method of Embodiment 20 wherein the HMG-CoA reductase inhibitor is bervastatin.

28. The method of Embodiment 20 wherein the HMG-CoA reductase inhibitor is rosuvastatin.

29. The method of Embodiment 20 wherein the HKG-CoA reductase inhibitor is itavastatin.

30. The method of Embodiments 19-29 wherein the condition is selected from the group consisting of gout, pancreatitis, cholelithiasis, biliary obstruction, ulcerative colitis, Crohn's disease, coronary artery disease, aneurysm, arteriosclerosis, atherosclerosis, myocardial infarction, embolism, stroke, thrombosis, angina, coronary plaque inflammation, bacterial-induced inflammation, viral induced inflammation, and inflammation wherein the inflammation is associated with a surgical procedure involving an artery, a vein or a capillary.

31. A pharmaceutical combination comprising an amount of an apical sodium co-dependent bile acid transport inhibitor, an amount of a cyclooxygenase-2 selective inhibitor or prodrug, and a pharmaceutically acceptable carrier, wherein the amount of the apical sodium co-dependent bile acid transport inhibitor and the amount of the cyclooxygenase-2 selective inhibitor together constitute a hypercholesterolemia-related condition effective amount or an inflammation-related condition effective amount of the apical sodium co-dependent bile acid transport inhibitor and the cyclooxygenase-2 selective inhibitor.

32. The combination of Embodiment 31 wherein the cyclooxygenase-2 selective inhibitor is D-1, D-2, D-3, D-4, D-5, D-6, D-7, D-8, D-9, D-10, D-11, D-12, D-13, D-14, D-15, D-16, D-17, celecoxib (D-18), D-19, D-20, rofecoxib (D-21), D-22, D-23, D-24, D-25, D-26, D-27, D-28, D-29, D-30, D-31, D-32, D-33, D-34, D-35, D-36, D-37, D-38, D-39, D-40, D-41, D-42, D-43, D-44, D-45, D-46, D-47, D-48, D-49, D-50, D-51, D-52, D-53, D-54, D-55, D-56, D-57, D-58, D-59, D-60, D-61, D-62, D-63, D-64, D-65, D-66, D-67, D-68, D-69, D-70, D-71, D-72, D-73, D-74, D-75, D-76, D-77, D-78, D-79, D-80, D-81, D-82, D-83, D-84, D-85, D-86, D-87, D-88, D-89, D-90, D-91, D-92, D-93, D-94, D-95, D-96, D-97, D-98, D-99, D-100, D-101, D-102, D-103, D-104, D-105, D-106, D-107, D-108, D-109, D-110, D-111, D-112, D-113, D-114, D-115, D-116, D-117, D-118, D-119, D-120, D-121, D-122, D-123, D-124, D-125, D-126, D-127, D-128, D-129, D-130, D-131, D-132, D-133, D-134, D-135, D-136, D-137, D-138, D-139, D-140, D-141, D-142, D-143, D-144, D-145, D-146, D-147, D-148, D-149, D-150, D-151, D-152, D-153, D-154, D-155, D-156, D-157, D-158, D-159, D-160, D-161, D-162, D-163, D-164, D-165, D-166, D-167, D-168, D-169, D-170, D-171, D-172, D-173, D-174, D-175, D-176, D-177, D-178, D-179, D-180, D-181, D-182, D-183, D-184, D-185, D-186, D-187, D-188, D-189, D-190, D-191, D-192, D-193, D-194, D-195, D-196, D-197, D-198, D-199, D-200, D-201, D-202, D-203, D-204, D-205, D-206, D-207, D-208, D-209, D-210, D-211, D-212, D-213, D-214, D-215, D-216, D-217, D-218, D-219, D-220, D-221, D-222, D-223, D-224, D-225, D-226, D-227, D-228, D-229, D-230, D-231, D-232, or a pharmaceutically acceptable salt or derivative or prodrug thereof.

33. The combination of Embodiment 31 wherein the cyclooxygenase-2 selective inhibitor is D-1 to D-5, D-6 to D-10, D-11 to D-15, D-16 to D-20, D-21 to D-25, D-26 to D-30, D-31 to D-35, D-36 to D-40, D-41 to D-45, D-46 to D-50, D-51 to D-55, D-56 to D-60, D-61 to D-65, D-66 to D-70, D-71 to D-75, D-76 to D-80, D-81 to D-85, D-86 to D-90, D-91 to D-95, D-96 to D-100, D-101 to D-105, D-106 to D-110, D-111 to D-115, D-116 to D-120, D-121 to D-125, D-126 to D-130, D-131 to D-135, D-136 to D-140, D-141 to D-145, D-146 to D-150, D-151 to D-155, D-156 to D-160, D-161 to D-165, D-166 to D-170, D-171 to D-175, D-176 to D-180, D-181 to D-185, D-186 to D-190, D-191 to D-195, D-196 to D-200, D-201 to D-205, D-206 to D-210, D-211 to D-215, D-216 to D-220, D-221 to D-225, D-226 to D-230, D-231 to D-232, or a pharmaceutically acceptable salt or derivative or prodrug thereof.

34. The combination of Embodiment 31 wherein the cyclooxygenase-2 selective inhibitor is selected from the group consisting of meloxicam, celecoxib, valdecoxib, deracoxib, rofecoxib, etoricoxib (MK-663), 4-cyclohexyl-5-[3-fluoro-4-(methylsulphonyl)phenyl]-2-methyl-oxazole (JTE-522), and 6-[[5-(4-chlorobenzoyl)-1,4-dimethyl-1H-pyrrol-2-yl]methyl]-3(2H)-pyridazinone (RS 57067), or a pharmaceutically acceptable salt or derivative or prodrug thereof.

35. The combination of Embodiment 34 wherein the cyclooxygenase-2 selective inhibitor is celecoxib.

36. The combination of Embodiment 34 wherein the cyclooxygenase-2 selective inhibitor is rofecoxib.

37. The combination of embodiment 34 wherein parecoxib, CAS 198470-84-7, is employed as a prodrug and source of the cyclooxygenase-2 selective inhibitor valdecoxib.

38. The combination of Embodiment 31 wherein the cyclooxygenase-2 selective inhibitor is a substituted benzopyran or a pharmaceutically acceptable salt or derivative or prodrug thereof.

39. The combination of Embodiment 34 wherein the cyclooxygenase-2 selective inhibitor is a substituted benzopyran analog selected from the group consisting of substituted benzothiopyrans, dihydroquinolines, and dihydronaphthalenes, or a pharmaceutically acceptable salt or derivative or prodrug thereof.

40. The combination of Embodiment 31 wherein the apical sodium bile acid transport inhibitor is a substituted benzothiepine compound.

41. The combination of Embodiment 31 wherein the apical sodium bile acid transport inhibitor is a substituted benzothiazepine compound.

42. A process for preparing the pharmaceutical combination of Embodiment 31 comprising combining an amount of the apical sodium co-dependent bile acid transport inhibitor, an amount of a cyclooxygenase-2 selective inhibitor or prodrug, and a pharmaceutically acceptable carrier.

43. The combination of Embodiment 31 further comprising an amount of an HMG-CoA reductase inhibitor wherein the amount of the apical sodium co-dependent bile acid transport inhibitor, the amount of the cyclooxygenase-2 selective inhibitor and the amount of the HMG-CoA reductase inhibitor together constitute a hypercholesterolemia-related condition effective amount or an inflammation-related condition effective amount of the apical sodium co-dependent bile acid transport inhibitor and the cyclooxygenase-2 selective inhibitor and the HMG-CoA reductase inhibitor.

44. The combination of Embodiment 43 wherein the HMG-CoA reductase inhibitor is selected from the group consisting of fluvastatin, lovastatin, pravastatin, simvastatin, atorvastatin, cerivastatin, bervastatin, rosuvastatin, and itavastatin, or a pharmaceutically acceptable salt or ester or lactone thereof.

45. The combination of Embodiment 44 wherein the HMG-CoA reductase inhibitor is fluvastatin.

46. The combination of Embodiment 44 wherein the HMG-CoA reductase inhibitor is lovastatin.

47. The combination of Embodiment 44 wherein the HMG-CoA reductase inhibitor is pravastatin.

48. The combination of Embodiment 44 wherein the HMG-CoA reductase inhibitor is simvastatin.

49. The combination of Embodiment 44 wherein the HMG-CoA reductase inhibitor is atorvastatin.

50. The combination of Embodiment 44 wherein the HMG-CoA reductase inhibitor is cerivastatin.

51. The combination of Embodiment 44 wherein the HMG-CoA reductase inhibitor is bervastatin.

52. The combination of Embodiment 44 wherein the HMG-CoA reductase inhibitor is rosuvastatin.

53. The combination method of Embodiment 44 wherein the HMG-CoA reductase inhibitor is itavastatin.

54. The process of Embodiment 42 further comprising combining an amount of an HMG-CoA reductase inhibitor, an amount of the apical sodium co-dependent bile acid transport inhibitor, an amount of a cyclooxygenase-2 selective inhibitor or prodrug, and a pharmaceutically acceptable carrier.

55. A kit comprised of an amount of an apical sodium co-dependent bile acid transport inhibitor in a dosage formulation and an amount of a cyclooxygenase-2 selective inhibitor or prodrug in a separate dosage formulation wherein the amount of the apical sodium co-dependent bile acid transport inhibitor and the amount of the cyclooxygenase-2 selective inhibitor together constitute a hypercholesterolemia-related condition effective amount or an inflammation-related condition effective amount of the apical sodium co-dependent bile acid transport inhibitor and the cyclooxygenase-2 selective inhibitor.

56. The kit of Embodiment 55 wherein the cyclooxygenase-2 selective inhibitor is D-1, D-2, D-3, D-4, D-5, D-6, D-7, D-8, D-9, D-10, D-11, D-12, D-13, D-14, D-15, D-16, D-17, celecoxib (D-18), D-19, D-20, rofecoxib (D-21), D-22, D-23, D-24, D-25, D-26, D-27, D-28, D-29, D-30, D-31, D-32, D-33, D-34, D-35, D-36, D-37, D-38, D-39, D-40, D-41, D-42, D-43, D-44, D-45, D-46, D-47, D-48, D-49, D-50, D-51, D-52, D-53, D-54, D-55, D-56, D-57, D-58, D-59, D-60, D-61, D-62, D-63, D-64, D-65, D-66, D-67, D-68, D-69, D-70, D-71, D-72, D-73, D-74, D-75, D-76, D-77, D-78, D-79, D-80, D-81, D-82, D-83, D-84, D-85, D-86, D-87, D-88, D-89, D-90, D-91, D-92, D-93, D-94, D-95, D-96, D-97, D-98, D-99, D-100, D-101, D-102, D-103, D-104, D-105, D-106, D-107, D-108, D-109, D-110, D-111, D-112, D-113, D-114, D-115, D-116, D-117, D-118, D-119, D-120, D-121, D-122, D-123, D-124, D-125, D-126, D-127, D-128, D-129, D-130, D-131, D-132, D-133, D-134, D-135, D-136, D-137, D-138, D-139, D-140, D-141, D-142, D-143, D-144, D-145, D-146, D-147, D-148, D-149, D-150, D-151, D-152, D-153, D-154, D-155, D-156, D-157, D-158, D-159, D-160, D-161, D-162, D-163, D-164, D-165, D-166, D-167, D-168, D-169, D-170, D-171, D-172, D-173, D-174, D-175, D-176, D-177, D-178, D-179, D-180, D-181, D-182, D-183, D-184, D-185, D-186, D-187, D-188, D-189, D-190, D-191, D-192, D-193, D-194, D-195, D-196, D-197, D-198, D-199, D-200, D-201, D-202, D-203, D-204, D-205, D-206, D-207, D-208, D-209, D-210, D-211, D-212, D-213, D-214, D-215, D-216, D-217, D-218, D-219, D-220, D-221, D-222, D-223, D-224, D-225, D-226, D-227, D-228, D-229, D-230, D-231, D-232, or a pharmaceutically acceptable salt or derivative or prodrug thereof.

57. The kit of Embodiment 55 wherein the cyclooxygenase-2 selective inhibitor is D-1 to D-5, D-6 to D-10, D-11 to D-15, D-16 to D-20, D-21 to D-25, D-26 to D-30, D-31 to D-35, D-36 to D-40, D-41 to D-45, D-46 to D-50, D-51 to D-55, D-56 to D-60, D-61 to D-65, D-66 to D-70, D-71 to D-75, D-76 to D-80, D-81 to D-85, D-86 to D-90, D-91 to D-95, D-96 to D-100, D-101 to D-105, D-106 to D-110, D-111 to D-115, D-116 to D-120, D-121 to D-125, D-126 to D-130, D-131 to D-135, D-136 to D-140, D-141 to D-145, D-146 to D-150, D-151 to D-155, D-156 to D-160, D-161 to D-165, D-166 to D-170, D-171 to D-175, D-176 to D-180, D-181 to D-185, D-186 to D-190, D-191 to D-195, D-196 to D-200, D-201 to D-205, D-206 to D-210, D-211 to D-215, D-216 to D-220, D-221 to D-225, D-226 to D-230, D-231 to D-232, or a pharmaceutically acceptable salt or derivative or prodrug thereof.

58. The kit of Embodiment 55 wherein the cyclooxygenase-2 selective inhibitor is selected from the group consisting of meloxicam, celecoxib, valdecoxib, deracoxib, rofecoxib, etoricoxib (MK-663), 4-cyclohexyl-5-[3-fluoro-4-(methylsulphonyl)phenyl]-2-methyl-oxazole (JTE-522), and 6-[[5-(4-chlorobenzoyl)-1,4-dimethyl-1H-pyrrol-2-yl]methyl]-3(2H)-pyridazinone (RS 57067), or a pharmaceutically acceptable salt or derivative or prodrug thereof.

59. The kit of Embodiment 58 wherein the cyclooxygenase-2 selective inhibitor is celecoxib.

60. The kit of Embodiment 58 wherein the cyclooxygenase-2 selective inhibitor is rofedoxib.

61. The kit of embodiment 58 wherein parecoxib, CAS 198470-84-7, is employed as a prodrug and source of the cyclooxygenase-2 selective inhibitor valdecoxib.

62. The kit of Embodiment 55 wherein the cyclooxygenase-2 selective inhibitor is a substituted benzopyran or a pharmaceutically acceptable salt or derivative or prodrug thereof.

63. The kit of Embodiment 55 wherein the cyclooxygenase-2 selective inhibitor is a substituted benzopyran analog selected from the group consisting of substituted benzothiopyrans, dihydroquinolines, and dihydronaphthalenes, or a pharmaceutically acceptable salt or derivative or prodrug thereof.

64. The kit of Embodiment 55 wherein the apical sodium bile acid transport inhibitor is a substituted benzothiepine compound.

65. The kit of Embodiment 55 wherein the apical sodium bile acid transport inhibitor is a substituted benzothiazepine compound.

66. The kit of Embodiment 55 further comprising an amount of an HMG-CoA reductase inhibitor wherein the amount of the apical sodium co-dependent bile acid transport inhibitor, the amount of the cyclooxygenase-2 selective inhibitor and the amount of the HMG-CoA reductase inhibitor together constitute a hypercholesterolemia-related condition effective amount or an inflammation-related condition effective amount of the apical sodium co-dependent bile acid transport inhibitor, the cyclooxygenase-2 selective inhibitor and the HMG-CoA reductase inhibitor.

67. The kit of Embodiment 66 wherein the HMG-CoA reductase inhibitor is selected from the group consisting of fluvastatin, lovastatin, pravastatin, simvastatin, atorvastatin, cerivastatin, bervastatin, rosuvastatin, and itavastatin, or a pharmaceutically acceptable salt or ester or lactone thereof.

68. The kit of Embodiment 67 wherein the HMG-CoA reductase inhibitor is fluvastatin.

69. The kit of Embodiment 67 wherein the HMG-CoA reductase inhibitor is lovastatin.

70. The kit of Embodiment 67 wherein the HMG-CoA reductase inhibitor is pravastatin.

71. The kit of Embodiment 67 wherein the HMG-CoA reductase inhibitor is simvastatin.

72. The kit of Embodiment 67 wherein the HMG-CoA reductase inhibitor is atorvastatin.

73. The kit of Embodiment 67 wherein the HMG-CoA reductase inhibitor is cerivastatin.

74. The kit of Embodiment 67 wherein the HMG-CoA reductase inhibitor is bervastatin.

75. The kit of Embodiment 67 wherein the HMG-CoA reductase inhibitor is rosuvastatin.

76. The kit of Embodiment 67 wherein the HMG-CoA reductase inhibitor is itavastatin.

77. A method for treating or preventing a hypercholesterolemia-related or an inflammation-related condition in a subject in need of such treatment or prevention, comprising treating the subject with an amount of an apical sodium co-dependent bile acid transport inhibitor and an amount of a chromene cyclooxygenase-2 selective inhibitor or prodrug, wherein the amount of the apical sodium co-dependent bile acid transport inhibitor and the amount of the chromene cyclooxygenase-2 selective inhibitor together constitute a hypercholesterolemia-related condition effective amount or an inflammation-related condition effective amount of the apical sodium co-dependent bile acid transport inhibitor and the chromene cyclooxygenase-2 selective inhibitor.

78. A method for treating or preventing a hypercholesterolemia-related or an inflammation-related condition in a subject in need of such treatment or prevention, comprising treating the subject with an amount of an HMG Co-A reductase inhibitor and an amount of a chromene cyclooxygenase-2 selective inhibitor or prodrug, wherein the amount of the HMG Co-A reductase inhibitor and the amount of the chromene cyclooxygenase-2 selective inhibitor together constitute a hypercholesterolemia-related condition effective amount or an inflammation-related condition effective amount of the HMG Co-A reductase inhibitor and the chromene cyclooxygenase-2 selective inhibitor.

The examples herein can be performed by substituting the generically or specifically described therapeutic compounds or inert ingredients for those used in the preceding examples.

The invention being thus described, it is apparent that the same can be varied in many ways. Such variations are not to be regarded as a departure from the spirit and scope of the present invention, and all such modifications and equivalents as would be obvious to one skilled in the art are intended to be included within the scope of the following claims.

What is claimed is:

1. A method for treating or preventing a hypercholesterolemia-related or an inflammation-related condition in a subject in need of such treatment or prevention, comprising treating the subject with an amount of an apical sodium co-dependent bile acid transport inhibitor, an amount of a cyclooxygenase-2 selective inhibitor or prodrug, wherein the amount of the apical sodium co-dependent bile acid transport inhibitor, the amount of the cyclooxygenase-2 selective inhibitor together constitute a hypercholesterolemia-related condition effective amount or an inflammation-related condition effective amount of the apical sodium co-dependent bile acid transport inhibitor and the cyclooxygenase-2 inhibitor.

2. The method of claim 1 wherein the amount of the apical sodium co-dependent bile acid transport inhibitor and the amount of the cyclooxygenase-2 selective inhibitor together constitute a hypercholesterolemia-related condition effective amount of the apical sodium co-dependent bile acid transport inhibitor and the cyclooxygenase inhibitor.

3. The method of claim 1 wherein the amount of the apical sodium co-dependent bile acid transport inhibitor and the amount of the cyclooxygenase-2 selective inhibitor together constitute an inflammation-related condition effective amount of the apical sodium co-dependent bile acid transport inhibitor and the cyclooxygenase-2 selective inhibitor.

4. The method of claim 1 wherein the condition is selected from the group consisting of gout, pancreatitis, cholelithiasis, biliary obstruction, ulcerative colitis, Crohn's disease, coronary artery disease, aneurysm, arteriosclerosis, atherosclerosis, myocardial infarction, embolism, stroke, thrombosis, angina, coronary plaque inflammation, bacterial-induced inflammation, viral induced inflammation, and inflammation wherein the inflammation is associated with a surgical procedure involving an artery, a vein or a capillary.

5. The method of claim 4 wherein the condition is selected from the group consisting of coronary artery disease, atherosclerosis, and thrombosis.

6. The method of claim 5 wherein the condition is coronary artery disease.

7. The method of claim 1 wherein the cyclooxygenase-2 selective inhibitor is
[2-(2,4-Dichloro-6-ethyl-3,5-dimethyl-phenylamino)-5-propyl-phenyl]-acetic acid (D-1);
6-[[5-(4-chlorobenzoyl)-1,4-dimethyl-1H-pyrrol-2-yl]methyl]-3(2H)-pyridazinone or RS 57067 (D-2);
6-Nitro-2-trifluoromethyl-2H-1-benzopyran-3-carboxylic acid (D-3);
6-Chloro-8-methyl-2-trifluoromethyl-2H-1-benzopyran-3-carboxylic acid (D-4);
((S)-6-chloro-7-(1,1-dimethylethyl)-2-trifluoromethyl-2H-1-benzopyran-3-carboxylic acid (D-5);
2-Trifluoromethyl-2H-naphtho[2,3-b]pyran-3-carboxylic acid (D-6);
6-Chloro-7-(4-nitrophenoxy)-2-(trifluoromethyl)-2H-1-benzopyran-3-carboxylic acid (D-7);
((S)-6,8-Dichloro-2-(trifluoromethyl)-2H-1-benzopyran-3-carboxylic acid (D-8);
6-Chloro-2-(trifluoromethyl)-4-phenyl-2H-1-benzopyran-3-carboxylic acid (D-9);
6-(4-Hydroxybenzoyl)-2-(trifluoromethyl)-2H-1-benzopyran-3-carboxylic acid (D-10);
2-(Trifluoromethyl)-6-[(trifluoromethyl)thio]-2H-1-benzothiopyran-3-carboxylic acid (D-11);
6,8-Dichloro-2-trifluoromethyl-2H-1-benzothiopyran-3-carboxylic acid (D-12);
6-(1,1-Dimethylethyl)-2-(trifluoromethyl)-2H-1-benzothiopyran-3-carboxylic acid (D-13);
6,7-Difluoro-1,2-dihydro-2-(trifluoromethyl)-3-quinolinecarboxylic acid (D-14);
6-Chloro-1,2-dihydro-1-methyl-2-(trifluoromethyl)-3-quinolinecarboxylic acid (D-15);
6-Chloro-2-(trifluoromethyl)-1,2-dihydro[1,8]naphthyridine-3-carboxylic acid (D-16);
((S)-6-Chloro-1,2-dihydro-2-(trifluoromethyl)-3-quinolinecarboxylic acid (D-17);
celecoxib (D-18);
valdecoxib (D-19);
deracoxib (D-20);
rofecoxib (D-21);
etoricoxib (D-22);
JTE-522 (D-23);
parecoxib (D-24)
ABT-963 (D-25);
N-(2-cyclohexyloxy-4-nitro-phenyl)-methanesulfonamide or NS-398 (D-26);
6-chloro-2-trifluoromethyl-2H-1-benzopyran-3-carboxylic acid (D-27);
6-chloro-7-methyl-2-trifluoromethyl-2H-1-benzopyran-3-carboxylic acid (D-28);
8-(1-methylethyl)-2-trifluoromethyl-2H-1-benzopyran-3-carboxylic acid (D-29);
6-chloro-8-(1-methylethyl)-2-trifluoromethyl-2H-1-benzopyran-3-carboxylic acid (D-30);
2-trifluoromethyl-3H-naphthopyran-3-carboxylic acid (D-31);
7-(1,1-dimethylethyl)-2-trifluoromethyl-2H-1-benzopyran-3-carboxylic acid (D-32);
6-bromo-2-trifluoromethyl-2H-1-benzopyran-3-carboxylic acid (D-33);
8-chloro-2-trifluoromethyl-2H-1-benzopyran-3-carboxylic acid (D-34);
6-trifluoromethoxy-2-trifluoromethyl-2H-1-benzopyran-3-carboxylic acid (D-35);
5,7-dichloro-2-trifluoromethyl-2H-1-benzopyran-3-carboxylic acid (D-36);
8-phenyl-2-trifluoromethyl-2H-1-benzopyran-3-carboxylic acid (D-37);
7,8-dimethyl-2-trifluoromethyl-2H-1-benzopyran-3-carboxylic acid (D-38);
6,8-bis(dimethylethyl)-2-trifluoromethyl-2H-1-benzopyran-3-carboxylic acid (D-39);
7-(1-methylethyl)-2-trifluoromethyl-2H-1-benzopyran-3-carboxylic acid (D-40);
7-phenyl-2-trifluoromethyl-2H-1-benzopyran-3-carboxylic acid (D-41);
6-chloro-7-ethyl-2-trifluoromethyl-2H-1-benzopyran-3-carboxylic acid (D-42);
6-chloro-8-ethyl-2-trifluoromethyl-2H-1-benzopyran-3-carboxylic acid (D-43);
6-chloro-7-phenyl-2-trifluoromethyl-2H-1-benzopyran-3-carboxylic acid (D-44);
6,7-dichloro-2-trifluoromethyl-2H-1-benzopyran-3-carboxylic acid (D-45);
6,8-dichloro-2-trifluoromethyl-2H-1-benzopyran-3-carboxylic acid (D-46);
2-trifluoromethyl-3H-naptho[2,1-b]pyran-3-carboxylic acid (D-29);
8-chloro-6-methyl-2-trifluoromethyl-2H-1-benzopyran-3-carboxylic acid (D-48
8-chloro-6-methoxy-2-trifluoromethyl-2H-1-benzopyran-3-carboxylic acid (D-49);
6-bromo-8-chloro-2-trifluoromethyl-2H-1-benzopyran-3-carboxylic acid (D-50);
8-bromo-6-fluoro-2-trifluoromethyl-2H-1-benzopyran-3-carboxylic acid (D-51);
8-bromo-6-methyl-2-trifluoromethyl-2H-1-benzopyran-3-carboxylic acid (D-52);
8-bromo-5-fluoro-2-trifluoromethyl-2H-1-benzopyran-3-carboxylic acid (D-53);
6-chloro-8-fluoro-2-trifluoromethyl-2H-1-benzopyran-3-carboxylic acid (D-54);
6-bromo-8-methoxy-2-trifluoromethyl-2H-1-benzopyran-3-carboxylic acid (D-55);
6-[[(phenylmethyl)amino]sulfonyl]-2-trifluoromethyl-2H-1-benzopyran-3-carboxylic acid (D-56);
6-[(dimethylamino)sulfonyl]-2-trifluoromethyl-2H-1-benzopyran-3-carboxylic acid (D-57);
6-[(methylamino)sulfonyl]-2-trifluoromethyl-2H-1-benzopyran-3-carboxylic acid (D-58);
6-[(4-morpholino)sulfonyl]-2-trifluoromethyl-2H-1-benzopyran-3-carboxylic acid (D-59);
6-[(1,1-dimethylethyl)aminosulfonyl]-2-trifluoromethyl-2H-1-benzopyran-3-carboxylic acid (D-60);
6-[(2-methylpropyl)aminosulfonyl]-2-trifluoromethyl-2H-1-benzopyran-3-carboxylic acid (D-61);
6-methylsulfonyl-2-trifluoromethyl-2H-1-benzopyran-3-carboxylic acid (D-62);
8-chloro-6-[[(phenylmethyl)amino]sulfonyl]-2-trifluoromethyl-2H-1-benzopyran-3-carboxylic acid (D-63);
6-phenylacetyl-2-trifluoromethyl-2H-1-benzopyran-3-carboxylic acid (D-64);
6,8-dibromo-2-trifluoromethyl-2H-1-benzopyran-3-carboxylic acid (D-65);
8-chloro-5,6-dimethyl-2-trifluoromethyl-2H-1-benzopyran-3-carboxylic acid (D-66);
6,8-dichloro-(S)-2-trifluoromethyl-2H-1-benzopyran-3-carboxylic acid (D-67);
6-benzylsulfonyl-2-trifluoromethyl-2H-1-benzopyran-3-carboxylic acid (D-68);
6-[[N-(2-furylmethyl)amino]sulfonyl]-2-trifluoromethyl-2H-1-benzopyran-3-carboxylic acid (D-69);
6-[[N-(2-phenylethyl)amino]sulfonyl]-2-trifluoromethyl-2H-1-benzopyran-3-carboxylic acid (D-70);
6-iodo-2-trifluoromethyl-2H-1-benzopyran-3-carboxylic acid (D-71);
7-(1,1-dimethylethyl)-2-pentafluoroethyl-2H-1-benzopyran-3-carboxylic acid (D-72);
6-chloro-2-trifluoromethyl-2H-1-benzothiopyran-3-carboxylic acid (D-73);
BMS-347070 (D-74);
8-acetyl-3-(4-fluorophenyl)-2-(4-methylsulfonyl)phenyl-imidazo(1,2-a)pyridine (D-75);
5,5-dimethyl-4-(4-methylsulfonyl)phenyl-3-phenyl-2-(5H)-furanone (D-76);
5-(4-fluorophenyl)-1-[4-(methylsulfonyl)phenyl]-3-(trifluoromethyl)pyrazole (D-77);
4-(4-fluorophenyl)-5-[4-(methylsulfonyl)phenyl]-1-phenyl-3-(trifluoromethyl)pyrazole (D-78);
4-(5-(4-chlorophenyl)-3-(4-methoxyphenyl)-1H-pyrazol-1-yl)benzenesulfonamide(D-79);
4-(3,5-bis(4-methylphenyl)-1H-pyrazol-1-yl)benzenesulfonamide(D-80);
4-(5-(4-chlorophenyl)-3-phenyl-1H-pyrazol-1-yl)benzenesulfonamide(D-81);
4-(3,5-bis(4-methoxyphenyl)-1H-pyrazol-1-yl)benzenesulfonamide(D-82);
4-(5-(4-chlorophenyl)-3-(4-methylphenyl)-1H-pyrazol-1-yl)benzenesulfonamide(D-83);
4-(5-(4-chlorophenyl)-3-(4-nitrophenyl)-1H-pyrazol-1-yl)benzenesulfonamide(D-84);
4-(5-(4-chlorophenyl)-3-(5-chloro-2-thienyl)-1H-pyrazol-1-yl)benzenesulfonamide(D-85);
4-(4-chloro-3,5-diphenyl-1H-pyrazol-1-yl)benzenesulfonamide(D-86);
4-[5-(4-chlorophenyl)-3-(trifluoromethyl)-1H-pyrazol-1-yl]benzenesulfonamide(D-87);
4-[5-phenyl-3-(trifluoromethyl)-1H-pyrazol-1-yl]benzenesulfonamide (D-88);
4-[5-(4-fluorophenyl)-3-(trifluoromethyl)-1H-pyrazol-1-yl]benzenesulfonamide (D-89);
4-[5-(4-methoxyphenyl)-3-(trifluoromethyl)-1H-pyrazol-1-yl]benzenesulfonamide (D-90);
4-[5-(4-chlorophenyl)-3-(difluoromethyl)-1H-pyrazol-1-yl]benzenesulfonamide (D-91);
4-[5-(4-methylphenyl)-3-(trifluoromethyl)-1H-pyrazol-1-yl]benzenesulfonamide (D-92);
4-[4-chloro-5-(4-chlorophenyl)-3-(trifluoromethyl)-1H-pyrazol-1-yl]benzenesulfonamide (D-93);
4-[3-(difluoromethyl)-5-(4-methylphenyl)-1H-pyrazol-1-yl]benzenesulfonamide (D-94);
4-[3-(difluoromethyl)-5-phenyl-1H-pyrazol-1-yl]benzenesulfonamide (D-95);
4-[3-(difluoromethyl)-5-(4-methoxyphenyl)-1H-pyrazol-1-yl]benzenesulfonamide (D-96);
4-[3-cyano-5-(4-fluorophenyl)-1H-pyrazol-1-yl]benzenesulfonamide (D-97);
4-[3-(difluoromethyl)-5-(3-fluoro-4-methoxyphenyl)-1H-pyrazol-1-yl]benzenesulfonamide (D-98);
4-[5-(3-fluoro-4-methoxyphenyl)-3-(trifluoromethyl)-1H-pyrazol-1-yl]benzenesulfonamide (D-99);
4-[4-chloro-5-phenyl-1H-pyrazol-1-yl]benzenesulfonamide (D-100);
4-[5-(4-chlorophenyl)-3-(hydroxymethyl)-1H-pyrazol-1-yl]benzenesulfonamide (D-101);
4-[5-(4-(N,N-dimethylamino)phenyl)-3-(trifluoromethyl)-1H-pyrazol-1-yl]benzenesulfonamide (D-102);
5-(4-fluorophenyl)-6-[4-(methylsulfonyl)phenyl]spiro[2.4]hept-5-ene (D-103);
4-[6-(4-fluorophenyl)spiro[2.4]hept-5-en-5-yl]benzenesulfonamide (D-104);
6-(4-fluorophenyl)-7-[4-(methylsulfonyl)phenyl]spiro[3.4]oct-6-ene (D-105);
5-(3-chloro-4-methoxyphenyl)-6-[4-(methylsulfonyl)phenyl]spiro[2.4]hept-5-ene (D-106);
4-[6-(3-chloro-4-methoxyphenyl)spiro[2.4]hept-5-en-5-yl]benzenesulfonamide (D-107);
5-(3,5-dichloro-4-methoxyphenyl)-6-[4--(methylsulfonyl)phenyl]spiro[2.4]hept-5-ene (D-108);
5-(3-chloro-4-fluorophenyl)-6-[4-(methylsulfonyl)phenyl]spiro[2.4]hept-5-ene (D-109);
4-[6-(3,4-dichlorophenyl)spiro[2.4]hept-5-en-5-yl]benzenesulfonamide (D-110);
2-(3-chloro-4-fluorophenyl)-4-(4-fluorophenyl)-5-(4-methylsulfonylphenyl)thiazole (D-111);
2-(2-chlorophenyl)-4-(4-fluorophenyl)-5-(4-methylsulfonylphenyl)thiazole (D-112);
5-(4-fluorophenyl)-4-(4-methylsulfonylphenyl)-2-methylthiazole (D-113);
4-(4-fluorophenyl)-5-(4-methylsulfonylphenyl)-2-trifluoromethylthiazole (D-114);
4-(4-fluorophenyl)-5-(4-methylsulfonylphenyl)-2-(2-thienyl)thiazole (D-115);
4-(4-fluorophenyl)-5-(4-methylsulfonylphenyl)-2-benzylaminothiazole (D-116);
4-(4-fluorophenyl)-5-(4-methylsulfonylphenyl)-2-(1-propylamino)thiazole (D-117);
2-((3,5-dichlorophenoxy)methyl)-4-(4-fluorophenyl)-5-[4-(methylsulfonyl)phenyl]thiazole (D-118);
5-(4-fluorophenyl)-4-(4-methylsulfonylphenyl)-2-trifluoromethylthiazole (D-119);
1-methylsulfonyl-4-[1,1-dimethyl-4-(4-fluorophenyl)cyclopenta-2,4-dien-3-yl]benzene (D-120);
4-[4-(4-fluorophenyl)-1,1-dimethylcyclopenta-2,4-dien-3-yl]benzenesulfonamide (D-121);
5-(4-fluorophenyl)-6-[4-(methylsulfonyl)phenyl]spiro[2.4]hepta-4,6-diene (D-122);
4-[6-(4-fluorophenyl)spiro[2.4]hepta-4,6-dien-5-yl]benzenesulfonamide (D-123);
6-(4-fluorophenyl)-2-methoxy-5-[4-(methylsulfonyl)phenyl]-pyridine-3-carbonitrile (D-124);
2-bromo-6-(4-fluorophenyl)-5-[4-(methylsulfonyl)phenyl]-pyridine-3-carbonitrile (D-125);
6-(4-fluorophenyl)-5-[4-(methylsulfonyl)phenyl]-2-phenyl-pyridine-3-carbonitrile (D-126);
4-[2-(4-methylpyridin-2-yl)-4-(trifluoromethyl)-1H-imidazol-1-yl]benzenesulfonamide (D-127);
4-[2-(5-methylpyridin-3-yl)-4-(trifluoromethyl)-1H-imidazol-1-yl]benzenesulfonamide (D-128);
4-[2-(2-methylpyridin-3-yl)-4-(trifluoromethyl)-1H-imidazol-1-yl]benzenesulfonamide (D-129);
3-[1-[4-(methylsulfonyl)phenyl]-4-(trifluoromethyl)-1H-imidazol-2-yl]pyridine (D-130);
2-[1-[4-(methylsulfonyl)phenyl-4-(trifluoromethyl)-1H-imidazol-2-yl]pyridine (D-131);
2-methyl-4-[1-[4-(methylsulfonyl)phenyl-4-(trifluoromethyl)-1H-imidazol-2-yl]pyridine (D-132);
2-methyl-6-[1-[4-(methylsulfonyl)phenyl-4-(trifluoromethyl)-1H-imidazol-2-yl]pyridine (D-133);
4-[2-(6-methylpyridin-3-yl)-4-(trifluoromethyl)-1H-imidazol-1-yl]benzenesulfonamide (D-134);
2-(3,4-difluorophenyl)-1-[4-(methylsulfonyl)phenyl]-4-(trifluoromethyl)-1H-imidazole (D-135);
4-[2-(4-methylphenyl)-4-(trifluoromethyl)-1H-imidazol-1-yl]benzenesulfonamide (D-136);
2-(4-chlorophenyl)-1-[4-(methylsulfonyl)phenyl]-4-methyl-1H-imidazole (D-137);
2-(4-chlorophenyl)-1-[4-(methylsulfonyl)phenyl]-4-phenyl-1H-imidazole (D-138);
2-(4-chlorophenyl)-4-(4-fluorophenyl)-1-[4-(methylsulfonyl)phenyl]-1H-imidazole (D-139);
2-(3-fluoro-4-methoxyphenyl)-1-[4-(methylsulfonyl)phenyl-4-(trifluoromethyl)-1H-imidazole (D-140);
1-[4-(methylsulfonyl)phenyl]-2-phenyl-4-trifluoromethyl-1H-imidazole (D-141);
2-(4-methylphenyl)-1-[4-(methylsulfonyl)phenyl]-4-trifluoromethyl-1H-imidazole (D-142);
4-[2-(3-chloro-4-methylphenyl)-4-(trifluoromethyl)-1H-imidazol-1-yl]benzenesulfonamide (D-143);
2-(3-fluoro-5-methylphenyl)-1-[4-(methylsulfonyl)phenyl]-4-(trifluoromethyl)-1H-imidazole (D-144);
4-[2-(3-fluoro-5-methylphenyl)-4-(trifluoromethyl)-1H-imidazol-1-yl]benzenesulfonamide (D-145);
2-(3-methylphenyl)-1-[4-(methylsulfonyl)phenyl]-4-trifluoromethyl-1H-imidazole (D-146);
4-[2-(3-methylphenyl)-4-trifluoromethyl-1H-imidazol-1-yl]benzenesulfonamide (D-147);
1-[4-(methylsulfonyl)phenyl]-2-(3-chlorophenyl)-4-trifluoromethyl-1H-imidazole (D-148);
4-[2-(3-chlorophenyl)-4-trifluoromethyl-1H-imidazol-1-yl]benzenesulfonamide (D-149);
4-[2-phenyl-4-trifluoromethyl-1H-imidazol-1-yl]benzenesulfonamide (D-150);
4-[2-(4-methoxy-3-chlorophenyl)-4-trifluoromethyl-1H-imidazol-1-yl]benzenesulfonamide (D-151);
1-allyl-4-(4-fluorophenyl)-3-[4-(methylsulfonyl)phenyl]-5-(trifluoromethyl)-1H-pyrazole (D-152);
4-[1-ethyl-4-(4-fluorophenyl)-5-(trifluoromethyl)-1H-pyrazol-3-yl]benzenesulfonamide (D-153);
N-phenyl-[4-(4-fluorophenyl)-3-[4-(methylsulfonyl)phenyl]-5-(trifluoromethyl)-1H-pyrazol-1-yl]acetamide (D-154);
ethyl [4-(4-fluorophenyl)-3-[4-(methylsulfonyl)phenyl]-5-(trifluoromethyl)-1H-pyrazol-1-yl]acetate (D-155);
4-(4-fluorophenyl)-3-(4-(methylsulfonyl)phenyl]-1-(2-phenylethyl)-1H-pyrazole (D-156);
4-(4-fluorophenyl)-3-[4-(methylsulfonyl)phenyl]-1-(2-phenylethyl)-5-(trifluoromethyl)pyrazole (D-157);
1-ethyl-4-(4-fluorophenyl)-3-[4-(methylsulfonyl)phenyl]-5-(trifluoromethyl)-1H-pyrazole (D-158);
5-(4-fluorophenyl)-4-(4-methylsulfonylphenyl)-2-trifluoromethyl-1H-imidazole (D-159);
4-[4-(methylsulfonyl)phenyl]-5-(2-thiophenyl)-2-(trifluoromethyl)-1H-imidazole (D-160);
5-(4-fluorophenyl)-2-methoxy-4-[4-(methylsulfonyl)phenyl]-6-(trifluoromethyl)pyridine (D-161);
2-ethoxy-5-(4-fluorophenyl)-4-[4-(methylsulfonyl)phenyl]-6-(trifluoromethyl)pyridine (D-162);
5-(4-fluorophenyl)-4-[4-(methylsulfonyl)phenyl]-2-(2-propynyloxy)-6-(trifluoromethyl)pyridine (D-163);
2-bromo-5-(4-fluorophenyl)-4-[4-(methylsulfonyl)phenyl]-6-(trifluoromethyl)pyridine (D-164);
4-[2-(3-chloro-4-methoxyphenyl)-4,5-difluorophenyl]benzenesulfonamide (D-165);
1-(4-fluorophenyl)-2-[4-(methylsulfonyl)phenyl]benzene (D-166);
5-difluoromethyl-4-(4-methylsulfonylphenyl)-3-phenylisoxazole (D-167);
4-[3-ethyl-5-phenylisoxazol-4-yl]benzenesulfonamide (D-168);
4-[5-difluoromethyl-3-phenylisoxazol-4-yl]benzenesulfonamide (D-169);
4-[5-hydroxymethyl-3-phenylisoxazol-4-yl]benzenesulfonamide (D-170);
4-(5-methyl-3-phenyl-isoxazol-4-yl]benzenesulfonamide (D-171);
1-[2-(4-fluorophenyl)cyclopenten-1-yl]-4-(methylsulfonyl)benzene (D-172);
1-[2-(4-fluoro-2-methylphenyl)cyclopenten-1-yl]-4-(methylsulfonyl)benzene (D-173);
1-[2-(4-chlorophenyl)cyclopenten-1-yl]-4-(methylsulfonyl)benzene (D-174);
1-[2-(2,4-dichlorophenyl)cyclopenten-1-yl]-4-(methylsulfonyl)benzene (D-175);
1-[2-(4-trifluoromethylphenyl)cyclopenten-1-yl]-4-(methylsulfonyl)benzene (D-176);
1-[2-(4-methylthiophenyl)cyclopenten-1-yl]-4-(methylsulfonyl)benzene (D-177);
1-[2-(4-fluorophenyl)-4,4-dimethylcyclopenten-1-yl]-4-(methylsulfonyl)benzene (D-178);
4-[2-(4-fluorophenyl)-4,4-dimethylcyclopenten-1-yl]benzenesulfonamide (D-179);
1-[2-(4-chlorophenyl)-4,4-dimethylcyclopenten-1-yl]-4-(methylsulfonyl)benzene (D-180);
4-[2-(4-chlorophenyl)-4,4-dimethylcyclopenten-1-yl]benzenesulfonamide (D-181);
4-[2-(4-fluorophenyl)cyclopenten-1-yl]benzenesulfonamide (D-182);
4-[2-(4-chlorophenyl)cyclopenten-1-yl]benzenesulfonamide (D-183);
1-[2-(4-methoxyphenyl)cyclopenten-1-yl]-4-(methylsulfonyl)benzene (D-184);
1-[2-(2,3-difluorophenyl)cyclopenten-1-yl]-4-(methylsulfonyl)benzene (D-185);
4-[2-(3-fluoro-4-methoxyphenyl)cyclopenten-1-yl]benzenesulfonamide (D-186);
1-[2-(3-chloro-4-methoxyphenyl)cyclopenten-1-yl]-4-(methylsulfonyl)benzene (D-187);
4-[2-(3-chloro-4-fluorophenyl)cyclopenten-1-yl]benzenesulfonamide (D-188);
4-[2-(2-methylpyridin-5-yl)cyclopenten-1-yl]benzenesulfonamide (D-189);
ethyl 2-[4-(4-fluorophenyl)-5-[4-(methylsulfonyl)phenyl]oxazol-2-yl]-2-benzyl-acetate (D-190);
2-[4-(4-fluorophenyl)-5-[4-(methylsulfonyl)phenyl]oxazol-2-yl]acetic acid (D-191);
2-(tert-butyl)-4-(4-fluorophenyl)-5-[4-(methylsulfonyl)phenyl]oxazole (D-192);
4-(4-fluorophenyl)-5-[4-(methylsulfonyl)phenyl]-2-phenyloxazole (D-193);
4-(4-fluorophenyl)-2-methyl-5-[4-(methylsulfonyl)phenyl]oxazole (D-194);
4-[5-(3-fluoro-4-methoxyphenyl)-2-trifluoromethyl-4-oxazolyl]benzenesulfonamide (D-195);
6-chloro-7-(1,1-dimethylethyl)-2-trifluoromethyl-2H-1-benzopyran-3-carboxylic acid (D-196);
6-chloro-8-methyl-2-trifluoromethyl-2h-1-benzopyran-3-carboxylic acid (D-197);
5,5-dimethyl-3-(3-fluorophenyl)-4-(4-methyl-sulphonyl-2(5H)-fluranone (D-198);
6-chloro-2-trifluoromethyl-2H-1-benzothiopyran-3-carboxylic acid (D-199);
4-[5-(4-chlorophenyl)-3-(trifluoromethyl)-1H-pyrazol-1-yl]benzenesulfonamide (D-200);
4-[5-(4-methylphenyl)-3-(trifluoromethyl)-1H-pyrazol-1-yl]benzenesulfonamide (D-201);
4-[5-(3-fluoro-4-methoxyphenyl)-3-(difluoromethyl)-1H-pyrazol-1-yl]benzenesulfonamide (D-202);
3-[1-[4-(methylsulfonyl)phenyl]-4-trifluoromethyl-1H-imidazol-2-yl]pyridine (D-203);
2-methyl-5-[1-[4-(methylsulfonyl)phenyl]-4-trifluoromethyl-1H-imidazol-2-yl]pyridine (D-204);
4-[2-(5-methylpyridin-3-yl)-4-(trifluoromethyl)-1H-imidazol-1-yl]benzenesulfonamide (D-205);
4-[5-methyl-3-phenylisoxazol-4-yl]benzenesulfonamide (D-206);
4-[5-hydroxymethyl-3-phenylisoxazol-4-yl]benzenesulfonamide (D-207);
[2-trifluoromethyl-5-(3,4-difluorophenyl)-4-oxazolyl]benzenesulfonamide (D-208);
4-[2-methyl-4-phenyl-5-oxazolyl]benzenesulfonamide (D-209);
4-[5-(3-fluoro-4-methoxyphenyl-2-trifluoromethyl)-4-oxazolyl]benzenesulfonamide (D-210);
[2-(2-Chloro-6-fluoro-phenylamino)-5-methyl-phenyl]-acetic acid, COX 189 (D-211);
N-(4-nitro-2-phenoxy-phenyl)methanesulfonamide, Nimesulide (D-212);
N-[6-(2,4-Difluoro-phenoxy)-1-oxo-indan-5-yl]-methanesulfonamide, Flosulide (D-213);
N-[6-(2,4-difluoro-phenylsulfonyl)-1-1-oxo-1H-inden-5-yl]-methanesulfonmaide, sodium salt, or L-745337 (D-214);
N-[5,(4-fluoro-phenylsulfanyl)-thiophen-2-yl]methanesulfonamide or RWJ-63556 (D-215);
(5Z)-2-amino-5-[[3,5-bis(1,1-dimethylethyl)-4-hydroxyphenyl]methylene]-4(5H)-thiazolone, Darbufelone (D-217);
N-[3-(formylamino)-4-oxo-6-phenoxy-4H-1-benzopyran-7-yl]-methanesulfonamide, T-614 (D-224);
(6aR,10aR)-3-(1,1-dimethylheptyl)-6a,7,10,10a-tetrahydro-1-hydroxy-6,6-dimethyl-6H-dibenzo[b,d]pyran-9-carboxylic acid, CT3 (D-227);
4-[[3,5-bis(1,1-dimethylethyl)-4-hydroxyphenyl]methylene]dihydro-2-methyl-2H-1,2-oxazin-3(4H)-one, BF-389 (D-229); or
6-dioxo-9H-purin-8-yl-cinnamic acid (D-231);
or a pharmaceutically acceptable salt or derivative or prodrug thereof.

8. The method of claim 7 wherein the cyclooxygenase-2 selective inhibitor is D-1 to D-5, D-6 to D-10, D-11 to D-15, D-16 to D-20, D-21 to D-25, D-26 to D-30, D-31 to D-35, D-36 to D-40, D-41 to D-45, D-46 to D-50, D-51 to D-55, D-56 to D-60, D-61 to D-65, D-66 to D-70, D-71 to D-75, D-76 to D-80, D-81 to D-85, D-86 to D-90, D-91 to D-95, D-96 to D-100, D-101 to D-105, D-106 to D-110, D-111 to D-115, D-116 to D-120, D-121 to D-125, D-126 to D-130, D-131 to D-135, D-136 to D-140, D-141 to D-145, D-146 to D-150, D-151 to D-155, D-156 to D-160, D-161 to D-165, D-166 to D-170, D-171 to D-175, D-176 to D-180, D-181 to D-185, D-186 to D-190, D-191 to D-195, D-196 to D-200, D-201 to D-205, D-206 to D-210, D-211 to D-215, D-217, D-224, D-227, D-229, D-231, or a pharmaceutically acceptable salt or derivative or prodrug thereof.

9. The method of claim 1 further comprising treating the subject with an amount of an HMG-CoA reductase inhibitor wherein the amount of the apical sodium co-dependent bile acid transport inhibitor and the amount of the cyclooxygenase-2 selective inhibitor and the amount of the HMG-CoA reductase inhibitor together constitute a hypercholesterolemia-related condition effective amount or an inflammation-related condition effective amount of the apical sodium co-dependent bile acid transport inhibitor, the cyclooxygenase-2 selective inhibitor and the HMG-CoA reductase inhibitor.

10. The method of claim 9 wherein the HMG-CoA reductase inhibitor is selected from the group consisting of fluvastatin, lovastatin, pravastatin, simvastatin, atorvastatin, cerivastatin, bervastatin, rosuvastatin, and itavastatin, or a pharmaceutically acceptable salt or ester or lactone thereof.

11. A pharmaceutical combination comprising an amount of an apical sodium co-dependent bile acid transport inhibitor, an amount of a cyclooxygenase-2 selective inhibitor or prodrug, and a pharmaceutically acceptable carrier, wherein the amount of the apical sodium co-dependent bile acid transport inhibitor and the amount of the cyclooxygenase-2 selective inhibitor together constitute a hypercholesterolemia-related condition effective amount or an inflammation-related condition effective amount of the apical sodium co-dependent bile acid transport inhibitor and the cyclooxygenase-2 selective inhibitor.

12. The combination of claim 11 wherein the cyclooxygenase-2 selective inhibitor is
[2-(2,4-Dichloro-6-ethyl-3,5-dimethyl-phenylamino)-5-propyl-phenyl]-acetic acid (D-1);
6-[[5-(4-chlorobenzoyl)-1,4-dimethyl-1H-pyrrol-2-yl]methyl]-3(2H)-pyridazinone or RS 57067 (D-2);
6-Nitro-2-trifluoromethyl-2H-1-benzopyran-3-carboxylic acid (D-3);
6-Chloro-8-methyl-2-trifluoromethyl-2H-1-benzopyran-3-carboxylic acid (D-4);
((S)-6-chloro-7-(1,1-dimethylethyl)-2-trifluoromethyl-2H-1-benzopyran-3-carboxylic acid (D-5);
2-Trifluoromethyl-2H-naphtho[2,3-b]pyran-3-carboxylic acid (D-6);
6-Chloro-7-(4-nitrophenoxy)-2-(trifluoromethyl)-2H-1-benzopyran-3-carboxylic acid (D-7);
((S)-6,8-Dichloro-2-(trifluoromethyl)-2H-1-benzopyran-3-carboxylic acid (D-8);
6-Chloro-2-(trifluoromethyl)-4-phenyl-2H-1-benzopyran-3-carboxylic acid (D-9);
6-(4-Hydroxybenzoyl)-2-(trifluoromethyl)-2H-1-benzopyran-3-carboxylic acid (D-10);
2-(Trifluoromethyl)-6-[(trifluoromethyl)thio]-2H-1-benzothiopyran-3-carboxylic acid (D-11);
6,8-Dichloro-2-trifluoromethyl-2H-1-benzothiopyran-3-carboxylic acid (D-12);
6-(1,1-Dimethylethyl)-2-(trifluoromethyl)-2H-1-benzothiopyran-3-carboxylic acid (D-13);
6,7-Difluoro-1,2-dihydro-2-(trifluoromethyl)-3-quinolinecarboxylic acid (D-14);
6-Chloro-1,2-dihydro-1-methyl-2-(trifluoromethyl)-3-quinolinecarboxylic acid (D-15);
6-Chloro-2-(trifluoromethyl)-1,2-dihydro[1,8]naphthyridine-3-carboxylic acid (D-16);
((S)-6-Chloro-1,2-dihydro-2-(trifluoromethyl)-3-quinolinecarboxylic acid (D-17);
celecoxib (D-18);
valdecoxib (D-19);
deracoxib (D-20);
rofecoxib (D-21);
etoricoxib (D-22);
JTE-522 (D-23);
parecoxib (D-24)
ABT-963 (D-25);
N-(2-cyclohexyloxy-4-nitro-phenyl)-methanesulfonamide or NS-398 (D-26);
6-chloro-2-trifluoromethyl-2H-1-benzopyran-3-carboxylic acid (D-27);
6-chloro-7-methyl-2-trifluoromethyl-2H-1-benzopyran-3-carboxylic acid (D-28);
8-(1-methylethyl)-2-trifluoromethyl-2H-1-benzopyran-3-carboxylic acid (D-29);
6-chloro-8-(1-methylethyl)-2-trifluoromethyl-2H-1-benzopyran-3-carboxylic acid (D-30);
2-trifluoromethyl-3H-naphthopyran-3-carboxylic acid (D-31);
7-(1,1-dimethylethyl)-2-trifluoromethyl-2H-1-benzopyran-3-carboxylic acid (D-32);
6-bromo-2-trifluoromethyl-2H-1-benzopyran-3-carboxylic acid (D-33);
8-chloro-2-trifluoromethyl-2H-1-benzopyran-3-carboxylic acid (D-34);
6-trifluoromethoxy-2-trifluoromethyl-2H-1-benzopyran-3-carboxylic acid (D-35);
5,7-dichloro-2-trifluoromethyl-2H-1-benzopyran-3-carboxylic acid (D-36);
8-phenyl-2-trifluoromethyl-2H-1-benzopyran-3-carboxylic acid (D-37);
7,8-dimethyl-2-trifluoromethyl-2H-1-benzopyran-3-carboxylic acid (D-38);
6,8-bis(dimethylethyl)-2-trifluoromethyl-2H-1-benzopyran-3-carboxylic acid (D-39);
7-(1-methylethyl)-2-trifluoromethyl-2H-1-benzopyran-3-carboxylic acid (D-40);
7-phenyl-2-trifluoromethyl-2H-1-benzopyran-3-carboxylic acid (D-41);
6-chloro-7-ethyl-2-trifluoromethyl-2H-1-benzopyran-3-carboxylic acid (D-42);
6-chloro-8-ethyl-2-trifluoromethyl-2H-1-benzopyran-3-carboxylic acid (D-43);
6-chloro-7-phenyl-2-trifluoromethyl-2H-1-benzopyran-3-carboxylic acid (D-44);
6,7-dichloro-2-trifluoromethyl-2H-1-benzopyran-3-carboxylic acid (D-45);
6,8-dichloro-2-trifluoromethyl-2H-1-benzopyran-3-carboxylic acid (D-46);
2-trifluoromethyl-3H-naptho[2,1-b]pyran-3-carboxylic acid (D-29);
8-chloro-6-methyl-2-trifluoromethyl-2H-1-benzopyran-3-carboxylic acid (D-48
8-chloro-6-methoxy-2-trifluoromethyl-2H-1-benzopyran-3-carboxylic acid (D-49);
6-bromo-8-chloro-2-trifluoromethyl-2H-1-benzopyran-3-carboxylic acid (D-50);
8-bromo-6-fluoro-2-trifluoromethyl-2H-1-benzopyran-3-carboxylic acid (D-51);
8-bromo-6-methyl-2-trifluoromethyl-2H-1-benzopyran-3-carboxylic acid (D-52);
8-bromo-5-fluoro-2-trifluoromethyl-2H-1-benzopyran-3-carboxylic acid (D-53);
6-chloro-8-fluoro-2-trifluoromethyl-2H-1-benzopyran-3-carboxylic acid (D-54);
6-bromo-8-methoxy-2-trifluoromethyl-2H-1-benzopyran-3-carboxylic acid (D-55);
6-[[(phenylmethyl)amino]sulfonyl]-2-trifluoromethyl-2H-1-benzopyran-3-carboxylic acid (D-56);
6-[(dimethylamino)sulfonyl]-2-trifluoromethyl-2H-1-benzopyran-3-carboxylic acid (D-57);
6-[(methylamino)sulfonyl]-2-trifluoromethyl-2H-1-benzopyran-3-carboxylic acid (D-58);
6-[(4-morpholino)sulfonyl]-2-trifluoromethyl-2H-1-benzopyran-3-carboxylic acid (D-59);
6-[(1,1-dimethylethyl)aminosulfonyl]-2-trifluoromethyl-2H-1-benzopyran-3-carboxylic acid (D-60);
6-[(2-methylpropyl)aminosulfonyl]-2-trifluoromethyl-2H-1-benzopyran-3-carboxylic acid (D-61);
6-methylsulfonyl-2-trifluoromethyl-2H-1-benzopyran-3-carboxylic acid (D-62);
8-chloro-6-[[(phenylmethyl)amino]sulfonyl]-2-trifluoromethyl-2H-1-benzopyran-3-carboxylic acid (D-63);
6-phenylacetyl-2-trifluoromethyl-2H-1-benzopyran-3-carboxylic acid (D-64);
6,8-dibromo-2-trifluoromethyl-2H-1-benzopyran-3-carboxylic acid (D-65);
8-chloro-5,6-dimethyl-2-trifluoromethyl-2H-1-benzopyran-3-carboxylic acid (D-66);
6,8-dichloro-(S)-2-trifluoromethyl-2H-1-benzopyran-3-carboxylic acid (D-67);
6-benzylsulfonyl-2-trifluoromethyl-2H-1-benzopyran-3-carboxylic acid (D-68);
6-[[N-(2-furylmethyl)amino]sulfonyl]-2-trifluoromethyl-2H-1-benzopyran-3-carboxylic acid (D-69);
6-[[N-(2-phenylethyl)aminolsulfonyl]-2-trifluoromethyl-2H-1-benzopyran-3-carboxylic acid (D-70);
6-iodo-2-trifluoromethyl-2H-1-benzopyran-3-carboxylic acid (D-71);
7-(1,1-dimethylethyl)-2-pentafluoroethyl-2H-1-benzopyran-3-carboxylic acid (D-72);
6-chloro-2-trifluoromethyl-2H-1-benzothiopyran-3-carboxylic acid (D-73); BMS-347070 (D-74);
8-acetyl-3-(4-fluorophenyl)-2-(4-methylsulfonyl)phenyl-imidazo(1,2-a)pyridine (D-75);
5,5-dimethyl-4-(4-methylsulfonyl)phenyl-3-phenyl-2-(5H)-furanone (D-76);
5-(4-fluorophenyl)-1-[4-(methylsulfonyl)phenyl]-3-(trifluoromethyl)pyrazole (D-77);
4-(4-fluorophenyl)-5-[4-(methylsulfonyl)phenyl]-1-phenyl-3-(trifluoromethyl)pyrazole (D-78);
4-(5-(4-chlorophenyl)-3-(4-methoxyphenyl)-1H-pyrazol-1-yl)benzenesulfonamide(D-79);
4-(3,5-bis(4-methylphenyl)-1H-pyrazol-1-yl)benzenesulfonamide(D-80);
4-(5-(4-chlorophenyl)-3-phenyl-1H-pyrazol-1-yl)benzenesulfonamide(D-81);
4-(3,5-bis(4-methoxyphenyl)-1H-pyrazol-1-yl)benzenesulfonamide(D-82);
4-(5-(4-chlorophenyl)-3-(4-methylphenyl)-1H-pyrazol-1-yl)benzenesulfonamide(D-83);
4-(5-(4-chlorophenyl)-3-(4-nitrophenyl)-1H-pyrazol-1-yl)benzenesulfonamide(D-84);
4-(5-(4-chlorophenyl)-3-(5-chloro-2-thienyl)-1H-pyrazol-1-yl)benzenesulfonamide(D-85);
4-(4-chloro-3,5-diphenyl-1H-pyrazol-1-yl)benzenesulfonamide(D-86);
4-[5-(4-chlorophenyl)-3-(trifluoromethyl)-1H-pyrazol-1-yl]benzenesulfonamide(D-87);
4-[5-phenyl-3-(trifluoromethyl)-1H-pyrazol-1-yl]benzenesulfonamide (D-88);
4-[5-(4-fluorophenyl)-3-(trifluoromethyl)-1H-pyrazol-1-yl]benzenesulfonamide (D-89);
4-[5-(4-methoxyphenyl)-3-(trifluoromethyl)-1H-pyrazol-1-yl]benzenesulfonamide (D-90);
4-[5-(4-chlorophenyl)-3-(difluoromethyl)-1H-pyrazol-1-yl]benzenesulfonamide (D-91);
4-[5-(4-methylphenyl)-3-(trifluoromethyl)-1H-pyrazol-1-yl]benzenesulfonamide (D-92);
4-[4-chloro-5-(4-chlorophenyl)-3-(trifluoromethyl)-1H-pyrazol-1-yl]benzenesulfonamide (D-93);
4-[3-(difluoromethyl)-5-(4-methylphenyl)-1H-pyrazol-1-yl]benzenesulfonamide (D-94);
4-[3-(difluoromethyl)-5-phenyl-1H-pyrazol-1-yl]benzenesulfonamide (D-95);
4-[3-(difluoromethyl)-5-(4-methoxyphenyl)-1H-pyrazol-1-yl]benzenesulfonamide (D-96);
4-[3-cyano-5-(4-fluorophenyl)-1H-pyrazol-1-yl]benzenesulfonamide (D-97);
4-[3-(difluoromethyl)-5-(3-fluoro-4-methoxyphenyl)-1H-pyrazol-1-yl]benzenesulfonamide (D-98);
4-[5-(3-fluoro-4-methoxyphenyl)-3-(trifluoromethyl)-1H-pyrazol-1-yl]benzenesulfonamide (D-99);
4-[4-chloro-5-phenyl-1-H-pyrazol-1-yl]benzenesulfonamide (D-100);
4-[5-(4-chlorophenyl)-3-(hydroxymethyl)-1H-pyrazol-1-yl]benzenesulfonamide (D-101);
4-[5-(4-(N,N-dimethylamino)phenyl)-3-(trifluoromethyl)-1H-pyrazol-1-yl]benzenesulfonamide (D-102);
5-(4-fluorophenyl)-6-[4-(methylsulfonyl)phenyl]spiro[2.4]hept-5-ene (D-103);
4-[6-(4-fluorophenyl)spiro[2.4]hept-5-en-5-yl]benzenesulfonamide (D-104);
6-(4-fluorophenyl)-7-[4-(methylsulfonyl)phenyl]spiro[3.4]oct-6-ene (D-105);
5-(3-chloro-4-methoxyphenyl)-6-[4-(methylsulfonyl)phenyl]spiro[2.4]hept-5-ene (D-106);
4-[6-(3-chloro-4-methoxyphenyl)spiro[2.4]hept-5-en-5-yl]benzenesulfonamide (D-107);
5-(3,5-dichloro-4-methoxyphenyl)-6-[4-(methylsulfonyl)phenyl]spiro[2.4]hept-5-ene (D-108);
5-(3-chloro-4-fluorophenyl)-6-[4-(methylsulfonyl)phenyl]spiro[2.4]hept-5-ene (D-109);
4-[6-(3,4-dichlorophenyl)spiro[2.4]hept-5-en-5-yl]benzenesulfonamide (D-110);
2-(3-chloro-4-fluorophenyl)-4-(4-fluorophenyl)-5-(4-methylsulfonylphenyl)thiazole (D-111);
2-(2-chlorophenyl)-4-(4-fluorophenyl)-5-(4-methylsulfonylphenyl)thiazole (D-112);
5-(4-fluorophenyl)-4-(4-methylsulfonylphenyl)-2-methylthiazole (D-113);
4-(4-fluorophenyl)-5-(4-methylsulfonylphenyl)-2-trifluoromethylthiazole (D-114);
4-(4-fluorophenyl)-5-(4-methylsulfonylphenyl)-2-(2-thienyl)thiazole (D-115);
4-(4-fluorophenyl)-5-(4-methylsulfonylphenyl)-2-benzylaminothiazole (D-116);
4-(4-fluorophenyl)-5-(4-methylsulfonylphenyl)-2-(1-propylamino)thiazole (D-117);
2-[(3,5-dichlorophenoxy)methyl)-4-(4-fluorophenyl)-5-[4-(methylsulfonyl)phenyl]thiazole (D-118);
5-(4-fluorophenyl)-4-(4-methylsulfonylphenyl)-2-trifluoromethylthiazole (D-119);
1-methylsulfonyl-4-[1,1-dimethyl-4-(4-fluorophenyl)cyclopenta-2,4-dien-3-yl]benzene (D-120);
4-[4-(4-fluorophenyl)-1,1-dimethylcyclopenta-2,4-dien-3-yl]benzenesulfonamide (D-121);
5-(4-fluorophenyl)-6-[4-(methylsulfonyl)phenyl]spiro[2.4]hepta-4,6-diene (D-122);
4-[6-(4-fluorophenyl)spiro[2.4]hepta-4,6-dien-5-yl]benzenesulfonamide (D-123);
6-(4-fluorophenyl)-2-methoxy-5-[4-(methylsulfonyl)phenyl]-pyridine-3-carbonitrile (D-124);
2-bromo-6-(4-fluorophenyl)-5-[4-(methylsulfonyl)phenyl]-pyridine-3-carbonitrile (D-125);
6-(4-fluorophenyl)-5-[4-(methylsulfonyl)phenyl]-2-phenyl-pyridine-3-carbonitrile (D-126);
4-[2-(4-methylpyridin-2-yl)-4-(trifluoromethyl)-1H-imidazol-1-yl]benzenesulfonamide (D-127);
4-[2-(5-methylpyridin-3-yl)-4-(trifluoromethyl)-1H-imidazol-1-yl]benzenesulfonamide (D-128);
4-[2-(2-methylpyridin-3-yl)-4-(trifluoromethyl)-1H-imidazol-1-yl]benzenesulfonamide (D-129);
3-[1-[4-(methylsulfonyl)phenyl]-4-(trifluoromethyl)-1H-imidazol-2-yl]pyridine (D-130);
2-[1-[4-(methylsulfonyl)phenyl-4-(trifluoromethyl)-1H-imidazol-2-yl]pyridine (D-131);
2-methyl-4-[1-[4-(methylsulfonyl)phenyl-4-(trifluoromethyl)-1H-imidazol-2-yl]pyridine (D-132);
2-methyl-6-[1-[4-(methylsulfonyl)phenyl-4-(trifluoromethyl)-1H-imidazol-2-yl]pyridine (D-133);
4-[2-(6-methylpyridin-3-yl)-4-(trifluoromethyl)-1H-imidazol-1-yl]benzenesulfonamide (D-134);
2-(3,4-difluorophenyl)-1-[4-(methylsulfonyl)phenyl]-4-(trifluoromethyl)-1H-imidazole (D-135);
4-[2-(4-methylphenyl)-4-(trifluoromethyl)-1H-imidazol-1-yl]benzenesulfonamide (D-136);
2-(4-chlorophenyl)-1-[4-(methylsulfonyl)phenyl]-4-methyl-1H-imidazole (D-137);
2-(4-chlorophenyl)-1-[4-(methylsulfonyl)phenyl]-4-phenyl-1H-imidazole (D-138);
2-(4-chlorophenyl)-4-(4-fluorophenyl)-1-[4-(methylsulfonyl)phenyl]1-imidazole (D-139);
2-(3-fluoro-4-methoxyphenyl)-1-[4-(methylsulfonyl)phenyl-4-(trifluoromethyl)-1H-imidazole (D-140);
1-[4-(methylsulfonyl)phenyl]-2-phenyl-4-trifluoromethyl-1H-imidazole (D-141);
2-(4-methylphenyl)-1-[4-(methylsulfonyl)phenyl]-4-trifluoromethyl-1H-imidazole (D-142);
4-[2-(3-chloro-4-methylphenyl)-4-(trifluoromethyl)-1H-imidazol-1-yl]benzenesulfonamide (D-143);
2-(3-fluoro-5-methylphenyl)-1-[4-(methylsulfonyl)phenyl]-4-(trifluoromethyl)-1H-imidazole (D-144);
4-[2-(3-fluoro-5-methylphenyl)-4-(trifluoromethyl)-1H-imidazol-1-yl]benzenesulfonamide (D-145);
2-(3-methylphenyl)-1-[4-(methylsulfonyl)phenyl]-4-trifluoromethyl-1H-imidazole (D-146);
4-[2-(3-methylphenyl)-4-trifluoromethyl-1H-imidazol-1-yl]benzenesulfonamide (D-147);
1-[4-(methylsulfonyl)phenyl]-2-(3-chlorophenyl)-4-trifluoromethyl-1H-imidazole (D-148);
4-[2-(3-chlorophenyl)-4-trifluoromethyl-1H-imidazol-1-yl]benzenesulfonamide (D-149);
4-[2-phenyl-4-trifluoromethyl-1H-imidazol-1-yl]benzenesulfonamide (D-150);
4-[2-(4-methoxy-3-chlorophenyl)-4-trifluoromethyl-1H-imidazol-1-yl]benzenesulfonamide (D-151);
1-allyl-4-(4-fluorophenyl)-3-[4-(methylsulfonyl)phenyl]-5-(trifluoromethyl)-1H-pyrazole (D-152);
4-[1-ethyl-4-(4-fluorophenyl)-5-(trifluoromethyl)-1H-pyrazol-3-yl]benzenesulfonamide (D-153);
N-phenyl-[4-(4-fluorophenyl)-3-[4-(methylsulfonyl)phenyl]-5-(trifluoromethyl)-1H-pyrazol-1-yl]acetamide (D-154);
ethyl [4-(4-fluorophenyl)-3-[4-(methylsulfonyl)phenyl]-5-(trifluoromethyl)-1H-pyrazol-1-yl]acetate (D-155);
4-(4-fluorophenyl)-3-[4-(methylsulfonyl)phenyl]-1-(2-phenylethyl)-1H-pyrazole (D-156);
4-(4-fluorophenyl)-3-[4-(methylsulfonyl)phenyl]-1-(2-phenylethyl)-5-(trifluoromethyl)pyrazole (D-157);
1-ethyl-4-(4-fluorophenyl)-3-[4-(methylsulfonyl)phenyl]-5-(trifluoromethyl)-1H-pyrazole (D-158);
5-(4-fluorophenyl)-4-(4-methylsulfonylphenyl)-2-trifluoromethyl-1H-imidazole (D-159);
4-[4-(methylsulfonyl)phenyl]-5-(2-thiophenyl)-2-(trifluoromethyl)-1H-imidazole (D-160);
5-(4-fluorophenyl)-2-methoxy-4-[4-(methylsulfonyl)phenyl]-6-(trifluoromethyl)pyridine (D-161);
2-ethoxy-5-(4-fluorophenyl)-4-[4-(methylsulfonyl)phenyl]-6-(trifluoromethyl)pyridine (D-162);
5-(4-fluorophenyl)-4-[4-(methylsulfonyl)phenyl]-2-(2-propynyloxy)-6-(trifluoromethyl)pyridine (D-163);
2-bromo-5-(4-fluorophenyl)-4-[4-(methylsulfonyl)phenyl]-6-(trifluoromethyl)pyridine (D-164);
4-[2-(3-chloro-4-methoxyphenyl)-4,5-difluorophenyl]benzenesulfonamide (D-165);
1-(4-fluorophenyl)-2-[4-(methylsulfonyl)phenyl]benzene (D-166);
5-difluoromethyl-4-(4-methylsulfonylphenyl)-3-phenylisoxazole (D-167);
4-[3-ethyl-5-phenylisoxazol-4-yl]benzenesulfonamide (D-168);
4-[5-difluoromethyl-3-phenylisoxazol-4-yl]benzenesulfonamide (D-169);
4-[5-hydroxymethyl-3-phenylisoxazol-4-yl]benzenesulfonamide (D-170);
4-[⁵-methyl-3-phenyl-isoxazol-4-yl]benzenesulfonamide (D-171);
1-[2-(4-fluorophenyl)cyclopenten-1-yl]-4-(methylsulfonyl)benzene (D-172);
1-[2-(4-fluoro-2-methylphenyl)cyclopenten-1-yl]-4-(methylsulfonyl)benzene (D-173);
1-[2-(4-chlorophenyl)cyclopenten-1-yl]-4-(methylsulfonyl)benzene (D-174);
1-[2-(2,4-dichlorophenyl)cyclopenten-1-yl]-4-(methylsulfonyl)benzene (D-175);
1-[2-(4-trifluoromethylphenyl)cyclopenten-1-yl]-4-(methylsulfonyl)benzene (D-176);
1-[2-(4-methylthiophenyl) cyclopenten-yl]-4-(methylsulfonyl)benzene (D-177);
1-[2-(4-fluorophenyl)-4,4-dimethylcyclopenten-1-yl]-4-(methylsulfonyl)benzene (D-178);
4-[2-(4-fluorophenyl)-4,4-dimethylcyclopenten-i-yl]benzenesulfonamide (D-179);
1-[2-(4-chlorophenyl)-4,4-dimethylcyclopenten-1-yl]-4-(methylsulfonyl)benzene (D-180);
4-[2-(4-chlorophenyl)-4,4-dimethylcyclopenten-1-yl]benzenesulfonamide (D-181);
4-[2-(4-fluorophenyl)cyclopenten-1-yl]benzenesulfonamide (D-182);
4-[2-(4-chlorophenyl)cyclopenten-1-yl]benzenesulfonamide (D-183);
1-[2-(4-methoxyphenyl)cyclopenten-1-yl -4-(methylsulfonyl)benzene (D-184);
1-[2-(2, 3-difluorophenyl)cyclopenten-1-yl -4-(methylsulfonyl)benzene (D-185);
4-[2-(3-f luoro-4-methoxyphenyl) cyclopenten-1-yl]benzenesulfonamide (D-186);
1-[2-(3-chloro-4-methoxyphenyl) cyclopenten-1-yl]-4-(methylsulfonyl)benzene (D-187);
4-[2-(3-chloro-4-fluorophenyl) cyclopenten-1-yl]benzenesulfonamide (D-188);
4-[2-(2-methylpyridin-5-yl)cyclopenten-1-yl]benzenesulfonamide (D-189);
ethyl 2-[4-(4-fluorophenyl)-5-[4-(methylsulfonyl)phenyl]oxazol-2-yl]-2-benzyl-acetate (D-190);
2-[4-(4-fluorophenyl)-5-[4-(methylsulfonyl)phenyl]oxazol-2-yl]acetic acid (D-191);
2-(tert-butyl)-4-(4-fluorophenyl)-5-[4-(methylsulfonyl)phenyl]oxazole (D-192);
4-(4-fluorophenyl)-5-[4-(methylsulfonyl)phenyl)-2-phenyloxazole (D-193);
4-(4-fluorophenyl)-2-methyl-5-f4-(methylsulfonyl)phenyl]oxazole (D-194);
4-[5-(3-fluoro-4-methoxyphenyl)-2-trifluoromethyl-4-oxazolyl]benzenesulfonamide (D-195);
6-chloro-7-(1,1-dimethylethyl)-2-trifluoromethyl-2H-1-benzopyran-3-carboxylic acid (D-196);
6-chloro-8-methyl-2-trifluoromethyl-2h-1-benzopyran-3-carboxylic acid (D-197);
5,5-dimethyl-3-(3-fluorophenyl)-4-(4-methyl-sulphonyl-2(5H)-fluranone (D-198);
6-chloro-2-trifluoromethyl-2H-1-benzothiopyran-3-carboxylic acid (D-199);
4-[5-(4-chlorophenyl)-3-(trifluoromethyl)-1H-pyrazol-1-yl]benzenesulfonamide (D-200);
4-[5-(4-methylphenyl)-3-(trifluoromethyl)-1H-pyrazol-1-yl]benzenesulfonamide (D-201);
4-[5-(3-fluoro-4-methoxyphenyl)-3-(difluoromethyl)-1H-pyrazol-1-yl]benzenesulfonamide (D-202);
3-[1-[4-(methylsulfonyl)phenyl]-4-trifluoromethyl-1H-imidazol-2-yl]pyridine (D-203);
2-methyl-5-[1-[4-(methylsulfonyl)phenyl]-4-trifluoromethyl-1H-imidazol-2-yl]pyridine (D-204);
4-[2-(5-methylpyridin-3-yl)-4-(trifluoromethyl)-1H-imidazol-1-yl]benzenesulfonamide (D-205);
4-[5-methyl-3-phenylisoxazol-4-yl]benzenesulfonamide (D-206);
4-[5-hydroxymethyl-3-phenylisoxazol-4-yl]benzenesulfonamide (D-207);
[2-trifluoromethyl-5-(3,4-difluorophenyl)-4-oxazolyl]benzenesulfonamide (D-208);
4-[2-methyl-4-phenyl-5-oxazolyl]benzenesulfonamide (D-209);
4-[5-(3-fluoro-4-methoxyphenyl-2-trifluoromethyl)-4-oxazolyl)benzenesulfonamide (D-210);
[2-(2-Chloro-6-fluoro-phenylamino)-5-methyl-phenyl]-acetic acid, COX 189 (D-211);
N-(4-nitro-2-phenoxy-phenyl)methanesulfonamide, Nimesulide (D-212);
N-[6-(2,4-Difluoro-phenoxy)-1-oxo-indan-5-yl]-methanesulfonamide, Flosulide (D-213);
N-[6-(2,4-difluoro-phenylsulfonyl)-1-1-oxo-1H-inden-5-yl]-methanesulfonmaide, sodium salt, or L-745337 (D-214);
N-[5, (4-fluoro-phenylsulfanyl)-thiophen-2-yl]methanesulfonamide or RWJ-63556 (D-215);
(5Z)-2-amino-5-[[3,5-bis(1,1-dimethylethyl)-4-hydroxyphenyl]methylene]-4(5H)-thiazolone, Darbufelone (D-217);
N-[3-(formylamino)-4-oxo-6-phenoxy-4H-1-benzopyran-7-yl]-methanesulfonamide, T-614 (D-224);
(6aR,10aR)-3-(1,1-dimethylheptyl)-6a,7,10,10a-tetrahydro-1-hydroxy-6,6-dimethyl-6H-dibenzo[b,d]pyran-9-carboxylic acid, CT3 (D-227);
4-[[3,5-bis(1,1-dimethylethyl)-4-hydroxyphenyl]methylene]dihydro-2-methyl-2H-1,2-oxazin-3(4H)-one, BF-389 (D-229);
6-dioxo-9H-purin-8-yl-cinnamic acid (D-231);
or a pharmaceutically acceptable salt or derivative or prodrug thereof.

13. The combination of claim 11 wherein the cyclooxygenase-2 selective inhibitor is D-1 to D-5, D-6 to D-10, D-11 to D-15, D-16 to D-20, D-21 to D-25, D-26 to D-30, D-31 to D-35, D-36 to D-40, D-41 to D-45, D-46 to D-50, D-51 to D-55, D-56 to D-60, D-61 to D-65, D-66 to D-70, D-71 to D-75, D-76 to D-80, D-81 to D-85, D-86 to D-90, D-91 to D-95, D-96 to D-100, D-101 to D-105, D-106 to D-110, D-111 to D-115, D-116 to D-120, D-121 to D-125, D-126 to D-130, D-131 to D-135, D-136 to D-140, D-141 to D-145, D-146 to D-150, D-151 to D-155, D-156 to D-160, D-161 to D-165, D-166 to D-170, D-171 to D-175, D-176 to D-180, D-181 to D-185, D-186 to D-190, D-191 to D-195, D-196 to D-200, D-201 to D-205, D-206 to D-210, D-211 to D-215, D-217, D-224, D-227, D-229, D-231, or a pharmaceutically acceptable salt or derivative or prodrug thereof.

14. The combination of claim 11 further comprising an amount of an HMG-CoA reductase inhibitor wherein the amount of the apical sodium co-dependent bile acid transport inhibitor, the amount of the cyclooxygenase-2 selective inhibitor and the amount of the HMG-CoA reductase inhibitor together constitute a hypercholesterolemia-related condition effective amount or an inflammation-related condition effective amount of the apical sodium co-dependent bile acid transport inhibitor, the cyclooxygenase-2 selective inhibitor and the HMG-CoA reductase inhibitor.

15. The combination of claim 14 wherein the HMG-CoA reductase inhibitor is selected from the group consisting of fluvastatin, lovastatin, pravastatin, simvastatin, atorvastatin, cerivastatin, bervastatin, rosuvastatin, and itavastatin, or a pharmaceutically acceptable salt or ester or lactone thereof.

16. A kit comprised of an amount of an apical sodium co-dependent bile acid transport inhibitor in a dosage formulation and an amount of a cyclooxygenase-2 selective inhibitor or prodrug in a separate dosage formulation wherein the amount of the apical sodium co-dependent bile acid transport inhibitor and the amount of the cyclooxygenase-2 selective inhibitor together constitute a hypercholesterolemia-related condition effective amount or an inflammation-related condition effective amount of the apical sodium co-dependent bile acid transport inhibitor and the cyclooxygenase-2 selecti e inhibitor.

17. The kit of claim 16 wherein the cyclooxygenase-2 selective inhibitor is
[2-(2,4-Dichloro-6-ethyl-3,5-dimethyl-phenylamino)-5-propyl-phenyl]acetic acid (D-1);
6-[[5-(4-chlorobenzoyl)-1,4-dimethyl-1H-pyrrol-2-yl]methyl]-3(2H)-pyridazinone or RS 57067 (D-2);
6-Nitro-2-trifluoromethyl-2H-1-benzopyran-3-carboxylic acid (D-3);
6-Chloro-8-methyl-2-trifluoromethyl-2H-1-benzopyran-3-carboxylic acid (D-4);
((S)-6-chloro-7-(1,1-dimethylethyl)-2-trifluoromethyl-2H-1-benzopyran-3-carboxylic acid (D-5);
2-Trifluoromethyl-2H-naphtho[2,3-b]pyran-3-carboxylic acid (D-6);
6-Chloro-7-(4-nitrophenoxy)-2-(trifluoromethyl)-2H-1-benzopyran-3-carboxylic acid (D-7);
((S)-6,8-Dichloro-2-(trifluoromethyl)-2H-1-benzopyran-3-carboxylic acid (D-8);
6-Chloro-2-(trifluoromethyl)-4-phenyl-2H-1-benzopyran-3-carboxylic acid (D-9);
6-(4-Hydroxybenzoyl)-2-(trifluoromethyl)-2H-1-benzopyran-3-carboxylic acid (D-10);
2-(Trifluoromethyl)-6-[(trifluoromethyl)thio]-2H-1-benzothiopyran-3-carboxylic acid (D-11);
6,8-Dichloro-2-trifluoromethyl-2H-1-benzothiopyran-3-carboxylic acid (D-12);
6-(1,1-Dimethylethyl)-2-(trifluoromethyl)-2H-1-benzothiopyran-3-carboxylic acid (D-13);
6,7-Difluoro-1,2-dihydro-2-(trifluoromethyl)-3-quinolinecarboxylic acid (D-14);
6-Chloro-1,2-dihydro-1-methyl-2-(trifluoromethyl)-3-quinolinecarboxylic acid (D-15);
6-Chloro-2-(trifluoromethyl)-1,2-dihydro[1,8]naphthyridine-3-carboxylic acid (D-16);
((S)-6-Chloro-1,2-dihydro-2-(trifluoromethyl)-3-quinolinecarboxylic acid (D-17);
celecoxib (D-18);
valdecoxib (D-19);
deracoxib (D-20);
rofecoxib (D-21);
etoricoxib (D-22);
JTE-522 (D-23);
parecoxib (D-24)
ABT-963 (D-25);
N-(2-cyclohexyloxy-4-nitro-phenyl)-methanesulfonamide or NS-398 (D-26);
6-chloro-2-trifluoromethyl-2H-1-benzopyran-3-carboxylic acid (D-27);
6-chloro-7-methyl-2-trifluoromethyl-2H-1-benzopyran-3-carboxylic acid (D-28);
8-(1-methylethyl)-2-trifluoromethyl-2H-1-benzopyran-3-carboxylic acid (D-29);
6-chloro-8-(1-methylethyl)-2-trifluoromethyl-2H-1-benzopyran-3-carboxylic acid (D-30);
2-trifluoromethyl-3H-naphthopyran-3-carboxylic acid (D-31);
7-(1,1-dimethylethyl)-2-trifluoromethyl-2H-1-benzopyran-3-carboxylic acid (D-32);
6-bromo-2-trifluoromethyl-2H-1-benzopyran-3-carboxylic acid (D-33);
8-chloro-2-trifluoromethyl-2H-1-benzopyran-3-carboxylic acid (D-34);
6-trifluoromethoxy-2-trifluoromethyl-2H-1-benzopyran-3-carboxylic acid (D-35);
5,7-dichloro-2-trifluoromethyl-2H-1-benzopyran-3-carboxylic acid (D-36);
8-phenyl-2-trifluoromethyl-2H-1-benzopyran-3-carboxylic acid (D-37);
7,8-dimethyl-2-trifluoromethyl-2H-1-benzopyran-3-carboxylic acid (D-38);
6,8-bis(dimethylethyl)-2-trifluoromethyl-2H-1-benzopyran-3-carboxylic acid (D-39);
7-(1-methylethyl)-2-trifluoromethyl-2H-1-benzopyran-3-carboxylic acid (D-40);
7-phenyl-2-trifluoromethyl-2H-1-benzopyran-3-carboxylic acid (D-41);
6-chloro-7-ethyl-2-trifluoromethyl-2H-1-benzopyran-3-carboxylic acid (D-42);
6-chloro-8-ethyl-2-trifluoromethyl-2H-1-benzopyran-3-carboxylic acid (D-43);
6-chloro-7-phenyl-2-trifluoromethyl-2H-1-benzopyran-3-carboxylic acid (D-44);
6,7-dichloro-2-trifluoromethyl-2H-1-benzopyran-3-carboxylic acid (D-45);
6,8-dichloro-2-trifluoromethyl-2H-1-benzopyran-3-carboxylic acid (D-46);
2-trifluoromethyl-3H-naptho[2,1-blpyran-3-carboxylic acid (D-29);
8-chloro-6-methyl-2-trifluoromethyl-2H-1-benzopyran-3-carboxylic acid (D-48
8-chloro-6-methoxy-2-trifluoromethyl-2H-1-benzopyran-3-carboxylic acid (D-49);
6-bromo-8-chloro-2-trifluoromethyl-2H-1-benzopyran-3-carboxylic acid (D-50);
8-bromo-6-fluoro-2-trifluoromethyl-2H-1-benzopyran-3-carboxylic acid (D-51);
8-bromo-6-methyl-2-trifluoromethyl-2H-1-benzopyran-3-carboxylic acid (D-52);
8-bromo-5-fluoro-2-trifluoromethyl-2H-1-benzopyran-3-carboxylic acid (D-53);
6-chloro-8-fluoro-2-trifluoromethyl-2H-1-benzopyran-3-carboxylic acid (D-54);
6-bromo-8-methoxy-2-trifluoromethyl-2H-1-benzopyran-3-carboxylic acid (D-55);
6-[[((phenylmethyl)amino]sulfonyl]-2-trifluoromethyl-2H-1-benzopyran-3-carboxylic acid (D-56);
6-[(dimethylamino)sulfonyl]-2-trifluoromethyl-2H-1-benzopyran-3-carboxylic acid (D-57);
6-[(methylamino)sulfonyl]-2-trifluoromethyl-2H-1-benzopyran-3-carboxylic acid (D-58);
6-[(4-morpholino)sulfonyl]-2-trifluoromethyl-2H-1-benzopyran-3-carboxylic acid (D-59);
6-[(1,1-dimethylethyl)aminosulfonyl]-2-trifluoromethyl-2H-1-benzopyran-3-carboxylic acid (D-60);
6-[(2-methylpropyl)aminosulfonyl]-2-trifluoromethyl-2H-1-benzopyran-3-carboxylic acid (D-61);
6-methylsulfonyl-2-trifluoromethyl-2H-1-benzopyran-3-carboxylic acid (D-62);
8-chloro-6-[[(phenylmethyl)amino]sulfonyl]-2-trifluoromethyl-2H-1-benzopyran-3-carboxylic acid (D-63);
6-phenylacetyl-2-trifluoromethyl-2H-1-benzopyran-3-carboxylic acid (D-64);
6,8-dibromo-2-trifluoromethyl-2H-1-benzopyran-3-carboxylic acid (D-65);
8-chloro-5,6-dimethyl-2-trifluoromethyl-2H-1-benzopyran-3-carboxylic acid (D-66);
6,8-dichloro-(S)-2-trifluoromethyl-2H-1-benzopyran-3-carboxylic acid (D-67);
6-benzylsulfonyl-2-trifluoromethyl-2H-1-benzopyran-3-carboxylic acid (D-68);
6-[[N-(2-furylmethyl)amino]sulfonyl]-2-trifluoromethyl-2H-1-benzopyran-3-carboxylic acid (D-69);
6-[[N-(2-phenylethyl) amino]sulfonyl]-2-trifluoromethyl-2H-1-benzopyran-3-carboxylic acid (D-70);
6-iodo-2-trifluoromethyl-2H-1-benzopyran-3-carboxylic acid (D-71);
7-(1,1-dimethylethyl)-2-pentafluoroethyl-2H-1-benzopyran-3-carboxylic acid (D-72);
6-chloro-2-trifluoromethyl-2H-1-benzothiopyran-3-carboxylic acid (D-73);
BMS-347070 (D-74);
8-acetyl-3-(4-fluorophenyl)-2-(4-methylsulfonyl)phenyl-imidazo(1,2-a)pyridine (D-75);
5,5-dimethyl-4-(4-methylsulfonyl)phenyl-3-phenyl-2-(5H)-furanone (D-76);
5-(4-fluorophenyl)-1-[4-(methylsulfonyl)phenyl]-3-(trifluoromethyl)pyrazole (D-77);
4-(4-fluorophenyl)-5-[4-(methylsulfonyl)phenyl]-1-phenyl-3-(trifluoromethyl)pyrazole (D-78);
4-(5-(4-chlorophenyl)-3-(4-methoxyphenyl)-1H-pyrazol-1-yl)benzenesulfonamide(D-79);
4-(3,5-bis (4-methylphenyl)-1H-pyrazol-1-yl)benzenesulfonamide(D-80);
4-(5-(4-chlorophenyl)-3-phenyl-1H-pyrazol-1-yl)benzenesulfonamide (D-81);
4-(3, 5-bis(4-methoxyphenyl)-1H-pyrazol-1-yl)benzenesulfonamide(D-82);
4-(5-(4-chlorophenyl)-3-(4-methylphenyl)-1H-pyrazol-1-yl)benzenesulfonamide(D-83);
4-(5-(4-chlorophenyl)-3-(4-nitrophenyl)-1H-pyrazol-1-yl)benzenesulfonamide (D-84);
4-(5-(4-chlorophenyl)-3-(5-chloro-2-thienyl)-1H-pyrazol-1-yl)benzenesulfonamide(D-85);
4-(4-chloro-3,5-diphenyl-1H-pyrazol-1-yl)benzenesulfonamide(D-86);
4-[5-(4-chlorophenyl)-3-(trifluoromethyl)-1H-pyrazol-1-yl]benzenesulfonamide(D-87);
4-[5-phenyl-3-(trifluoromethyl)-1H-pyrazol-1-yl]benzenesulfonamide (D-88);
4-[5-(4-fluorophenyl)-3-(trifluoromethyl)-1H-pyrazol-1-yl]benzenesulfonamide (D-89);
4-(5-(4-methoxyphenyl)-3-(trifluoromethyl)-1H-pyrazol-1-yl]benzenesulfonamide (D-90);
4-[5-(4-chlorophenyl)-3-(difluoromethyl)-1H-pyrazol-1-yl]benzenesulfonamide (D-91);
4-[5-(4-methylphenyl)-3-(trifluoromethyl)-1H-pyrazol-1-yl]benzenesulfonamide (D-92);
4-[4-chloro-5-(4-chlorophenyl)-3-(trifluoromethyl)-1H-pyrazol-1-yl]benzenesulfonamide (D-93);
4-[3-(difluoromethyl)-5-(4-methylphenyl)-1H-pyrazol-1-yl]benzenesulfonamide (D-94);
4-[3-(difluoromethyl)-5-phenyl-1H-pyrazol-1-yl]benzenesulfonamide (D-95);
4-[3-(difluoromethyl)-5-(4-methoxyphenyl)-1H-pyrazol-1-yl]benzenesulfonamide (D-96);
4-[3-cyano-5-(4-fluorophenyl)-1H-pyrazol-1-yl]benzenesulfonamide (D-97);
4-[3-(difluoromethyl)-5-(3-fluoro-4-methoxyphenyl)-1H-pyrazol-1-yl]benzenesulfonamide (D-98);
4-[5-(3-fluoro-4-methoxyphenyl)-3-(trifluoromethyl)-1H-pyrazol-1-yl]benzenesulfonamide (D-99);
4-[4-chloro-5-phenyl-1H-pyrazol-1-yl]benzenesulfonamide (D-100);
4-[5-(4-chlorophenyl)-3-(hydroxymethyl)-1H-pyrazol-1-yl]benzenesulfonamide (D-101);
4-[5-(4-(N,N-dimethylamino)phenyl)-3-(trifluoromethyl)-1H-pyrazol-1-yl]benzenesulfonamide (D-102);
5-(4-fluorophenyl)-6-[4-(methylsulfonyl)phenyl]spiro[2.4]hept-5-ene (D-103);
4-[6-(4-fluorophenyl) spiro[2.4]hept-5-en-5-yl]benzenesulfonamide (D-104);
6-(4-fluorophenyl)-7-[4-(methylsulfonyl)phenyl]spiro[3.4]oct-6-ene (D-105);
5-(3-chloro-4-methoxyphenyl)-6-[4-(methylsulfonyl)phenyl]spiro[2.4]hept-5-ene (D-106);
4-[6-(3-chloro-4-methoxyphenyl)spiro[2.4]hept-5-en-5-yl]benzenesulfonamide (D-107);
5-(3,5-dichloro-4-methoxyphenyl)-6-[4-(methylsulfonyl)phenyl]spiro[2.4]hept-5-ene (D-108);
5-(3-chloro-4-fluorophenyl)-6-[4-(methylsulfonyl)phenyl]spiro[2.4]hept-5-ene (D-109);
4-[6-(3,4-dichlorophenyl)spiro[2.4]hept-5-en-5-yl]benzenesulfonamide (D-110);
2-(3-chloro-4-fluorophenyl)-4-(4-fluorophenyl)-5-(4-methylsulfonylphenyl)thiazole (D-111);
2-(2-chlorophenyl)-4-(4-fluorophenyl)-5-(4-methylsulfonylphenyl)thiazole (D-112);
5-(4-fluorophenyl)-4-(4-methylsulfonylphenyl)-2-methylthiazole (D-113);
4-(4-fluorophenyl)-5-(4-methylsulfonylphenyl)-2-trifluoromethylthiazole (D-114);
4-(4-fluorophenyl)-5-(4-methylsulfonylphenyl)-2-(2-thienyl)thiazole (D-115);
4-(4-f luorophenyl)-5-(4-methylsulfonylphenyl)-2-benzylaminothiazole (D-116);
4-(4-fluorophenyl)-5-(4-methylsulfonylphenyl)-2-(1-propylamino)thiazole (D-117);
2-[(3,5-dichlorophenoxy)methyl)-4-(4-fluorophenyl)-5-[4-(methylsulfonyl)phenyl]thiazole (D-118);
5-(4-fluorophenyl)-4-(4-methylsulfonylphenyl)-2-trifluoromethylthiazole (D-119);
1-methylsulfonyl-4-[1,1-dimethyl-4-(4-fluorophenyl)cyclopenta-2,4-dien-3-yl]benzene (D-120);
4-[4-(4-fluorophenyl)-1,1-dimethylcyclopenta-2,4-dien-3-yl)benzenesulfonamide (D-121);
5-(4-fluorophenyl)-6-[4-(methylsulfonyl)phenyl]spiro[2.4]hepta-4,6-diene (D-122);
4-[6-(4-fluorophenyl)spiro[2.4]hepta-4,6-dien-5-yl]benzenesulfonamide (D-123);
6-(4-fluorophenyl)-2-methoxy-5-[4-(methylsulfonyl)phenyl]-pyridine-3-carbonitrile (D-124);
2-bromo-6-(4-fluorophenyl)-5-[4-(methylsulfonyl)phenyl]-pyridine-3-carbonitrile (D-125);
6-(4-fluorophenyl)-5-[4-(methylsulfonyl)phenyl]-2-phenyl-pyridine-3-carbonitrile (D-126);
4-[2-(4-methylpyridin-2-yl)-4-(trifluoromethyl)-1H-imidazol-1-yl]benzenesulfonamide (D-127);
4-[2-(5-methylpyridin-3-yl)-4-(trifluoromethyl)-1H-imidazol-1-yl]benzenesulfonamide (D-128);
4-[2-(2-methylpyridin-3-yl)-4-(trifluoromethyl)-1H-imidazol-1-yl]benzenesulfonamide (D-129);
3-[1-[4-(methylsulfonyl)phenyl)-4-(trifluoromethyl)-1H-imidazol-2-yl]pyridine (D-130);
2-[1-[4-(methylsulfonyl)phenyl-4-(trifluoromethyl)-1H-imidazol-2-yl]pyridine (D-131);
2-methyl-4-[1-[4-(methylsulfonyl)phenyl-4-(trifluoromethyl)-1H-imidazol-2-yl]pyridine (D-132);
2-methyl-6-[1-[4-(methylsulfonyl)phenyl-4-(trifluoromethyl)-1H-imidazol-2-yl]pyridine (D-133);
4-[2-(6-methylpyridin-3-yl)-4-(trifluoromethyl)-1H-imidazol-1-yl]benzenesulfonamide (D-134);
2-(3,4-difluorophenyl)-1-[4-(methylsulfonyl)phenyl]-4-(trifluoromethyl)-1H-imidazole (D-135);
4-[2-(4-methylphenyl)-4-(trifluoromethyl)-1H-imidazol-1-yl]benzenesulfonamide (D-136);
2-(4-chlorophenyl)-1-[4-(methylsulfonyl)phenyl]-4-methyl-1H-imidazole (D-137);
2-(4-chlorophenyl)-1-[4-(methylsulfonyl)phenyl]-4-phenyl-1H-imidazole (D-138);
2-(4-chlorophenyl)-4-(4-fluorophehyl)-1-[4-(methylsulfonyl)phenyl]-1H-imidazole (D-139);
2-(3-fluoro-4-methoxyphenyl)-1-[4-(methylsulfonyl)phenyl-4-(trifluoromethyl)-1H-imidazole (D-140);
1-[4-(methylsulfonyl)phenyl]-2-phenyl-4-trifluoromethyl-1H-imidazole (D-141);
2-(4-methylphenyl)-1-[4-(methylsulfonyl)phenyl]-4-trifluoromethyl-1H-imidazole (D-142);
4-[2-(3-chloro-4-methylphenyl)-4-(trifluoromethyl)-1H-imidazol-1-yl]benzenesulfonamide (D-143);
2-(3-fluoro-5-methylphenyl)-1-[4-(methylsulfonyl)phenyl]-4-(trifluoromethyl)-1H-imidazole (D-144);
4-[2-(3-fluoro-5-methylphenyl)-4-(trifluoromethyl)-1H-imidazol-1-yl]benzenesulfonamide (D-145);
2-(3-methylphenyl)-1-[4-(methylsulfonyl)phenyl]-4-trifluoromethyl-1H-imidazole (D-146);
4-[2-(3-methylphenyl)-4-trifluoromethyl-1H-imidazol-1-yl]benzenesulfonamide (D-147);
1-[4-(methylsulfonyl)phenyl]-2-(3-chlorophenyl)-4-trifluoromethyl-1H-imidazole (D-148);
4-[2-(3-chlorophenyl)-4-trifluoromethyl-1H-imidazol1-yl]benzenesulfonamide (D-149);
4-[2-phenyl-4-trifluoromethyl-1H-imidazol-1-yl]benzenesulfonamide (D-150);
4-[2-(4-methoxy-3-chlorophenyl)-4-trifluoromethyl-1H-imidazol-1-yl]benzenesulfonamide (D-151);
1-allyl-4-(4-fluorophenyl)-3-[4-(methylsulfonyl)phenyl]-5-(trifluoromethyl)-1H-pyrazole (D-152);
4-[1-ethyl-4-(4-fluorophenyl)-5-(trifluoromethyl)-1H-pyrazol-3-yl]benzenesulfonamide (D-153);
N-phenyl-[4-(4-fluorophenyl)-3-[4-(methylsulfonyl)phenyl]-5-(trifluoromethyl)-1H-pyrazol-1-yl]acetamide (D-154);
ethyl [4-(4-fluorophenyl)-3-[4-(methylsulfonyl)phenyl]-5-(trifluoromethyl)-1H-pyrazol-1-yl]acetate (D-155);
4-(4-fluorophenyl)-3-[4-(methylsulfonyl)phenyl]-1-(2-phenylethyl)-1H-pyrazole (D-156);
4-(4-fluorophenyl)-3-[4-(methylsulfonyl)phenyl]-l-(2-phenylethyl)-5-(trifluoromethyl)pyrazole (D-157);
1-ethyl-4-(4-fluorophenyl)-3-[4-(methylsulfonyl)phenyl]-5-(trifluoromethyl)-1H-pyrazole (D-158);
5-(4-fluorophenyl)-4-(4-methylsulfonylphenyl)-2-trifluoromethyl-1H-imidazole (D-159);
4-[4-(methylsulfonyl)phenyl]-5-(2-thiophenyl)-2-(trifluoromethyl)-1H-imidazole (D-160);
5-(4-fluorophenyl)-2-methoxy-4-[4-(methylsulfonyl)phenyl)-6-(trifluoromethyl)pyridine (D-161);
2-ethoxy-5-(4-fluorophenyl)-4-[4-(methylsulfonyl)phenyl]-6-(trifluoromethyl)pyridine (D-162);
5-(4-fluorophenyl)-4-[4-(methylsulfonyl)phenyl]-2-(2-propynyloxy)-6-(trifluoromethyl)pyridine (D-163);
2-bromo-5-(4-fluorophenyl)-4-[4-(methylsulfonyl)phenyl]-6-(trifluoromethyl)pyridine (D-164);
4-[2-(3-chloro-4-methoxyphenyl)-4,5-difluorophenyl]benzenesulfonamide (D-165);
1-(4-fluorophenyl)-2-[4-(methylsulfonyl)phenyl]benzene (D-166);
5-difluoromethyl-4-(4-methylsulfonylphenyl)-3-phenylisoxazole (D-167);
4-[3-ethyl-5-phenylisoxazol-4-yl]benzenesulfonamide (D-168);
4-[5-difluoromethyl-3-phenylisoxazol-4-yl]benzenesulfonamide (D-169);
4-[5-hydroxymethyl-3-phenylisoxazol-4-yl]benzenesulfonamide (D-170);
4-[5-methyl-3-phenyl-isoxazol-4-yl]benzenesulfonamide (D-171);
1-[2-(4-fluorophenyl)cyclopenten-1-yl]-⁴-(methylsulfonyl)benzene (D-172);
1-[2-(4-fluoro-2-methylphenyl)cyclopenten-1-yl]-4-(methylsulfonyl)benzene (D-173);
1-[2-(4-chlorophenyl)cyclopenten-1-yl]-4-(methylsulfonyl)benzene (D-174);
1-[2-(2,4-dichlorophenyl)cyclopenten-1-yl]-4-(methylsulfonyl)benzene (D-175);
1-[2-(4-trifluoromethylphenyl)cyclopenten-1-yl]-4-(methylsulfonyl)benzene (D-176);
1-[2-(4-methylthiophenyl)cyclopenten-1-yl]-4-(methylsulfonyl)benzene (D-177);
1-[2-(4-fluorophenyl)-4,4-dimethylcyclopenten-1-yl]-4-(methylsulfonyl)benzene (D-178);
4-[2-(4-fluorophenyl)-4,4-dimethylcyclopenten-1-yl]benzenesulfonamide (D-179);
1-[2-(4-chlorophenyl)-4,4-dimethylcyclopenten-1-yl]-4-(methylsulfonyl)benzene (D-180);
4-[2-(4-chlorophenyl)-4,4-dimethylcyclopenten-1-yl]benzenesulfonamide (D-181);
4-[2-(4-fluorophenyl)cyclopenten-1-yl]benzenesulfonamide (D-182);
4-[2-(4-chlorophenyl)cyclopenten-1-yl]benzenesulfonamide (D-183);
1-[2-(4-methoxyphenyl)cyclopenten-1-yl]-4-(methylsulfonyl)benzene (D-184);
1-[2-(2,3-difluorophenyl)cyclopenten-1-yl]-4-(methylsulfonyl)benzene (D-185);
4-[2-(3-fluoro-4-methoxyphenyl)cyclopenten-1-y]benzenesulfonamide (D-186);
1-[2-(3-chloro-4-methoxyphenyl)cyclopenten-1-yl]-4-(methylsulfonyl)benzene (D-187);
4-[2-(3-chloro-4-fluorophenyl)cyclopenten-1-yl]benzenesulfonamide (D-188);
4-[2-(2-methylpyridin-5-yl)cyclopenten-1-yl]benzenesulfonamide (D-189); ethyl 2-[4-(4-fluorophenyl)-5-[4-(methylsulfonyl)phenyl]oxazol-2-yl]-2-benzyl-acetate (D-190);
2-[4-(4-fluorophenyl)-5-[4-(methylsulfonyl)phenyl]oxazol-2-yl]acetic acid (D-191);
2-(tert-butyl)-4-(4-fluorophenyl)-5-[4-(methylsulfonyl)phenyl]oxazole (D-192);
4-(4-fluorophenyl)-5-[4-(methylsulfonyl)phenyl]-2-phenyloxazole (D-193);
4-(4-fluorophenyl)-2-methyl-5-[4-(methylsulfonyl)phenyl]oxazole (D-194);
4-[5-(3-fluoro-4-methoxyphenyl)-2-trifluoromethyl-4-oxazolyl]benzenesulfonamide (D-195);
6-chloro-7-(1,1-dimethylethyl)-2-trifluoromethyl-2H-1-benzopyran-3-carboxylic acid (D-196);
6-chloro-8-methyl-2-trifluoromethyl-2h-1-benzopyran-3-carboxylic acid (D-197);
5,5-dimethyl-3-(3-fluorophenyl)-4-(4-methyl-sulphonyl-2(5H)-fluranone (D-198);
6-chloro-2-trifluoromethyl-2H-1-benzothiopyran-3-carboxylic acid (D-199);
4-[5-(4-chlorophenyl)-3-(trifluoromethyl)-1H-pyrazol-1-yl]benzenesulfonamide (D-200);
4-[5-(4-methylphenyl)-3-(trifluoromethyl)-1H-pyrazol-1-yl]benzenesulfonamide (D-201);
4-[5-(3-fluoro-4-methoxyphenyl)-3-(difluoromethyl)-1H-pyrazol-1-yl]benzenesulfonamide (D-202);
3-[1-[4-(methylsulfonyl)phenyl]-4-trifluoromethyl-1H-imidazol-2-yl]pyridine (D-203);
2-methyl-5-[1-[4-(methylsulfonyl)phenyl)-4-trifluoromethyl-1H-imidazol-2-yl]pyridine (D-204);
4-[2-(5-methylpyridin-3-yl)-4-(trifluoromethyl)-1H-imidazol-1-yl]benzenesulfonamide (D-205);
4-[5-methyl-3-phenylisoxazol-4-yl]benzenesulfonamide (D-206);
4-[5-hydroxymethyl-3-phenylisoxazol-4-yl]benzenesulfonamide (D-207);
[2-trifluoromethyl-5-(3,4-difluorophenyl)-4-oxazolyl]benzenesulfonamide (D-208);
4-[2-methyl-4-phenyl-5-oxazolyl]benzenesulfonamide (D-209);
4-[5-(3-fluoro-4-methoxyphenyl-2-trifluoromethyl)-4-oxazolyl]benzenesulfonamide (D-210);
[2-(2-Chloro-6-fluoro-phenylamino)-5-methyl-phenyl]-acetic acid, COX 189 (D-211);
N-(4-nitro-2-phenoxy-phenyl)methanesulfonamide, Nimesulide (D-212);
N-[6-(2,4-Difluoro-phenoxy)-1-oxo-indan-5-yl]-methanesulfonamide, Flosulide (D-213);
N-[6-(2,4-difluoro-phenylsulfonyl)-1-1-oxo-1H-inden-5-yl]-methanesulfonmaide, sodium salt, or L-745337 (D-214);
N-[5, (4-fluoro-phenylsulfanyl)-thiophen-2-yl]methanesulfonamide or RWJ-63556 (D-215);
(5Z)-2-amino-5-[[3,5-bis(1,1-dimethylethyl)-4-hydroxyphenyl]methylene]-4(5H)-thiazolone, Darbufelone (D-217);
N-[3-(formylamino)-4-oxo-6-phenoxy-4H-1-benzopyran-7-yl]-methanesulfonamide, T-614 (D-224);
(6aR,10aR)-3-(1,1-dimethylheptyl)-6a,7,10,10a-tetrahydro-1-hydroxy-6,6-dimethyl-6H-dibenzo[b,d]pyran-9-carboxylic acid, CT3 (D-227);
4-[[3,5-bis(1,1-dimethylethyl)-4-hydroxyphenyl]methylene]dihydro-2-methyl-2H-1,2-oxazin-3(4H)-one, BF-389 (D-229);
6-dioxo-9H-purin-8-yl-cinnamic acid (D-231);
or a pharmaceutically acceptable salt or derivative or prodrug thereof.

18. The kit of claim 16 wherein the cyclooxygenase-2 selective inhibitor is D-1 to D-5, D-6 to D-10, D-11 to D-15, D-16 to D-20, D-21 to D-25, D-26 to D-30, D-31 to D-35, D-36 to D-40, D-41 to D-45, D-46 to D-50, D-51 to D-55, D-56 to D-60, D-61 to D-65, D-66 to D-70, D-71 to D-75, D-76 to D-80, D-81 to D-85, D-86 to D-90, D-91 to D-95, D-96 to D-100, D-101 to D-105, D-106 to D-110, D-111 to D-115, D-116 to D-120, D-121 to D-125, D-126 to D-130, D-131 to D-135, D-136 to D-140, D-141 to D-145, D-146 to D-150, D-151 to D-155, D-156 to D-160, D-161 to D-165, D-166 to D-170, D-171 to D-175, D-176 to D-180, D-181 to D-185, D-186 to D-190, D-191 to D-195, D-196 to D-200, D-201 to D-205, D-206 to D-210, D-211 to D-215, D-217, D-224, D-227, D-229, D-231, or a pharmaceutically acceptable salt or derivative or prodrug thereof.

19. The kit of claim 16 further comprising an amount of an HMG-CoA reductase inhibitor wherein the amount of the apical sodium co-dependent bile acid transport inhibitor, the amount of the cyclooxygenase-2 selective inhibitor and the amount of the HMG-CoA reductase inhibitor together constitute a hypercholesterolemia-related condition effective amount or an inflammation-related condition effective amount of the apical sodium co-dependent bile acid transport inhibitor, the cyclooxygenase-2 selective inhibitor and the HMG-CoA reductase inhibitor.

20. The kit of claim 19 wherein the HMG-CoA reductase inhibitor is selected from the group consisting of fluvastatin, lovastatin, pravastatin, simvastatin, atorvastatin, cerivastatin, bervastatin, rosuvastatin, and itavastatin, or a pharmaceutically acceptable salt or ester or lactone thereof.
